SEQUENCE LISTING (22 sequences)

SEQ ID NO: 1 - RNA, 20 nt, synthetic construct
  source 1..20: mol_type = other RNA
ggggctgtgggaacggtcct

SEQ ID NO: 2 - RNA, 20 nt, synthetic construct
  source 1..20: mol_type = other RNA
ggcccaatgggctgtgggaa

SEQ ID NO: 3 - RNA, 20 nt, synthetic construct
  source 1..20: mol_type = other RNA
gcccacgcagagtgcgctca

SEQ ID NO: 4 - RNA, 20 nt, synthetic construct
  source 1..20: mol_type = other RNA
gcgggcgcccgtccaatcac

SEQ ID NO: 5 - RNA, 20 nt, synthetic construct
  source 1..20: mol_type = other RNA
gccagggagcaggaaatcgg

SEQ ID NO: 6 - RNA, 20 nt, synthetic construct
  source 1..20: mol_type = other RNA
gggatcctggacaggcaccc

SEQ ID NO: 7 - RNA, 20 nt, synthetic construct
  source 1..20: mol_type = other RNA
ggactcaccggacggacgtg

SEQ ID NO: 8 - RNA, 20 nt, synthetic construct
  source 1..20: mol_type = other RNA
gggcggcacccgggactcac

SEQ ID NO: 9 - DNA, 20 bp, synthetic construct
  source 1..20: mol_type = other DNA
agagctacgagctgcctgac

SEQ ID NO: 10 - DNA, 20 bp, synthetic construct
  source 1..20: mol_type = other DNA
agcactgtgttggcgtacag

SEQ ID NO: 11 - DNA, 19 bp, synthetic construct
  source 1..19: mol_type = other DNA
tgtccgtcagaacccatgc

SEQ ID NO: 12 - DNA, 21 bp, synthetic construct
  source 1..21: mol_type = other DNA
aaagtcgaagttccatcgctc

SEQ ID NO: 13 - DNA, 23 bp, synthetic construct
  source 1..23: mol_type = other DNA
aatcagagtacatgcgactgaga

SEQ ID NO: 14 - DNA, 21 bp, synthetic construct
  source 1..21: mol_type = other DNA
gctgtatccttcgctgtttcc

SEQ ID NO: 15 - DNA, 22 bp, synthetic construct
  source 1..22: mol_type = other DNA
acgcctggttactatcaaaagg

SEQ ID NO: 16 - DNA, 23 bp, synthetic construct
  source 1..23: mol_type = other DNA
cagacccatttacttgtgttgga

SEQ ID NO: 17 - DNA, 20 bp, synthetic construct
  source 1..20: mol_type = other DNA
cagcagcttgacacacggta

SEQ ID NO: 18 - DNA, 21 bp, synthetic construct
  source 1..21: mol_type = other DNA
aaacaccaaagtggcatgtga

SEQ ID NO: 19 - DNA, 21 bp, synthetic construct
  source 1..21: mol_type = other DNA
cttctgcaattccgacctcgt

SEQ ID NO: 20 - DNA, 21 bp, synthetic construct
  source 1..21: mol_type = other DNA
acgctggtataaggtggtctg

SEQ ID NO: 21 - DNA, 19 bp, synthetic construct
  source 1..19: mol_type = other DNA
ggcagtccacagcaagaac

SEQ ID NO: 22 - DNA, 23 bp, synthetic construct
  source 1..23: mol_type = other DNA
ttggtatccgagaaagtcagaag

(19) United States
(12) Patent Application Publication
(10) Pub. No.: US 2026/0092258 A1
(43) Pub. Date: Apr. 2, 2026

(54) Method to Rejuvenate Human Cells Through Transcriptional Reprogramming

(71) Applicant: The Regents of the University of California, Oakland, CA (US)

(72) Inventors: Janine Sengstack, San Francisco, CA (US); Changhui Deng, San Francisco, CA (US); Jiashun Zheng, San Francisco, CA (US); Hao Li, San Francisco, CA (US)

(21) Appl. No.: 19/113,667

(22) PCT Filed: Sep. 25, 2023
(86) PCT No.: PCT/US2023/075057

§ 371 Date: Mar. 20, 2025

Related U.S. Application Data
(60) Provisional application No. 63/377,250, filed on Sep. 27, 2022.

(51) Int. Cl. C12N 5/077; C12N 9/22; C12N 15/11; C12N 15/113

(52) U.S. Cl. CPC C12N 5/0656; C12N 9/22; C12N 15/111; C12N 15/113

(57) ABSTRACT

Methods and compositions for rejuvenating cells are provided. In certain aspects, the method includes increasing, in the cells, the activity of one or more of the transcription factors (TFs) presented herein. In certain aspects, the method includes decreasing, in the cells, the activity of one or more of the TFs presented herein. In certain aspects, the method includes increasing activity of one or more and decreasing the activity of one or more of TFs presented herein

### CROSS-REFERENCE TO RELATED APPLICATIONS

This application claims priority to U.S. Provisional Application No. 63/377,250, filed on Sep. 27, 2022, which application is incorporated by reference in its entirety.

### STATEMENT AS TO FEDERALLY SPONSORED RESEARCH

This invention was made with government support under grant R21 AG064357 awarded by The National Institutes of Health. The government has certain rights in the invention.

### REFERENCE TO AN ELECTRONIC SEQUENCE LISTING

The contents of the electronic sequence listing (UCSF-687WO_SEQ_LIST.xml; Size: 20,475 bytes; and Date of Creation: Sep. 18, 2023) are herein incorporated by reference in its entirety.

### INTRODUCTION

Cellular rejuvenation is an approach to counteract aging. If old cells can be made to behave like young cells again, rejuvenation of tissues, organs, or even the whole body, and alleviation of aging associated diseases is possible. So far, the Yamanaka factors are the only known genes capable of cellular rejuvenation. Unfortunately, there is considerable cancer risk to overexpressing these genes due to their ability to transform differentiated cells to stem cells, which may then grow into tumors. As such there is a need for improved methods for cellular rejuvenation.

### SUMMARY

Provided herein is a method for rejuvenating a cell, the method comprising: (a) increasing the activity of at least one transcription factor selected from: (i) E2F Transcription Factor 3 (E2F3); and (ii) Enhancer Of Zeste 2 Polycomb Repressive Complex 2 Subunit (EZH2): or (b) decreasing the activity of at least one transcription factor selected from: (i) Signal Transducer And Activator Of Transcription 3 (STAT3); and (ii) Zinc Finger Protein X-Linked (ZFX).

Also provided herein is a method for rejuvenating a cell the method comprising: (a) increasing the activity of at least one transcription factor selected from: E2F Transcription Factor 3 (E2F3); and Enhancer Of Zeste 2 Polycomb Repressive Complex 2 Subunit (EZH2); and decreasing the activity of at least one transcription factor selected from: Signal Transducer And Activator Of Transcription 3 (STAT3); and Zinc Finger Protein X-Linked (ZFX).

In some embodiments, the level of the activity of the at least one transcription factor is increased or decreased, thereby rejuvenating the cell.

In some embodiments, the method comprises increasing the activities of E2F3 and EZH2. In some embodiments, the method comprises decreasing the activities of STAT3 and ZFX. In some embodiments, the method comprises increasing the activity of E2F3 and decreasing the activity of STAT3. In some embodiments, the method comprises increasing the activity of E2F3 and decreasing the activity of ZFX. In some embodiments, the method comprises increasing the activity of EZH2 and decreasing the activity of STAT3. In some embodiments, the method comprises increasing the activities of EZH2 and decreasing the activity of ZFX. In some embodiments, the method comprises increasing the activities of E2F3 and EZH2 and decreasing the activity of STAT3. In some embodiments, the method comprises increasing the activities of E2F3 and EZH2 and decreasing the activity of ZFX. In some embodiments, the method comprises increasing the activity of EZH2 and decreasing the activities of STAT3 and ZFX. In some embodiments, the method comprises increasing the activities of E2F3 and EZH2 and decreasing the activities of STAT3 and ZFX.

In some embodiments, the cell is a human cell.

In some embodiments, the cell is an adult human cell. In some embodiments, the cell is a primary human cell. In some embodiments, the cell is a primary adult human cell. In some embodiments, the cell is a differentiated cell. In some embodiments, the cell is an epithelial cell. In some embodiments, the cell is a skin cell, a lung cell, a liver cell, a muscle cell, a pancreatic cell, an immune cell, a bone cell, or a brain cell. In some embodiments, the cell is a fibroblast. In some embodiments, the cell is a skin fibroblast. In some embodiments, the cell is a liver fibroblast.

In some embodiments, the method results in an increase in cell division from the cell as compared to a corresponding untreated cell. In some embodiments, the method results in at least 1%, 2%, 3%, 4%, or 5% increase in cell division. In some embodiments, the method results in a cell that is capable of increasing cell division as compared to a corresponding untreated cell. In some embodiments, the cell is capable of increasing cell division by at least 1%, 2%, 3%, 4%, or 5%. In some embodiments, the increase in cell division is determined by an increase of the number of KI67 positive cells in a cell population.

In some embodiments, the method results in a decrease in expression of at least one senescence related gene in the cell as compared to a corresponding untreated cell. In some embodiments, the at least one senescence related gene is selected from the group consisting of p21, TIMP1, and TIMP2. In some embodiments, the method results in at least 1%, 2%, 3%, 4%, or 5% decrease in expression of the at least one senescence related gene. In some embodiments, the decrease in expression of the at least one senescence related gene is determined by an increase of the number of beta-galactosidase-positive cells.

In some embodiments, the method results in an increase in proteasome activity in the cell as compared to a corresponding untreated cell. In some embodiments, the method results in at least 1%, 2%, 3%, 4%, or 5% increase in the proteasome activity. In some embodiments, the method results in a cell that is capable of increasing proteasome activity as compared to a corresponding untreated cell. In some embodiments, the cell is capable of increasing proteasome activity by at least 1%, 2%, 3%, 4%, or 5%. In some embodiments, the increased proteasome activity is determined by an increase of proteasome-mediated cleavage of a substrate. In some embodiments, the increased proteasome activity is measured by a fluorescence-based cleavage assay.

In some embodiments, the method results in a decrease in senescence-associated lysosomes in the cell as compared to a corresponding untreated cell. In some embodiments, the method results in at least 1%, 2%, 3%, 4%, or 5% decrease in the senescence-associated lysosomes. In some embodiments, the decrease in the senescence-associated lysosomes is determined by a decrease of the number of lysosome puncta per cell area. In some embodiments, the number of lysosome puncta per cell area is measured by Lysotracker™ staining.

In some embodiments, the method results in an increase in mitochondrial and metabolism gene expression in the cell as compared to a corresponding untreated human adult cell. Exemplary mitochondrial and metabolism genes which may have increased expression include one or more of MT-ATP6, MT-ATP8, MT-CYB, MT-CO1, MT-CO2, MT-CO3, MT-ND4L, MT-ND1, MT-ND2, MT-ND3, MT-ND4, MT-ND5, MT-ND6, ACLY, ACO1, ACO2, CS, DLAT, DLD, DLST, FH, IDH1, IDH2, IDH3A, IDH3B, IDH3G, MDH1, MDH2, OGDH, OGDHL, PC, PCK1, PCK2, PDHA1, PDHA2, PDHB, SDHA, SDHB, SDHC, SDHD, SUCLA2, SUCLG1, and SUCLG2.

In some embodiments, the method results in an increase in mitochondrial membrane potential in the cell as compared to a corresponding untreated cell. In some embodiments, the increase in mitochondrial membrane potential is measured by TMRE (tetramethylrhodamine, ethyl ester) membrane potential staining.

In some embodiments, the method further comprises: increasing the activity of at least one transcription factor selected from: Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); or decreasing the activity of at least one transcription factor selected from: Early growth response protein 1 (EGR1); Activating Transcription Factor 2 (ATF2); Vascular Endothelial Zinc Finger 1 (VEZF1); MYC Associated Zinc Finger Protein (MAZ); SRY-Box Transcription Factor 2 (SOX2); Paired Box 8 (PAX8); Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4).

In some embodiments, the method comprises increasing the activity of E2F3 in the cell. In some embodiments, increasing the activity of E2F3 comprises increasing the mRNA level of E2F3 in the cell. In some embodiments, the mRNA level of E2F3 is increased by at least 1%, 2%, 3%, 4%, 5%, or 10% as compared to a corresponding untreated cell. In some embodiments, increasing the activity of E2F3 comprises increasing the protein level of E2F3 in the cell. In some embodiments, the protein E2F3 is increased by at least 1%, 2%, 3%, 4%, 5%, or 10% as compared to a corresponding untreated cell.

In some embodiments, the method comprises increasing the activity of EZH2 in the cell.

In some embodiments, increasing the activity of EZH2 comprises increasing the mRNA level of EZH2 in the cell. In some embodiments, the mRNA level of EZH2 is increased by at least 1%, 2%, 3%, 4%, 5%, or 10% as compared to a corresponding untreated cell. In some embodiments, increasing the activity of EZH2 comprises increasing the protein level of EZH2 in the cell. In some embodiments, the protein level of EZH2 is increased by at least 1%, 2%, 3%, 4%, 5%, or 10% as compared to a corresponding untreated cell.

In some embodiments, the method comprises increasing the activity of DLX6 in the cell.

In some embodiments, increasing the activity of DLX6 comprises increasing the mRNA level of DLX6 in the cell. In some embodiments, the mRNA level of DLX6 is increased by at least 1%, 2%, 3%, 4%, 5%, or 10% as compared to a corresponding untreated cell. In some embodiments, increasing the activity of DLX6 comprises increasing the protein level of DLX6 in the cell. In some embodiments, the protein level of DLX6 is increased by at least 1%, 2%, 3%, 4%, 5%, or 10% as compared to a corresponding untreated cell.

In some embodiments, the method comprises increasing the activity of FOXM1 in the cell. In some embodiments, increasing the activity of FOXM1 comprises increasing the mRNA level of FOXM1 in the cell. In some embodiments, the mRNA level of FOXM1 is increased by at least 1%, 2%, 3%, 4%, 5%, or 10% as compared to a corresponding untreated cell. In some embodiments, increasing the activity of FOXM1 comprises increasing the protein level of FOXM1 in the cell. In some embodiments, the protein FOXM1 is increased by at least 1%, 2%, 3%, 4%, 5%, or 10% as compared to a corresponding untreated cell.

In some embodiments, increasing the activity of the at least one transcription factor comprises introducing into the cell one or more agents comprising a sequence encoding the at least one transcription factor, thereby increasing the activity of the at least one transcription factor. In some embodiments, increasing the activity of the at least one transcription factor comprises introducing into the cell one or more agents that increase expression of the at least one transcription factor, thereby increasing the activity of the at least one transcription factor. In some embodiments, increasing the activity of the at least one transcription factor comprises introducing into the cell one or more agents that increase transcription or translation of the at least one transcription factor, thereby increasing the activity of the at least one transcription factor.

In some embodiments, the one or more agents are one or more polynucleic acids. In some embodiments, the one or more agents are encoded by one or more polynucleic acids. In some embodiments, the one or more polynucleic acids comprise a viral vector. In some embodiments, the one or more polynucleic acids comprise a non-viral vector. In some embodiments, the one or more polynucleic acids are encapsulated in a lipid nanoparticle (LNP).

In some embodiments, increasing the activity of the at least one transcription factor comprises introducing into the cell one or more polynucleic acids comprising a sequence encoding the at least one transcription factor, thereby increasing transcription from the at least one transcription factor. In some embodiments, increasing the activity of E2F3 comprises introducing into the cell one or more polynucleic acids comprising a sequence encoding E2F3. In some embodiments, increasing the activity of EZH2 comprises introducing into the cell one or more polynucleic acids comprising a sequence encoding EZH2. In some embodiments, increasing the activity of DLX6 comprises introducing into the cell one or more polynucleic acids comprising a sequence encoding DLX6. In some embodiments, increasing the activity of FOXM1 comprises introducing into the cell one or more polynucleic acids comprising a sequence encoding FOXM1. In some embodiments, the method comprises introducing into the cell one or more polynucleic acids comprising (i) a sequence encoding E2F3, and (ii) a sequence encoding EZH2. In some embodiments, the method comprises introducing into the cell one or more polynucleic acids comprising a sequence encoding DLX6. In some embodiments, the method comprises introducing into the cell one or more polynucleic acids comprising a sequence encoding FOXM1.

In some embodiments, the one or more polynucleic acids comprise a viral vector. In some embodiments, the one or more polynucleic acids comprise a non-viral vector. In some embodiments, the one or more polynucleic acids are encapsulated in a lipid nanoparticle (LNP).

In some embodiments, the method comprises decreasing the activity of STAT3 in the cell. In some embodiments, decreasing the activity of STAT3 comprises decreasing the mRNA level of STAT3 in the cell. In some embodiments, the mRNA level of STAT3 is decreased by at least 1%, 2%, 3%, 4%, 5%, or 10% as compared to a corresponding untreated cell. In some embodiments, decreasing the activity of STAT3 comprises decreasing the protein level of STAT3 in the cell. In some embodiments, the protein level of STAT3 is decreased by at least 1%, 2%, 3%, 4%, 5%, or 10% as compared to a corresponding untreated cell.

In some embodiments, the method comprises decreasing the activity of ZFX in the cell.

In some embodiments, decreasing the activity of ZFX comprises decreasing the mRNA level of ZFX in the cell. In some embodiments, the mRNA level of ZFX is decreased by at least 1%, 2%, 3%, 4%, 5%, or 10% as compared to a corresponding untreated cell. In some embodiments, decreasing the activity of ZFX comprises decreasing the protein level of ZFX in the cell. In some embodiments, the protein level of ZFX is decreased by at least 1%, 2%, 3%, 4%, 5%, or 10% as compared to a corresponding untreated cell.

In some embodiments, the method comprises decreasing the activity of EGR1 in the cell. In some embodiments, decreasing the activity of EGR1 comprises decreasing the mRNA level of EGR1 in the cell. In some embodiments, the mRNA level of EGR1 is decreased by at least 1%, 2%, 3%, 4%, 5%, or 10% as compared to a corresponding untreated cell. In some embodiments, decreasing the activity of EGR1 comprises decreasing the protein level of EGR1 in the cell. In some embodiments, the protein level of EGR1 is decreased by at least 1%, 2%, 3%, 4%, 5%, or 10% as compared to a corresponding untreated cell.

In some embodiments, the method comprises decreasing the activity of MAZ in the cell. In some embodiments, decreasing the activity of MAZ comprises decreasing the mRNA level of MAZ in the cell. In some embodiments, the mRNA level of MAZ is decreased by at least 1%, 2%, 3%, 4%, 5%, or 10% as compared to a corresponding untreated cell. In some embodiments, decreasing the activity of MAZ comprises decreasing the protein level of MAZ in the cell. In some embodiments, the protein level of MAZ is decreased by at least 1%, 2%, 3%, 4%, 5%, or 10% as compared to a corresponding untreated cell.

In some embodiments, the method comprises decreasing the activity of SOX2 in the cell. In some embodiments, decreasing the activity of SOX2 comprises decreasing the mRNA level of SOX2 in the cell. In some embodiments, the mRNA level of SOX2 is decreased by at least 1%, 2%, 3%, 4%, 5%, or 10% as compared to a corresponding untreated cell. In some embodiments, decreasing the activity of SOX2 comprises decreasing the protein level of SOX2 in the cell. In some embodiments, the protein level of SOX2 is decreased by at least 1%, 2%, 3%, 4%, 5%, or 10% as compared to a corresponding untreated cell.

In some embodiments, the method comprises decreasing the activity of ATF4 in the cell. In some embodiments, decreasing the activity of ATF4 comprises decreasing the mRNA level of ATF4 in the cell. In some embodiments, the mRNA level of ATF4 is decreased by at least 1%, 2%, 3%, 4%, 5%, or 10% as compared to a corresponding untreated cell. In some embodiments, decreasing the activity of ATF4 comprises decreasing the protein level of ATF4 in the cell. In some embodiments, the protein level of ATF4 is decreased by at least 1%, 2%, 3%, 4%, 5%, or 10% as compared to a corresponding untreated cell.

In some embodiments, decreasing the activity of the at least one transcription factor comprises introducing into the cell one or more agents for decreasing transcription from the at least one transcription factor.

In some embodiments, decreasing the activity of the at least one transcription factor comprises introducing into the cell one or more agents that decrease expression of the at least one transcription factor, thereby decreasing the activity of the at least one transcription factor. In some embodiments, decreasing the activity of the at least one transcription factor comprises introducing into the cell one or more agents that decrease transcription or translation of the at least one transcription factor, thereby decreasing activity of the at least one transcription factor.

In some embodiments, the one or more agents comprise a CRISPR system, a repression plasmid, an antisense oligonucleotide, a miRNA or an siRNA.

In some embodiments, the one or more agents are one or more polynucleic acids. In some embodiments, the one or more polynucleic acids encode the CRISPR system, the antisense oligonucleotide, or the siRNA. In some embodiments, the one or more polynucleic acids comprise a viral vector. In some embodiments, the one or more polynucleic acids comprise a non-viral vector. In some embodiments, the one or more polynucleic acids are encapsulated in a lipid nanoparticle (LNP).

In some embodiments, decreasing the activity of STAT3 comprises introducing into the cell an inhibitor of STAT3. In some embodiments, decreasing the activity of ZFX comprises introducing into the cell an inhibitor of ZFX.

In some embodiments, the cell is ex vivo. In some embodiments, the cell is in situ. In some embodiments, the cell is in vivo. In some embodiments, the cell is from a human subject. In some embodiments, the human subject that does not have psoriasis. In some embodiments, the human subject does not have psoriasis and the cells are in situ. In some embodiments, the human subject does not have psoriasis and the cells are in vivo.

In some embodiments, the cell is from a human subject having an age of at least 30 years, at least 35 years, at least 40 years, at least 45 years, at least 50 years, at least 55 years, at least 60 years, at least 65 years, at least 70 years, at least 75 years, at least 80 years, at least 85 years, or at least 90 years.

In some embodiments, the cell, after treatment, does not comprise substantial changes of TERT-related gene expression or telomere length. Exemplary TERT-related genes include EREG, EEF1A2, ALDH1A1, and EPB41L3.

In some embodiments, the method does not comprise increasing the activity of any one of OCT4, SOX2, KLF4, and MYC. In some embodiments, the method does not comprise increasing the activity of any of OCT4, SOX2, KLF4, MYC, or any combination thereof. In some embodiments, the method does not comprise increasing the activity of OCT4, SOX2, KLF4, and MYC.

Also provided herein is a composition for rejuvenating cells, the composition comprising: at least one activator of a transcription factor, wherein the transcription factor is selected from the group consisting of: E2F Transcription Factor 3 (E2F3); Enhancer Of Zeste 2 Polycomb Repressive Complex 2 Subunit (EZH2); Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); and Ventral Anterior Homeobox 1 (VAX1); and at least one inhibitor of a transcription factor, wherein the transcription factor is selected from the group consisting of: Signal Transducer And Activator Of Transcription 3 (STAT3); Zinc Finger Protein X-Linked (ZFX); Early growth response protein 1 (EGR1); Activating Transcription Factor 2 (ATF2); Vascular Endothelial Zinc Finger 1 (VEZF1); MYC Associated Zinc Finger Protein (MAZ); SRY-Box Transcription Factor 2 (SOX2); Paired Box 8 (PAX8); Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4).

In some embodiments, the composition comprises: (a) at least one activator of a transcription factor of EZH2, E2F3, FOXM1, or DLX6; and (b) at least one inhibitor of a transcription factor of STAT3, ZFX, EGR1, MAZ, SOX2, or ATF4.

In some embodiments, the composition does not comprise an activator of a transcription factor of OCT4, SOX2, KLF4, or MYC.

In some embodiments, the composition comprises (a) an activator of a transcription factor of EZH2 or E2F3; and (b) an inhibitor of a transcription factor of STAT3 or ZFX.

In some embodiments, the composition comprises an activator of E2F3 and an activator of EZH2. In some embodiments, the composition comprises an inhibitor of STAT3 and an inhibitor of ZFX. In some embodiments, the composition comprises an activator of E2F3 and an inhibitor of STAT3. In some embodiments, the composition comprises an activator of E2F3 and an inhibitor of ZFX. In some embodiments, the composition comprises an activator of EZH2 and an inhibitor of STAT3. In some embodiments, the composition comprises an activator of EZH2 and an inhibitor of ZFX. In some embodiments, the composition comprises an activator of E2F3, an activator of EZH2 and an inhibitor of STAT3. In some embodiments, the composition comprises an activator of E2F3, an activator of EZH2 and an inhibitor of ZFX. In some embodiments, the composition comprises an activator of EZH2, an inhibitor of STAT3, and an inhibitor of ZFX. In some embodiments, the composition comprises an activator of E2F3, an activator of EZH2, an inhibitor of STAT3 and an inhibitor ZFX.

In some embodiments, the activator comprises a polynucleic acid comprising a sequence encoding the transcription factor or an expression plasmid comprising the sequence encoding the transcription factor.

In some embodiments, the inhibitor comprises a CRISPR system, a repression plasmid, an antisense oligonucleotide, an miRNA or siRNA targeting the transcription factor or a sequence encoding the transcription factor: or a polynucleic acid encoding the CRISPR system, the antisense oligonucleotide, the miRNA or the siRNA.

Also provided herein is a primary cell, comprising: at least one activator of a transcription factor, wherein the transcription factor is selected from the group consisting of: E2F Transcription Factor 3 (E2F3); Enhancer Of Zeste 2 Polycomb Repressive Complex 2 Subunit (EZH2); Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); and Ventral Anterior Homeobox 1 (VAX1); or at least one inhibitor of a transcription factor, wherein the transcription factor is selected from the group consisting of: Signal Transducer And Activator Of Transcription 3 (STAT3); Zinc Finger Protein X-Linked (ZFX); Early growth response protein 1 (EGR1); Activating Transcription Factor 2 (ATF2); Vascular Endothelial Zinc Finger 1 (VEZF1); MYC Associated Zinc Finger Protein (MAZ); SRY-Box Transcription Factor 2 (SOX2); Paired Box 8 (PAX8); Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4).

In some embodiments, the primary cell is a primary human cell. In some embodiments, the primary cell is a primary adult human cell.

In some embodiments, the primary cell comprises: (a) at least one activator of a transcription factor of EZH2, E2F3, FOXM1 or DLX6; or (b) at least one inhibitor of a transcription factor of STAT3, ZFX, EGR1, MAZ, SOX2, or ATF4. In some embodiments, the primary cell does not comprise an activator of a transcription factor of OCT4, SOX2, KLF4, or MYC. In some embodiments, the primary cell comprises (a) an activator of a transcription factor of EZH2 or E2F3; or (b) an inhibitor of a transcription factor of STAT3 or ZFX. In some embodiments, the primary cell comprises an activator of E2F3 and an activator of EZH2. In some embodiments, the primary cell comprises an inhibitor of STAT3 and an inhibitor of ZFX. In some embodiments, the primary cell comprises an activator of E2F3 and an inhibitor of STAT3. In some embodiments, the primary cell comprises an activator of E2F3 and an inhibitor of ZFX. In some embodiments, the primary cell comprises an activator of EZH2 and an inhibitor of STAT3. In some embodiments, the primary cell comprises an activator of EZH2 and an inhibitor of ZFX. In some embodiments, the primary cell comprises an activator of E2F3, an activator of EZH2 and an inhibitor of STAT3. In some embodiments, the primary cell comprises an activator of E2F3, an activator of EZH2 and an inhibitor of ZFX. In some embodiments, the primary cell comprises an activator of EZH2, an inhibitor of STAT3, and an inhibitor of ZFX. In some embodiments, the primary cell comprises an activator of E2F3, an activator of EZH2, an inhibitor of STAT3 and an inhibitor ZFX.

In some embodiments, the activator comprises a polynucleic acid comprising a sequence encoding the transcription factor or an expression plasmid comprising the sequence encoding the transcription factor.

In some embodiments, the inhibitor comprises a CRISPR system, a repression plasmid, an antisense oligonucleotide, or siRNA targeting the transcription factor; or a polynucleic acid encoding the CRISPR system, the antisense oligonucleotide, miRNA or the siRNA.

Also provided herein is a pharmaceutical composition comprising a composition that comprises: at least one activator of a transcription factor, wherein the transcription factor is selected from the group consisting of: E2F Transcription Factor 3 (E2F3); Enhancer Of Zeste 2 Polycomb Repressive Complex 2 Subunit (EZH2): Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); and Ventral Anterior Homeobox 1 (VAX1); or at least one inhibitor of a transcription factor, wherein the transcription factor is selected from the group consisting of: Signal Transducer And Activator Of Transcription 3 (STAT3); Zinc Finger Protein X-Linked (ZFX); Early growth response protein 1 (EGR1); Activating Transcription Factor 2 (ATF2); Vascular Endothelial Zinc Finger 1 (VEZF1); MYC Associated Zinc Finger Protein (MAZ); SRY-Box Transcription Factor 2 (SOX2); Paired Box 8 (PAX8); Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4); and a pharmaceutically acceptable carrier, excipient, or diluent.

In some embodiments, the composition comprises: (a) at least one activator of a transcription factor of EZH2, E2F3, FOXM1, or DLX6; or (b) at least inhibitor of a transcription factor of STAT3, ZFX, EGR1, MAZ, SOX2, or ATF4. In some embodiments, the composition does not comprise an activator of a transcription factor of OCT4, SOX2, KLF4, or MYC. In some embodiments, the composition comprises (a) an activator of a transcription factor of EZH2 or E2F3; or (b) an inhibitor of a transcription factor of STAT3 or ZFX. In some embodiments, the composition comprises an activator of E2F3 and an activator of EZH2. In some embodiments, the composition comprises an inhibitor of STAT3 and an inhibitor of ZFX. In some embodiments, the composition comprises an activator of E2F3 and an inhibitor of STAT3. In some embodiments, the composition comprises an activator of E2F3 and an inhibitor of ZFX. In some embodiments, the composition comprises an activator of EZH2 and an inhibitor of STAT3. In some embodiments, the composition comprises an activator of EZH2 and an inhibitor of ZFX. In some embodiments, the composition comprises an activator of E2F3, an activator of EZH2 and an inhibitor of STAT3. In some embodiments, the composition comprises an activator of E2F3, an activator of EZH2 and an inhibitor of ZFX. In some embodiments, the composition comprises an activator of EZH2, an inhibitor of STAT3, and an inhibitor of ZFX. In some embodiments, the composition comprises an activator of E2F3, an activator of EZH2, an inhibitor of STAT3 and an inhibitor ZFX.

In some embodiments, the activator comprises a polynucleic acid comprising a sequence encoding the transcription factor or an expression plasmid comprising the sequence encoding the transcription factor.

In some embodiments, the inhibitor comprises a CRISPR system, a repression plasmid, an antisense oligonucleotide, or siRNA targeting the transcription factor; or a polynucleic acid encoding the CRISPR system, the antisense oligonucleotide, miRNA or the siRNA.

Also provided herein is a LNP composition comprising a composition provided herein.

Also provided herein is a cell comprising a composition provided herein.

Also provided herein is a cell produced by a method provided herein.

Also provided herein is a polynucleotide comprising a composition provided herein.

Also provided herein is a pharmaceutical composition comprising (i) a composition provided herein, a primary cell provided herein, a cell provided herein, a polynucleotide provided herein, and (ii) a pharmaceutically acceptable carrier, excipient, or diluent.

Also provided herein is a method of treating or preventing a disease, condition or disorder in a subject in need thereof, the method comprising administration of any one pharmaceutical composition provided herein to the subject in need thereof.

In some embodiments, the disease, condition or disorder is an age-related disorder.

Also provided herein is a kit, comprising: at least one activator of a transcription factor, wherein the transcription factor is selected from the group consisting of: E2F Transcription Factor 3 (E2F3): Enhancer Of Zeste 2 Polycomb Repressive Complex 2 Subunit (EZH2); Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); and Ventral Anterior Homeobox 1 (VAX1); and at least one inhibitor of a transcription factor, wherein the transcription factor is selected from the group consisting of: Signal Transducer And Activator Of Transcription 3 (STAT3); Zinc Finger Protein X-Linked (ZFX); Early growth response protein 1 (EGR1); Activating Transcription Factor 2 (ATF2); Vascular Endothelial Zinc Finger 1 (VEZF1); MYC Associated Zinc Finger Protein (MAZ): SRY-Box Transcription Factor 2 (SOX2); Paired Box 8 (PAX8): Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4).

In some embodiments, the kit comprises: (a) at least one activator of a transcription factor of EZH2, E2F3, FOXM1, or DLX6; and (b) at least one inhibitor of a transcription factor of STAT3, ZFX, EGR1, MAZ, SOX2, or ATF4.

In some embodiments, the kit does not comprise an activator of a transcription factor of OCT4, SOX2, KLF4, or MYC. In some embodiments, the kit comprises (a) one or both activator of a transcription factor of EZH2 or E2F3; and (b) one or both inhibitor of a transcription factor of STAT3 or ZFX. In some embodiments, the kit comprises an activator of E2F3 and an activator of EZH2. In some embodiments, the kit comprises an inhibitor of STAT3 and an inhibitor of ZFX. In some embodiments, the kit comprises an activator of E2F3 and an inhibitor of STAT3. In some embodiments, the kit comprises an activator of E2F3 and an inhibitor of ZFX. In some embodiments, the kit comprises an activator of EZH2 and an inhibitor of STAT3. In some embodiments, the kit comprises an activator of EZH2 and an inhibitor of ZFX. In some embodiments, the kit comprises an activator of E2F3, an activator of EZH2 and an inhibitor of STAT3. In some embodiments, the kit comprises an activator of E2F3, an activator of EZH2 and an inhibitor of ZFX. In some embodiments, the kit comprises an activator of EZH2, an inhibitor of STAT3, and an inhibitor of ZFX. In some embodiments, the kit comprises an activator of E2F3, an activator of EZH2, an inhibitor of STAT3 and an inhibitor ZFX.

In some embodiments, the activator comprises a polynucleic acid comprising a sequence encoding the transcription factor or an expression plasmid comprising the sequence encoding the transcription factor.

In some embodiments, the inhibitor comprises a CRISPR system, a repression plasmid, an antisense oligonucleotide, miRNA or siRNA; or a polynucleic acid comprising a sequence encoding the CRISPR system, the antisense oligonucleotide, the miRNA or the siRNA.

Also provided herein is a method for rejuvenating a cell, the method comprising increasing activity of one or more transcription factors in the cell, wherein the one or more transcription factors are selected from the group consisting of: E2F Transcription Factor 3 (E2F3); Enhancer Of Zeste 2 Polycomb Repressive Complex 2 Subunit (EZH2); Homeobox protein DLX-6 (DLX6); Forkhead Box M1 (FOXM1); FOS Like 1, AP-1 Transcription Factor Subunit (FOSL1); Nuclear Factor Of Activated T Cells 4 (NFATC4); Myc proto-oncogene protein (MYC); Signal Transducer And Activator Of Transcription 4 (STAT4); GATA Binding Protein 3 (GATA3); Heat Shock Transcription Factor 2 (HSF2); Paired Box 4 (PAX4); NK2 Homeobox 2 (NKX2-2); SIM BHLH Transcription Factor 2 (SIM2); and Ventral Anterior Homeobox 1 (VAX1); and/or wherein the method comprising decreasing activity of one or more transcription factors in the cell, wherein the transcription factors are selected from the group consisting of: Signal Transducer And Activator Of Transcription 3 (STAT3); Zinc Finger Protein X-Linked (ZFX); Early growth response protein 1 (EGR1); Activating Transcription Factor 2 (ATF2); Vascular Endothelial Zinc Finger 1 (VEZF1); MYC Associated Zinc Finger Protein (MAZ); SRY-Box Transcription Factor 2 (SOX2); Paired Box 8 (PAX8); Zinc Finger Homeobox 3 (ZFHX3); Autophagy Related 4C Cysteine Peptidase (ATG4C); Autophagy Related 5 (ATG5); High Mobility Group Box 1 (HMGB1); GATA Binding Protein 2 (GATA2); and KLF Transcription Factor 4 (KLF4).

In some embodiments, the one or more transcription factors are selected from the group consisting of: STAT3, ZFX, EGR1, MAZ, SOX2, and ATF4. In some embodiments, the method comprises increasing activity of one or more transcription factors in the cell, wherein the one or more transcription factors are selected from the group consisting of E2F3, EZH2, FOXM1, and DLX6; and wherein the method comprises decreasing activity of one or more transcription factors in the cell, wherein the transcription factors are selected from the group consisting of STAT3, ZFX, EGR1, MAZ, SOX2, and ATF4. In some embodiments, the method comprises increasing activity of one or more transcription factors in the cell, wherein the one or more transcription factors are E2F3 or EZH2; and wherein the method comprises decreasing activity of one or more transcription factors in the cell, wherein the transcription factors are STAT3 or ZFX. In some embodiments, the method comprises increasing the activities of E2F3 and EZH2. In some embodiments, the method comprises decreasing the activities of STAT3 and ZFX. In some embodiments, the method comprises increasing the activity of E2F3 and decreasing the activity of STAT3. In some embodiments, the method comprises increasing the activity of E2F3 and decreasing the activity of ZFX. In some embodiments, the method comprises increasing the activity of EZH2 and decreasing the activity of STAT3. In some embodiments, the method comprises increasing the activities of EZH2 and decreasing the activity of ZFX. In some embodiments, the method comprises increasing the activities of E2F3 and EZH2 and decreasing the activity of STAT3. In some embodiments, the method comprises increasing the activities of E2F3 and EZH2 and decreasing the activity of ZFX. In some embodiments, the method comprises increasing the activity of EZH2 and decreasing the activities of STAT3 and ZFX. In some embodiments, the method comprises increasing the activities of E2F3 and EZH2 and decreasing the activities of STAT3 and ZFX.

In some embodiments, the activity of any two of the transcription factors is increased. In some embodiments, the activity of at least two of the transcription factors is increased. In some embodiments, the activity of any two of the transcription factors is decreased. In some embodiments, the activity of at least two of the transcription factors is decreased. In some embodiments, the activity of any two of the transcription factors is increased or decreased. In some embodiments, the activity of at least two of the transcription factors is increased.

### BRIEF DESCRIPTION OF THE DRAWINGS

FIGS. 1 A-1B show transcription factor (TF) perturbations can rejuvenate late passage skin fibroblasts back to an earlier passage state.

FIG. 1A shows a diagram of the high dimensional gene expression space indicating early passage, late passage, and perturbed late passage cells. Perturbed late passage cells that cluster close to early passage cells are considered to be “rejuvenated”. The rejuvenating transcription factor (TF) perturbations “move” the late passage cells along the direction of the rejuvenation vector, defined as the difference between early and late passage cells.

FIG. 1B shows the experimental set up. Late passage cells expressing CRISPRa (CRA) or CRISPRi (CRI) constructs were transfected with a sgRNA library targeting different TFs for activation or repression. The gene expression state of the transfected cells was then analyzed via Direct Capture Perturb-seq to quantify the mRNA expression and identify the sgRNA in each single cell. WT early and late passage cells were assayed in parallel to define the direction of aging (or its reverse, the direction of rejuvenation).

FIG. 2 shows correlation of global gene expression for perturbed late passage cells compared to WT passaged cells revealed top rejuvenating TFs.

FIG. 3 shows TF perturbations (rows) were clustered by the AUC t-test scores for selected TF modules (columns) from the SCENIC analysis (top panel). The bottom panel shows interaction network analysis (using string-db) of commonly up-regulated genes within a cluster of TF perturbations (CRA E2F3, CRA DLX6, CRI ZFX, CRI EGR1. CRI MAZ, CRI SOX2, and CRI ATF4). The lines connecting the circles indicate interactions or connections between genes. A group of TF perturbations drove similar gene regulatory networks, rewiring cells towards an earlier passage state.

FIG. 4 shows transcriptional signature of rejuvenation was shared across species, cell types, and rejuvenation methods. Each square indicates the AUC t-test score for a given TF module (column) derived from the comparison between two groups of cells (row).

FIGS. 5A-5C show TF perturbations increased cell division rates.

FIG. 5A shows KI67 microscopy of CRA NT and CRA E2F3 cells; 50 μM scale bar.

FIG. 5B shows Percent KI67 positive cells for CRA and CRI perturbed cells and WT passaged cells ranging from early to late population doubling (PD); N>1500 per sgRNA, and N>700 per WT PD; statistical significance is calculated by binomial distribution, relative to PD 9 for WT and NT for CRA and CRI. Data from experiments performed on different days was normalized and combined as described in exemplary methods. Similar data normalization was performed for other figures. Significance was calculated based on binomial distribution: *p<0.05, ** p<0.01, *** p<0.001.

FIG. 5C shows quantification of the percent of cells in S, G2, or M phase of the cell cycle, as measured via single cell RNA sequencing analysis for CRA and CRI perturbed cells and WT passaged cells ranging from early to late population doubling.

FIGS. 6A-6B show targeting E2F3 or EZH2 with CRA decreases total senescent cells.

FIG. 6A shows CRA NT and CRA E2F3 cells stained for beta-galactosidase (beta-gal), imaged in brightfield, 100 μM scale bar. Dark cells are beta-gal positive, which means they are senescent.

FIG. 6B shows quantification of percent beta-gal positive cells for CRA, CRI, and WT passaged cells; N>700 cells per sgRNA, N>400 for each WT PD. Statistical significance is calculated by binomial distribution, relative to NT for CRA and CRI and PD 9 for WT. * p<0.05, ** p<0.01, *** p<0.001.

FIG. 7A shows changes in the expression of p21 (CDKN1A) measured by qPCR for CRA and CRI is relative to NT controls, and the log 2fc for WT is relative to early passage cells.

FIG. 7B shows changes in the expression of TIMP1 measured by qPCR for CRA and CRI is relative to NT controls, and the log 2fc for WT is relative to early passage cells.

FIG. 7C shows changes in the expression of TIMP2 measured by qPCR for CRA and CRI is relative to NT controls, and the log 2fc for WT is relative to early passage cells

FIGS. 8A-8C shows improved proteostasis after TF perturbations.

FIG. 8A shows a Cluster heatmap of all proteasome genes, by log 2 fold change (log 2fc). In CRA and CRI cells, the log 2fc is relative to NT cells: for WT, the log 2fc is relative to early passage cells (WT PD 32 versus PD 14).

FIG. 8B shows proteasome activity for CRA and CRI TF perturbations and WT passaged cells; significance was calculated by a Wilcoxon rank-sum test, comparing TF perturbations to NT and WT later passages to PD 15. * p<0.05, ** p<0.01 *** p<0.001.

FIG. 8C shows CRI NT and CRI STAT3 cells stained with LysoTracker Red: 100 μM scale bar. Quantification of lysosome puncta per cell area is shown at right, as measured with LysoTracker Red. N>180 cells per sgRNA, and N>330 cells per WT PD. Significance was calculated by a Wilcoxon rank-sum test, comparing TF perturbations to NT and WT later PDs to WT PD 15. * p<0.05, ** p<0.01, *** p<0.001.

FIG. 9 A-9B shows TF perturbations enhanced mitochondrial function.

FIG. 9A shows a Cluster heatmap of mitochondrial genes (all “MT” mitochondrial genes except those encoding tRNA) and Krebs cycle genes, by log 2 fold change (log 2fc). In CRA and CRI cells, the log 2fc is relative to NT cells; for WT, the log 2fc is relative to early passage cells (WT PD 32 versus PD 14).

FIG. 9B shows TMRE (tetramethylrhodamine, ethyl ester) mitochondrial membrane potential stain of CRA NT and CRA EZH2 cells; higher intensity indicates higher membrane potential; scale bar is 100 μM. Quantification of the TMRE membrane potential stain is shown at right. Significance was calculated by a Wilcoxon rank-sum test, comparing TF perturbations to NT and WT later passages to WT PD 15. N>300 cells per sgRNA, N>465 cells per WT PD. * p<0.05, ** p<0.01, *** p<0.001.

FIGS. 10A-10B shows total γH2AX DNA foci do not change considerably in TF perturbations, but there is an increase in late passage WT cells.

FIG. 10A shows CRI NT γH2AX DNA foci imaging; 50 μM scale bar.

FIG. 10B shows Quantification of γH2AX puncta per cell; N>500 cells per TF perturbation, N>240 cells for each WT PD. Significance was calculated by a Wilcoxon rank-sum test, comparing TF to NT for CRA and CRI, and later passages to PD 9 for WT. * p<0.05, ** p<0.01, *** p<0.001.

FIGS. 11A-11D show total expression of epigenetic markers H3K9me3 and H3K27me3 are variable across TF perturbations but trend upwards in later WT passages.

FIG. 11A shows CRI NT and CRI ZFX H3K9me3 staining; 5 μM scale bar.

FIG. 11B shows quantification of H3K9me3 total fluorescence per nucleus. For CRA and CRI cells, N>250 cells per sgRNA and N>1,170 cells per WT PD; significance was calculated by a Wilcoxon rank-sum test compared to NT for CRA and CRI, and PD 15 for WT. * p<0.05, ** p<0.01, *** p<0.001.

FIG. 11C shows CRA NT and CRA EZH2 H3K27me3 staining: 50 μM scale bar.

FIG. 11D shows quantification of H3K27me3 total fluorescence per nucleus. For CRA and CRI cells, N>250 cells per sgRNA and N>600 cells per WT PD; significance was calculated by a Wilcoxon rank-sum test compared to NT for CRA and CRI, and PD 15 for WT. * p<0.05, ** p<0.01, *** p<0.001.

FIG. 12 shows skin methylation clock analysis of WT passaged cells.

FIGS. 13A-13B show TF perturbations and their effects on late passage cells are independent of telomeres.

FIG. 13A shows differentially expressed genes when TERT was overexpressed in human skin fibroblasts. In CRA and CRI cells, the log 2fc is relative to NT cells; for TERT overexpression, the log 2fc is relative to untransformed primary fibroblasts.

FIG. 13B shows Relative telomere length, as determined through qPCR analysis, for WT passaged cells and CRA and CRI TF perturbations.

FIGS. 14A-14C shows TF perturbations did not lead to cancer-like gene expression.

FIG. 14A shows differentially expressed genes when SV40 large-t antigen was overexpressed in human skin fibroblasts. In CRA and CRI cells, the log two fold change (log 2fc) is relative to NT cells; for SV40 cells, the log 2fc is relative to TERT overexpressing fibroblasts.

FIG. 14B shows differentially expressed genes when oncogenic H-Ras (RASG12V) was introduced to human skin. In CRA and CRI cells, the log 2fc is relative to NT cells; for Ras cells overexpression, the log 2fc is relative to the SV40 cells.

FIG. 14C shows commonly differentially expressed genes from seven cancer types. In CRA and CRI cells, the log 2fc is relative to NT cells.

FIG. 15 shows top 14 CRISPRa (CRA) TF perturbations, ranked by r value.

FIG. 16 shows top 14 CRISPRi (CRI) TF perturbations, ranked by r value.

FIG. 17 shows qPCR data demonstrating that E2F3 and EZH2 are overexpressed when targeted with an overexpression plasmid in primary patient-derived fibroblasts. The numbers (63, 73, and 85) indicate the age in years of the patient from which the fibroblasts originate.

FIG. 18 shows qPCR demonstrating that STAT3 and ZFX are repressed when targeted with siRNA in primary patient-derived fibroblasts. The numbers (63, 73, 85, and 89) indicate the age in years of the patient from which the fibroblasts originate.

FIG. 19 shows proteosome activity data in primary patient-derived fibroblasts transfected with plasmid for expressing E2F3 or EZH2 or with siRNA repressing expression of STAT3 or ZFX. The numbers (63, 73, 85, and 89) indicate the age in years of the patient from which the fibroblasts originate. The y axis shows the proteasome activity for each treatment group.

FIG. 20 shows K167 expression data in primary patient-derived fibroblasts transfected with plasmid for expressing E2F3 or EZH2 or with siRNA repressing expression of STAT3 or ZFX. The y-axis is shown in fold change in KI67+cells relative to the control (GFP for E2F3 and EZH2; NT siRNA for ZFX and STAT3). E2F3 is shown on a separate plot to make visualization easier. The numbers (63, 73, 85, and 89) indicate the age in years of the patient from which the fibroblasts originate.

FIG. 21 shows qPCR data for the expression of each targeted TF and two senescence-marker genes, TIMP1 and p21 in late passage skin fibroblasts transfected with plasmid for expressing E2F3 or EZH2 or with siRNA repressing expression of STAT3 or ZFX.

FIG. 22 show quantification of K167 expression in late passage skin fibroblasts transfected with plasmid for expressing E2F3 or EZH2 or with siRNA repressing expression of STAT3 or ZFX.

FIG. 23 shows quantification of proteasome activity in late passage skin fibroblasts transfected with plasmid for expression E2F3 or EZH2 or with siRNA repressing expression of STAT3 or ZFX. Three of the four TFs lead to increased proteasome activity in late passage cells.

FIG. 24A shows data demonstrating decreased expression of p21, a senescence gene, following repression of EGR1 via CRISPRi. Log2fc means log base two-fold change, where a negative one means that the gene is 50% less expressed in that condition than in the control.

FIG. 24B shows quantification of KI67 expression following repression of EGR1 via CRISPRi. A higher percentage of cells that are K167+ means there are more cells actively dividing. This higher rate is an indication that cells are behaving younger and healthier. CRI means EGR1 is repressed using CRISPRi. NT refers to non-targeting control cells.

FIG. 24C shows quantification of proteasome activity following repression of EGR1 via CRISPRi. CRI means EGR1 is repressed using CRISPRi. NT refers to non-targeting control cells. Higher proteasome activity is an indication that cells are behaving younger and healthier. Proteasome activity is increased upon repression of EGR1.

FIG. 24D shows number of lysosomes per cell area following repression of EGR1 via CRISPRi. CRI means EGR1 is repressed using CRISPRi. NT refers to non-targeting control cells.

FIG. 25 shows violin plots of relative expression of common fibroblast marker genes following perturbations of TFs. The y-axis is the relative expression of each gene and the name of the gene being shown is written along the y-axis. Here the relative expression of VIM, COL1A2, COL1A1, and S100A4 are shown. CRISPRa was performed for E2F3 and EZH2, and CRISPRi for STAT3 and ZFX; PD #stands for population doubling. A higher number is a later passage cell.

FIG. 26A shows quantification of senescent cells by beta-galactosidase assay following overexpression of FOXM1 via CRISPRa. CRA FOXM1 means FOXM1 is overexpressed using CRISPRa. NT refers to nontargeting control cells. Overexpressing FOXM1 leads to rejuvenation phenotypes.

FIG. 26B shows quantification of lysosomal puncta per cell area using LysoTracker staining following overexpression of FOXM1 via CRISPRa. CRA FOXM1 means FOXM1 is overexpressed using CRISPRa. NT refers to nontargeting control cells.

FIG. 26C shows quantification of relative gene expression of p21, a senescence gene, using qPCR following overexpression of FOXM1 via CRISPRa. Data is shown relative to NT cells (nontargeting control cells).

FIG. 27A shows quantification of KI67+ cells following overexpression of the Yamanaka factors (OCT4, SOX2, KLF4, MYC) using an overexpression plasmid. OSKM indicates cells which received the overexpression plasmid for the Yamanaka factors.

FIG. 27B shows quantification of gene expression via qPCR of the Yamanaka factors as well as K167 and p21, a senescence gene, in cells overexpressing the Yamanaka factors.

FIG. 27C shows quantification of proteasome activity following overexpression of the Yamanaka factors (OCT4, SOX2, KLF4, MYC) using an overexpression plasmid. OSKM indicates cells which received the overexpression plasmid for the Yamanaka factors.

FIG. 27D shows quantification of lysosomal puncta per cell area following overexpression of the Yamanaka factors (OCT4, SOX2, KLF4, MYC) using an overexpression plasmid. OSKM indicates cells which received the overexpression plasmid for the Yamanaka factors.

FIG. 27E shows quantification of mitochondrial membrane potential via TMRE staining following overexpression of the Yamanaka factors (OCT4, SOX2, KLF4, MYC) using an overexpression plasmid. OSKM indicates cells which received the overexpression plasmid for the Yamanaka factors.

FIG. 28 shows correlation data from RNA sequencing of livers of aged mice overexpressing EZH2. Here, the log 2fc of all genes is shown in young mice vs aged mice (both with the control GFP overexpression) on the x-axis and the log 2fc of all genes in aged mice overexpressing EZH2 vs to aged mice overexpressing the control (GFP) on the y-axis. A gene shows reversal back to a younger state if the log 2fc correlate directly with each group (ex: highly expressed in young mice, and highly in aged mice with EZH2 overexpressed).

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the fibroblast” includes reference to one or more fibroblast and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific embodiments, the terms refer to a polymeric form of amino acids of any length which include genetically coded amino acids. In particular embodiments, the terms refer to a polymeric form of amino acids of any length which include genetically coded amino acids fused to a heterologous amino acid sequence.

The term “heterologous” refers to two components that are defined by structures derived from different sources. For example, “heterologous” polynucleic acids include expression constructs in which a polynucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both).

The term “operably linked” refers to linkage between molecules to provide a desired function. By way of example, a polynucleic acid expression control sequence (such as a promoter, signal sequence, or array of transcription factor binding sites) may be operably linked to a second polynucleotide, wherein the expression control sequence affects transcription and/or translation of the second polynucleotide.

An “exogenous” molecule is a molecule that is not normally present in a cell but can be introduced into a cell by one or more genetic, biochemical or other methods. An exogenous molecule can comprise, for example, a protein that is not expressed by the cell or expressed at low or an undetectable level. Thus, in certain embodiments, the exogenous molecule may be identical in sequence to the endogenous molecule. An exogenous polynucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

The terms “treat”, “treating”, treatment” and the like refer to a course of action initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

As used herein, the term “rejuvenating”, “rejuvenation” and “rejuvenate” in the context of cells refers to decreasing the phenotypic, physiological, and/or genetic age of the cells. Non-limiting examples of phenotypic, physiological, and/or genetic age of the cells can include cell division, cell proliferation, cellular senescence, epigenetic age, proteasome activity, metabolism, or mitochondrial function. In some embodiments, the rejuvenated cells have features closer to that of younger cells as compared to unrejuvenated control cells. Younger cells have certain features that are distinguishable from older cells. For example, younger cells divide more, have less senescence, have higher proteasome activity, have fewer senescence-associated lysosomes, have higher mitochondrial gene expression, have higher metabolism gene expression and/or higher mitochondrial membrane potential than older cells, have a decreased methylation age as determined using DNA methylation clock analysis, have gene expression and phenotypes more similar to early passage states.

As used herein, the term “unrejuvenated cell(s)” or “unrejuvenated control cell(s)” refers to cells that have not been subjected to the methods for rejuvenating disclosed herein.

As used herein, the term “E2F3” refers to the transcription factor E2F Transcription Factor 3 and encompasses proteins that have at least 80% amino acid sequence identity to the amino acid sequence set forth as UniProt Accession No. 000716 and has E2F3 activity of binding to regulatory region of the genes activated by E2F3 and activating gene expression. In certain embodiments, the E2F3 is a human E2F3. In certain embodiments, the E2F3 is a mouse E2F3 or a non-human primate E2F3.

As used herein, the term “EZH2” refers to the transcription factor Enhancer Of Zeste 2 Polycomb Repressive Complex 2 Subunit or Histone-lysine N-methyltransferase EZH2 and encompasses proteins that have at least 80% amino acid sequence identity to the amino acid sequence set forth as UniProt Accession No. Q15910 and has EZH2 activity. In certain embodiments, the EZH2 is a human EZH2. In certain embodiments, the EZH2 is a mouse EZH2 or a non-human primate EZH2.

As used herein, the term “DLX6” refers to the transcription factor Homeobox protein DLX-6 and encompasses proteins that have at least 80% amino acid sequence identity to the amino acid sequence set forth as UniProt Accession No. P56179 and has DLX6 activity. In certain embodiments, the DLX6 is a human DLX6. In certain embodiments, the DLX6 is a mouse DLX6 or a non-human primate DLX6.

As used herein, the term “FOXM1” refers to the transcription factor Forkhead Box M1 and encompasses proteins that have at least 80% amino acid sequence identity to the amino acid sequence set forth as UniProt Accession No. Q08050 and has FOXM1 activity. In certain embodiments, the FOXM1 is a human FOXM1. In certain embodiments, the EZH2 is a mouse FOXM1 or a non-human primate FOXM1.

As used herein, the term “FOSL1” refers to the transcription factor FOS Like 1, AP-1 Transcription Factor Subunit and encompasses proteins that have at least 80% amino acid sequence identity to the amino acid sequence set forth as UniProt Accession No. P15407 and has FOSL1 activity. In certain embodiments, the FOSL1 is a human FOSL1. In certain embodiments, the FOSL1 is a mouse FOSL1 or a non-human primate FOSL1.

As used herein, the term “NFATC4” refers to the transcription factor Nuclear Factor Of Activated T Cells 4 and encompasses proteins that have at least 80% amino acid sequence identity to the amino acid sequence set forth as UniProt Accession No. Q14934 and has NFATC4 activity. In certain embodiments, the NFATC4 is a human NFATC4. In certain embodiments, the NFATC4 is a mouse NFATC4 or a non-human primate NFATC4.

As used herein, the term “MYC” refers to the transcription factor Myc proto-oncogene protein and encompasses proteins that have at least 80% amino acid sequence identity to the amino acid sequence set forth as UniProt Accession No. P01106 and has MYC activity. In certain embodiments, the MYC is a human MYC. In certain embodiments, the MYC is a mouse MYC or a non-human primate MYC.

As used herein, the term “STAT4” refers to the transcription factor Signal Transducer And Activator Of Transcription 4 and encompasses proteins that have at least 80% amino acid sequence identity to the amino acid sequence set forth as UniProt Accession No. Q14765 and has STAT4 activity. In certain embodiments, the STAT4 is a human STAT4. In certain embodiments, the STAT4 is a mouse STAT4 or a non-human primate STAT4.

As used herein, the term “GATA3” refers to the transcription factor GATA Binding Protein 3 and encompasses proteins that have at least 80% amino acid sequence identity to the amino acid sequence set forth as UniProt Accession No. P23771 and has GATA3 activity. In certain embodiments, the GATA3 is a human GATA3. In certain embodiments, the GATA3 is a mouse GATA3 or a non-human primate GATA3.

As used herein, the term “HSF2” refers to the transcription factor Heat Shock Transcription Factor 2 and encompasses proteins that have at least 80% amino acid sequence identity to the amino acid sequence set forth as UniProt Accession No. Q03933 and has HSF2 activity. In certain embodiments, the HSF2 is a human HSF2. In certain embodiments, the HSF2 is a mouse HSF2 or a non-human primate HSF2.

As used herein, the term “PAX4” refers to the transcription factor Paired Box 4 and encompasses proteins that have at least 80% amino acid sequence identity to the amino acid sequence set forth as UniProt Accession No. 043316 and has PAX4 activity. In certain embodiments, the PAX4 is a human PAX4. In certain embodiments, the PAX4 is a mouse PAX4 or a non-human primate PAX4.

As used herein, the term “NKX2-2” refers to the transcription factor NK2 Homeobox 2 and encompasses proteins that have at least 80% amino acid sequence identity to the amino acid sequence set forth as UniProt Accession No. 095096 and has NKX2-2 activity. In certain embodiments, the NKX2-2 is a human NKX2-2. In certain embodiments, the NKX2-2 is a mouse NKX2-2 or a non-human primate NKX2-2.

As used herein, the term “SIM2” refers to the transcription factor SIM BHLH Transcription Factor 2 and encompasses proteins that have at least 80% amino acid sequence identity to the amino acid sequence set forth as UniProt Accession No. Q14190 and has SIM2 activity. In certain embodiments, the SIM2 is a human SIM2. In certain embodiments, the SIM2 is a mouse SIM2 or a non-human primate SIM2.

As used herein, the term “VAX1” refers to the transcription factor Ventral Anterior Homeobox 1 and encompasses proteins that have at least 80% amino acid sequence identity to the amino acid sequence set forth as UniProt Accession No. Q5SQQ9 and has VAX1 activity. In certain embodiments, the VAX1 is a human VAX1. In certain embodiments, the VAX1 is a mouse VAX1 or a non-human primate VAX1.

As used herein, the term “STAT3” refers to the transcription factor Signal Transducer And Activator Of Transcription 3 and encompasses proteins that have at least 80% amino acid sequence identity to the amino acid sequence set forth as UniProt Accession No. P40763 and has STAT3 activity. In certain embodiments, the STAT3 is a human STAT3. In certain embodiments, the STAT3 is a mouse STAT3 or a non-human primate STAT3.

As used herein, the term “ZFX” refers to the transcription factor Zinc Finger Protein X-Linked and encompasses proteins that have at least 80% amino acid sequence identity to the amino acid sequence set forth as UniProt Accession No. P17010 and has ZFX activity. In certain embodiments, the ZFX is a human ZFX. In certain embodiments, the ZFX is a mouse ZFX or a non-human primate ZFX.

As used herein, the term “EGR1” refers to the transcription factor Early growth response protein 1 and encompasses proteins that have at least 80% amino acid sequence identity to the amino acid sequence set forth as UniProt Accession No. P18146 and has EGR1 activity. In certain embodiments, the EGR1 is a human EGR1. In certain embodiments, the EGR1 is a mouse EGR1 or a non-human primate EGR1.

As used herein, the term “ATF2” refers to the transcription factor Activating Transcription Factor 2 and encompasses proteins that have at least 80% amino acid sequence identity to the amino acid sequence set forth as UniProt Accession No. P15336 and has ATF2 activity. In certain embodiments, the ATF2 is a human ATF2. In certain embodiments, the ATF2 is a mouse ATF2 or a non-human primate ATF2.

As used herein, the term “VEZF1” refers to the transcription factor Vascular Endothelial Zinc Finger 1 and encompasses proteins that have at least 80% amino acid sequence identity to the amino acid sequence set forth as UniProt Accession No. Q14119 and has VEZF1 activity. In certain embodiments, the VEZF1 is a human VEZF1. In certain embodiments, the VEZF1 is a mouse VEZF1 or a non-human primate VEZF1.

As used herein, the term “MAZ” refers to the transcription factor MYC Associated Zinc Finger Protein and encompasses proteins that have at least 80% amino acid sequence identity to the amino acid sequence set forth as UniProt Accession No. P56270 and has MAZ activity. In certain embodiments, the MAZ is a human MAZ. In certain embodiments, the MAZ is a mouse MAZ or a non-human primate MAZ.

As used herein, the term “SOX2” refers to the transcription factor SRY-Box Transcription Factor 2 and encompasses proteins that have at least 80% amino acid sequence identity to the amino acid sequence set forth as UniProt Accession No. P48431 and has SOX2 activity. In certain embodiments, the SOX2 is a human SOX2. In certain embodiments, the SOX2 is a mouse SOX2 or a non-human primate SOX2.

As used herein, the term “PAX8” refers to the transcription factor Paired Box 8 and encompasses proteins that have at least 80% amino acid sequence identity to the amino acid sequence set forth as UniProt Accession No. Q06710 and has PAX8 activity. In certain embodiments, the PAX8 is a human PAX8. In certain embodiments, the PAX8 is a mouse PAX8 or a non-human primate PAX8.

As used herein, the term “ZFHX3” refers to the transcription factor Zinc Finger Homeobox 3 and encompasses proteins that have at least 80% amino acid sequence identity to the amino acid sequence set forth as UniProt Accession No. Q15911 and has ZFHX3 activity. In certain embodiments, the ZFHX3 is a human ZFHX3. In certain embodiments, the ZFHX3 is a mouse ZFHX3 or a non-human primate ZFHX3.

As used herein, the term “ATG4C” refers to the transcription factor Autophagy Related 4C Cysteine Peptidase and encompasses proteins that have at least 80% amino acid sequence identity to the amino acid sequence set forth as UniProt Accession No. Q96DT6 and has ATG4C activity. In certain embodiments, the ATG4C is a human ATG4C. In certain embodiments, the ATG4C is a mouse ATG4C or a non-human primate ATG4C.

As used herein, the term “ATG5” refers to the transcription factor Autophagy Related 5 and encompasses proteins that have at least 80% amino acid sequence identity to the amino acid sequence set forth as UniProt Accession No. Q9H1Y0 and has ATG5 activity. In certain embodiments, the ATG5 is a human ATG5. In certain embodiments, the ATG5 is a mouse ATG5 or a non-human primate ATG5.

As used herein, the term “HMGB1” refers to the transcription factor High Mobility Group Box 1 and encompasses proteins that have at least 80% amino acid sequence identity to the amino acid sequence set forth as UniProt Accession No. P09429 and has HMGB1 activity. In certain embodiments, the HMGB1 is a human HMGB1. In certain embodiments, the HMGB1 is a mouse HMGB1 or a non-human primate HMGB1.

As used herein, the term “GATA2” refers to the transcription factor GATA Binding Protein 2 and encompasses proteins that have at least 80% amino acid sequence identity to the amino acid sequence set forth as UniProt Accession No. P23769 and has GATA2 activity. In certain embodiments, the GATA2 is a human GATA2. In certain embodiments, the GATA2 is a mouse GATA2 or a non-human primate GATA2.

As used herein, the term “KLF4” refers to the transcription factor KLF Transcription Factor 4 and encompasses proteins that have at least 80% amino acid sequence identity to the amino acid sequence set forth as UniProt Accession No. 043474 and has KLF4 activity. In certain embodiments, the KLF4 is a human KLF4. In certain embodiments, the KLF4 is a mouse KLF4 or a non-human primate KLF4.

### Methods for Rejuvenating Cells

Methods and compositions for rejuvenating cells are provided herein. In certain aspects, the method includes increasing activity of one or more of transcription factors (TFs). In certain aspects, the method includes increasing activity of one or more of E2F Transcription Factor 3 (E2F3), Enhancer Of Zeste 2 Polycomb Repressive Complex 2 Subunit (EZH2), Homeobox protein DLX-6 (DLX6), Forkhead Box M1 (FOXM1), FOS Like 1, AP-1 Transcription Factor Subunit (FOSL1), Nuclear Factor Of Activated T Cells 4 (NFATC4), Myc proto-oncogene protein (MYC), Signal Transducer And Activator Of Transcription 4 (STAT4), GATA Binding Protein 3 (GATA3), Heat Shock Transcription Factor 2 (HSF2), Paired Box 4 (PAX4), NK2 Homeobox 2 (NKX2-2), SIM BHLH Transcription Factor 2 (SIM2), and Ventral Anterior Homeobox 1 (VAX1) in the cells.

In certain aspects, the method includes decreasing the activity of one or more TFs. In certain aspects, the method includes decreasing the activity of one or more of Signal Transducer And Activator Of Transcription 3 (STAT3), Zinc Finger Protein X-Linked (ZFX), Early growth response protein 1 (EGR1), Activating Transcription Factor 2 (ATF2), Vascular Endothelial Zinc Finger 1 (VEZF1), MYC Associated Zinc Finger Protein (MAZ), SRY-Box Transcription Factor 2 (SOX2), Paired Box 8 (PAX8), Zinc Finger Homeobox 3 (ZFHX3), Autophagy Related 4C Cysteine Peptidase (ATG4C), Autophagy Related 5 (ATG5), High Mobility Group Box 1 (HMGB1), GATA Binding Protein 2 (GATA2), and KLF Transcription Factor 4 (KLF4) in the cells.

In certain aspects, the method includes increasing activity of one or more TFs and decreasing activity of one or more TFs simultaneously. In certain aspects, the method includes increasing activity of one or more TFs and decreasing activity of one or more TFs sequentially. In certain aspects, the increasing activity of one or more TFs occur prior to the decreasing activity of one or more TFs. In certain aspects, the decreasing activity of one or more TFs occur prior to the increasing activity of one or more TFs.

In certain aspects, the method includes increasing the activity of one or more of E2F Transcription Factor 3 (E2F3), Enhancer Of Zeste 2 Polycomb Repressive Complex 2 Subunit (EZH2), Homeobox protein DLX-6 (DLX6), Forkhead Box M1 (FOXM1), FOS Like 1, AP-1 Transcription Factor Subunit (FOSL1), Nuclear Factor Of Activated T Cells 4 (NFATC4), Myc proto-oncogene protein (MYC), Signal Transducer And Activator Of Transcription 4 (STAT4), GATA Binding Protein 3 (GATA3), Heat Shock Transcription Factor 2 (HSF2), Paired Box 4 (PAX4), NK2 Homeobox 2 (NKX2-2), SIM BHLH Transcription Factor 2 (SIM2), and Ventral Anterior Homeobox 1 (VAX1) in the cells and decreasing the activity of one or more of Signal Transducer And Activator Of Transcription 3 (STAT3), Zinc Finger Protein X-Linked (ZFX), Early growth response protein 1 (EGR1), Activating Transcription Factor 2 (ATF2), Vascular Endothelial Zinc Finger 1 (VEZF1), MYC Associated Zinc Finger Protein (MAZ), SRY-Box Transcription Factor 2 (SOX2), Paired Box 8 (PAX8), Zinc Finger Homeobox 3 (ZFHX3), Autophagy Related 4C Cysteine Peptidase (ATG4C), Autophagy Related 5 (ATG5), High Mobility Group Box 1 (HMGB1), GATA Binding Protein 2 (GATA2), and KLF Transcription Factor 4 (KLF4) in the cells.

In certain aspects, the method includes increasing activity of one or both of E2F3 and EZH2 and/or decreasing the activity of one, two or three of STAT3, ZFX, and EGR1 in the cells.

In certain aspects, the method includes increasing activity of one or both of E2F3 and EZH2 in the cells. In certain embodiments, the method includes decreasing the activity of STAT3 and ZFX in the cells. In certain embodiments, the method includes increasing activity of one or both of E2F3 and EZH2 and decreasing the activity of one or both of STAT3 and ZFX in the cells. In certain aspects, the method includes increasing activity of only E2F3 in the cells. In certain aspects, the method includes increasing activity of only EZH2 in the cells. In certain embodiments, the method includes decreasing the activity of only STAT3 in the cells. In certain embodiments, the method includes decreasing the activity of only ZFX in the cells.

In some embodiments, a rejuvenated cell has higher rate of cell division, less senescence, higher proteasome activity, fewer senescence-associated lysosomes, higher mitochondrial gene expression, higher metabolism gene expression, higher metabolism, higher ribosome biogenesis, higher expression of cell cycle-related genes, or higher mitochondrial membrane potential than an un-rejuvenated cell.

In some embodiments, a rejuvenated cell is less differentiated or more pluripotent than an un-rejuvenated cell. Differentiation can be determined by any suitable marker.

In some embodiments, a rejuvenated cell has a decreased methylation age as determined using DNA methylation clock analysis than an un-rejuvenated cell.

In some embodiments, a rejuvenated cell has increased self-renewal ability than an unrejuvenated cell.

In some embodiments, the method of rejuvenation provided herein includes transforming an old cell from a subject to acquire traits of a young cell from a subject biologically, phenotypically, physiologically, transcriptomically, proteomically, epigenetically or genetically.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar biologically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar phenotypically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar physiologically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar transcriptomically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar proteomically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar epigenomically to a young cell than to an old cell.

In some embodiments, a rejuvenated cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more similar genetically to a young cell than to an old cell.

In some embodiments, similarity between a rejuvenated cell and a young cell, similarity between a rejuvenated cell and an old cell, or similarity between a rejuvenated cell and an un-rejuvenated cell can be determined using any suitable method, such as principal component analysis based on sequencing data. The sequencing data can be data acquired using any next-generation sequencing method. In some embodiments, difference between a rejuvenated cell and a young cell, difference between a rejuvenated cell and an old cell, or difference between a rejuvenated cell and an un-rejuvenated cell can be determined using any suitable method, such as principal component analysis based on sequencing data. The sequencing data can be data acquired using any next-generation sequencing method. Non-limiting examples of next-generation sequencing include whole-genome sequencing, whole-exome sequencing, targeted gene sequencing, single-cell RNAseq, bulk RNAseq, total RNA-seq, gene expression profiling, iRNA and small RNA analysis, ChiP-seq, methylation sequencing, 16S metagenomic sequencing, shotgun metagenomics, metatranscriptomics, cell-free sequencing, or liquid biopsy analysis.

In some embodiments, an old cell is a cell from a subject at least 20 years old. For example, an old cell can be a cell from a subject that is at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or at least 100 years old.

In some embodiments, a young cell is a cell from a subject at most 30 years old. For example, a young cell can be a cell from a subject that is at most 3, 4, 5, 6, 7, 8, 9, 10, 11, 12, 13, 14, 15, 16, 17, 18, 19, 20, 21, 22, 23, 24, 25, 26, 27, 28, or at most 29 years old.

In some embodiments, the cell is a young cell. In some embodiments, the cell is an old cell. Age of a cell can be determined by any suitable proliferation marker (i.e., K167) or any suitable senescence marker (i.e., p21, p16). In some embodiments, a young cell has been cultured in vitro or ex vivo for less than 30 passages. For example, a young cell can be a cell that has been cultured in vitro or ex vivo for less than 1, 2, 3, 4, 5, 6, 7, 8, 9, 10, 11, 12, 13, 14, 15, 16, 17, 18, 19, 20, 21, 22, 23, 24, 25, 26, 27, 28, or less than 29 passages.

In some embodiments, an old cell has been cultured in vitro or ex vivo for more than 30 passages. For example, an old cell can be a cell that has been cultured in vitro or ex vivo for more than 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or more than 100 passages.

In some embodiments, the cell has been cultured in vitro or ex vivo for at least 1 passage. For example, in some embodiments, the cell has been cultured in vitro or ex vivo for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 at least 100 passages.

In some embodiments, the cell has been cultured in vitro or ex vivo for at most 100 passages. For example, in some embodiments, the cell has been cultured in vitro or ex vivo for at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, at most 99 passages.

In certain embodiments, a rejuvenated cell has a rate of cell division similar to a young cell. For example, the rejuvenated cell divides more than the unrejuvenated control cells. Cell division can be measured by any suitable method, such as quantification of expression levels (i.e., mRNA, protein) of any one or more suitable cell division markers. Non-limiting methods of determining cell division can include immunofluorescent imaging, immunohistochemistry, flow cytometry, qPCR, or next generation sequencing. Cell division markers can include but are not limited to K167 and phosphohistone H3. In some embodiments, cell division can be determined by Edu pulse-chase experiment or Brdu pulse-chase experiment. In some embodiments, cell division can be determined by measuring the number of cells or number of population doublings.

In some embodiments, a rejuvenated cell population has more cells in G1 phase than cells in an unrejuvenated cell population. In some embodiments, a rejuvenated cell population has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more cells in G1 phase than cells in an unrejuvenated cell population.

In some embodiments, a rejuvenated cell population has more cells in S phase than cells in an unrejuvenated cell population. In some embodiments, a rejuvenated cell population has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more cells in S phase than cells in an unrejuvenated cell population.

In some embodiments, a rejuvenated cell population has more cells in G2 phase than cells in an unrejuvenated cell population. In some embodiments, a rejuvenated cell population has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more cells in G2 phase than cells in an unrejuvenated cell population.

In some embodiments, a rejuvenated cell population has more cells in M phase than cells in an unrejuvenated cell population. In some embodiments, a rejuvenated cell population has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more cells in M phase than cells in an unrejuvenated cell population.

In some embodiments, a unrejuvenated cell population has more cells in G0 phase than cells in a rejuvenated cell population. In some embodiments, an unrejuvenated cell population has at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80% more cells in G0 phase than cells in a rejuvenated cell population.

In some embodiments, a rejuvenated cell a has higher proliferation rate than an unrejuvenated control cell. Cell proliferation can be measured by any suitable method, such as quantification of expression levels (i.e., mRNA, protein) of any one or more suitable cell proliferation markers. Exemplary methods can include immunofluorescent imaging, immunohistochemistry, flow cytometry, qPCR, or next generation sequencing. Cell proliferation markers can include, but are not limited to Cyclin D1, Cyclin E, Cyclin A, Cyclin B, CDK2, CDK4/6, Cdk1, or pRb. In some embodiments, cell proliferation can be determined by Edu pulse-chase experiment or Brdu pulse-chase experiment.

Control cells can have the same age as the cells being rejuvenated. In certain embodiments, the rejuvenated cells have less senescence than unrejuvenated control cells. Senescence can be measured by any suitable method, such as quantification of expression levels (e.g., mRNA, protein) of any one or more suitable senescence markers. Exemplary methods can include immunofluorescent imaging, immunohistochemistry, flow cytometry, qPCR, or next generation sequencing. Senescence markers can include but are not limited to p21, TIMP1, TIMP2 or p16. In some embodiments, senescence can be determined by Edu pulse-chase experiment or Brdu pulse-chase experiment. In some embodiments, senescence can be measured by γH2AX puncta via immunofluorescent imaging. In some embodiments, senescence can be measured by number of cells or number of population doublings.

In certain embodiments, the rejuvenated cells have increased proteasome activity as compared to unrejuvenated control cells. Proteasome activity can be measured by a fluorescence-based assay wherein a substrate fluoresces following proteasome-mediated cleavage. In certain embodiments, the rejuvenated cells have decreased senescence-associated lysosomes as compared to unrejuvenated control cells as measured by quantification of lysosomal puncta following LysoTracker™ staining. In certain embodiments, the rejuvenated cells have increased mitochondrial and metabolism gene expression. Exemplary mitochondrial and metabolism genes which may have increased expression include one or more of MT-ATP6, MT-ATP8, MT-CYB, MT-CO1, MT-CO2, MT-CO3, MT-ND4L, MT-ND1, MT-ND2, MT-ND3, MT-ND4, MT-ND5, MT-ND6, ACLY, ACO1, ACO2, CS, DLAT, DLD, DLST, FH, IDH1, IDH2, IDH3A, IDH3B, IDH3G, MDH1, MDH2, OGDH, OGDHL, PC, PCK1, PCK2, PDHA1, PDHA2, PDHB, SDHA, SDHB, SDHC, SDHD, SUCLA2, SUCLG1, and SUCLG2. In certain embodiments, the rejuvenated cells have increased mitochondrial membrane potential as compared to unrejuvenated control cells as measured by quantification of fluorescence following staining with membrane potential indicator TMRE (tetramethylrhodamine, ethyl ester). In some embodiments, the rejuvenated cells have increased metabolism as compared to unrejuvenated control cells. Non-limiting examples of methods to measure metabolism include SeaHorse real-time cell metabolic analysis, glucose/glucose oxidase assay, galactose/galactose oxidase assay, uric acid/uricase assay, glycolysis assay, fatty acid metabolism assay, ATP detection assay, ADP/ATP ratio assay, phosphate assay, NAD/NADH assay, NADP/NADPH assay, or NADPH assay.

In certain embodiments, the method disclosed herein may result at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more increase in cell division as compared to control cells not subjected to the method.

In certain embodiments, the method disclosed herein may result in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more decrease in senescence as compared to control cells not subjected to the method.

In certain embodiments, the method disclosed herein may result in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more increase in proteasome activity as compared to control cells not subjected to the method.

In certain embodiments, the method disclosed herein may result in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more decrease in senescence-associated lysosomes as compared to control cells not subjected to the method.

In certain embodiments, the method disclosed herein may result in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more increase in mitochondrial and metabolism gene expression as compared to control cells not subjected to the method. Exemplary mitochondrial and metabolism genes which may have increased expression include one or more of MT-ATP6, MT-ATP8, MT-CYB, MT-CO1, MT-CO2, MT-CO3, MT-ND4L, MT-ND1, MT-ND2, MT-ND3, MT-ND4, MT-ND5, MT-ND6, ACLY, ACO1, ACO2, CS, DLAT, DLD, DLST, FH, IDH1, IDH2, IDH3A, IDH3B, IDH3G, MDH1, MDH2, OGDH, OGDHL, PC, PCK1, PCK2, PDHA1, PDHA2, PDHB, SDHA, SDHB, SDHC, SDHD, SUCLA2, SUCLG1, and SUCLG2.

In certain embodiments, the method disclosed herein may result in at least 5%, at least 10%, at least 15%, at least 20%, at least 25%, or more increase in mitochondrial function as compared to control cells not subjected to the method. In some embodiments, mitochondrial functions can be determined by gene expression or protein expression levels of mitochondrial and Krebs cycle genes. Mitochondrial function can be measured by any suitable methods, such as measuring membrane potential (i.e., TMRE membrane potential marker), superoxide production, calcium levels, mitochondrial permeability, ATP production, or NAD/NADH levels.

In certain embodiments, the method disclosed herein may result in at least 5%, at least 10%, at least 15%, at least 20%, at least 25%, or more increase in mitochondrial membrane potential as compared to control cells not subjected to the method.

In some embodiments, the cell is a non-human cell. In some embodiments, the cell is a eukaryotic cell.

In certain embodiments, the cell is a human cell. In some embodiments, the cell is an adult human cell. The cell may be from a human subject who is a young adult, middle aged or older. For example, the human subject may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

The cell may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

In certain embodiments, the cell may be from a human subject who is middle aged or older and has an ageing related condition such as wrinkles, sun damage, rough or dried skin, and/or sagging skin. In certain embodiments, the method of rejuvenating cells provided herein may improve skin condition, quality, and/or appearance in the human subject, such as, reduction in severity and/or number of wrinkles, improved skin tone, improved skin volume and elasticity, and the like.

In certain embodiments, the cell may be in a human subject who while in need for improving an ageing-related condition is otherwise healthy. In certain embodiments, the cell may be in a human subject who has disease such as a skin disease, e.g., psoriasis. In certain embodiments, the cell may be in a human subject who does not have psoriasis. In certain embodiments, the compositions and formulations disclosed herein for rejuvenating cells are not administered to a psoriatic lesion present on the skin of a human subject with psoriasis. In certain embodiments, the compositions and formulations disclosed herein for rejuvenating cells may be administered to an unbroken skin either by topical application or by superficial injection.

In certain embodiments, the method for rejuvenating skin cells in an adult human may include administering to the adult human a STAT3 inhibitor, where the adult human does not suffer from psoriasis. In certain embodiments, the method for rejuvenating skin cells in an adult human may include administering to the adult human a STAT3 inhibitor, where the adult human does not suffer from an autoimmune skin condition. Exemplary STAT3 inhibitors can include, but are not limited to: atovaquone, resveratrol, curcumin, pyrimethamine, celecoxib, napabucasin, niclosamide, cryptotanshinone, stattic, S31-201, H03867, SH-4-54, or Nifuroxazide.

In certain embodiments, the cell may be in a human subject who has a disease or condition which may be treated by decreasing the phenotypic, physiological, and/or genetic age of the cell or the tissue or organ in which the cell is present.

In certain embodiments, the cells may be differentiated cells, e.g., skin cells, lung cells (e.g., lung fibroblasts), liver cells (e.g., hepatocytes), muscle cells (e.g., cardiac muscle cells), pancreatic cells, immune cells, bone cells, brain cells (e.g., microglial cells, glial cells, astrocytes, neurons, etc.), eye cells (e.g., retinal cells, such as, retinal cell is a retinal ganglion cell, an amacrine cell, a horizontal cell, a bipolar cell, a photoreceptor cell, a Miller glial cell, a microglial cell, or a retinal pigmented epithelium cell), scalp cells (e.g., hair follicles), etc. In certain embodiments, the cells may be fibroblasts, e.g., skin fibroblasts, liver fibroblasts, lung fibroblasts, or skeletal muscle fibroblasts. In certain embodiments, the cells may be pancreatic islet cells. In certain embodiments, the cells may be T cells, B cells, macrophages, or dendritic cells. In certain embodiments, the cells may be bone marrow cells. In certain embodiments, the cells may be neural cells, glial cells, or astrocytes.

In certain embodiments, the TFs, compositions for increasing or decreasing the activity of a TF, and methods disclosed herein find use treating an adult human suffering from a condition or disease which can be treated by decreasing the age of the cells present in the adult. For example, by decreasing the age of skin cells, liver cells, lung cells, etc. For example, an adult human may be suffering from skin condition (e.g., autoimmune disease, vitiligo, dermatitis, etc.), a lung condition (e.g., inflammation and/or fibrosis), or a liver condition (e.g., liver fibrosis).

The composition, method, kit, or system described herein can include increasing activity of one or more of E2F Transcription Factor 3 (E2F3), Enhancer Of Zeste 2 Polycomb Repressive Complex 2 Subunit (EZH2), Homeobox protein DLX-6 (DLX6), Forkhead Box M1 (FOXM1), FOS Like 1, AP-1 Transcription Factor Subunit (FOSL1), Nuclear Factor Of Activated T Cells 4 (NFATC4), Myc proto-oncogene protein (MYC), Signal Transducer And Activator Of Transcription 4 (STAT4), GATA Binding Protein 3 (GATA3), Heat Shock Transcription Factor 2 (HSF2), Paired Box 4 (PAX4), NK2 Homeobox 2 (NKX2-2), SIM BHLH Transcription Factor 2 (S1M2), and Ventral Anterior Homeobox 1 (VAX1) in the cells of the tissue involved in the condition, e.g., cells in skin, cells in liver, cells in lung(s), etc. Treating the adults human suffering from a condition may include decreasing the activity of one or more of Signal Transducer And Activator Of Transcription 3 (STAT3), Zinc Finger Protein X-Linked (ZFX), Early growth response protein 1 (EGR1), Activating Transcription Factor 2 (ATF2), Vascular Endothelial Zinc Finger 1 (VEZF1), MYC Associated Zinc Finger Protein (MAZ), SRY-Box Transcription Factor 2 (SOX2), Paired Box 8 (PAX8), Zinc Finger Homeobox 3 (ZFHX3), Autophagy Related 4C Cysteine Peptidase (ATG4C), Autophagy Related 5 (ATG5), High Mobility Group Box 1 (HMGB1), GATA Binding Protein 2 (GATA2), and KLF Transcription Factor 4 (KLF4) in the cells of the tissue involved in the condition described herein, e.g., cells in skin, cells in liver, cells in lung(s), etc.

The composition, method, kit, or system described herein can include increasing the activity of one or more of E2F Transcription Factor 3 (E2F3), Enhancer Of Zeste 2 Polycomb Repressive Complex 2 Subunit (EZH2), Homeobox protein DLX-6 (DLX6), Forkhead Box M1 (FOXM1), FOS Like 1, AP-1 Transcription Factor Subunit (FOSL1), Nuclear Factor Of Activated T Cells 4 (NFATC4), Myc proto-oncogene protein (MYC), Signal Transducer And Activator Of Transcription 4 (STAT4), GATA Binding Protein 3 (GATA3), Heat Shock Transcription Factor 2 (HSF2), Paired Box 4 (PAX4), NK2 Homeobox 2 (NKX2-2), SIM BHLH Transcription Factor 2 (SIM2), and Ventral Anterior Homeobox 1 (VAX1) and decreasing the activity of one or more of Signal Transducer And Activator Of Transcription 3 (STAT3), Zinc Finger Protein X-Linked (ZFX), Early growth response protein 1 (EGR1), Activating Transcription Factor 2 (ATF2), Vascular Endothelial Zinc Finger 1 (VEZF1), MYC Associated Zinc Finger Protein (MAZ), SRY-Box Transcription Factor 2 (SOX2), Paired Box 8 (PAX8), Zinc Finger Homeobox 3 (ZFHX3), Autophagy Related 4C Cysteine Peptidase (ATG4C), Autophagy Related 5 (ATG5), High Mobility Group Box 1 (HMGB1), GATA Binding Protein 2 (GATA2), and KLF Transcription Factor 4 (KLF4) in the cells of the tissue involved in the condition, e.g., cells in skin, cells in liver, cells in lung(s), etc. For example, treating the adult human suffering from a condition described herein may include increasing activity of one or both of E2F3 and EZH2 and/or decreasing the activity of one or both of STAT3 and ZFX in the cells of the tissue involved in the condition, e.g., cells in skin, cells in liver, cells in lung(s), etc.

In certain embodiments, the method comprises increasing the activity of one or more of DLX6, E2F3, FOXM1, FOSL1, NFATC4, MYC, STAT4, GATA3, EZH2, HSF2, PAX4, NKX2-2, SIM2, or VAX1 transiently. In certain embodiments, the method comprises decreasing the activity of one or more of EGR1, ZFX, ATF4, VEZF1, MAZ, SOX2, PAX8, ZFHX3, ATG4C, ATG5, HMGB1, STAT3, GATA2, or KLF4 transiently. In some embodiments, the method comprises increasing the activity of one or more of DLX6, E2F3, FOXM1, FOSL1, NFATC4, MYC, STAT4, GATA3, EZH2, HSF2, PAX4, NKX2-2, SIM2, or VAX1 transiently and decreasing the activity of one or more of EGR1, ZFX, ATF4, VEZF1, MAZ, SOX2, PAX8, ZFHX3, ATG4C, ATG5, HMGB1, STAT3, GATA2, or KLF4. Transiently. For example, increasing the activity of E2F3 and/or EZH2 and/or decreasing the activity of STAT3 and/or ZFX for a period of time that is less than one month, less than three weeks, less than two weeks, less than one week, less than 5 days, less than 3 days, less than a day, less than 18 hours, less than 12 hours, less than 8 hours, less than 5 hours, or lesser, e.g., at least 1 hour.

In certain embodiments, the cell may not be from an adult human but may be from a child, toddler, or a baby who has a disease that gives the cells in the child, toddler, or a baby characteristics of an adult cell, such as, decreased cells division, increase in senescence, decrease in proteasome activity, increase in senescence-associated lysosomes, decrease in mitochondrial gene expression, decrease in metabolism gene expression, decrease in mitochondrial membrane potential, etc. as compared to the same cell from an age-matched child, toddler, or a baby. Thus, the disclosed methods may be used for treating a child, toddler, or a baby suffering from a disease which cause the cells of child, toddler, or a baby to have features of premature aging.

In certain embodiments, the method for rejuvenating an adult cell may include administering to an adult subject in need thereof a composition comprising DLX6, E2F3, FOXM1, FOSL1, NFATC4, MYC, STAT4, GATA3, EZH2, HSF2, PAX4, NKX2-2, SIM2, and/or VAX1, a polynucleic acid encoding DLX6, E2F3, FOXM1, FOSL1, NFATC4, MYC, STAT4, GATA3, EZH2, HSF2, PAX4, NKX2-2, SIM2, and/or VAX1, a polynucleic acid (e.g., in a CRISPR/Cas9 complex) for activating expression of DLX6, E2F3, FOXM1, FOSL1, NFATC4, MYC, STAT4, GATA3, EZH2, HSF2, PAX4, NKX2-2, SIM2, and/or VAX1, a polynucleic acid (e.g., in a CRISPR/Cas9 complex) for inhibiting expression of EGR1, ZFX, ATF4, VEZF1, MAZ, SOX2, PAX8, ZFHX3, ATG4C, ATG5, HMGB1, STAT3, GATA2, and/or KLF4. The route for delivery may be selected based on the disease. For example, an adult subject may have a skin condition (e.g., autoimmune disease, vitiligo, dermatitis, etc.), a lung condition (e.g., inflammation and/or fibrosis), or a liver condition (e.g., liver fibrosis).

The method provided herein can be performed in vivo, in situ, in vitro, ex vivo, or any combination thereof.

In some embodiments, the method provided herein does not comprise substantial changes of TERT-related gene expression or telomere length. Exemplary TERT-related genes include EREG, EEF1A2, ALDH1A1, and EPB41L3.

### Increasing Activity of a TF

Provided herein is a method comprising increasing the activity of one or more of DLX6, E2F3, FOXM1, FOSL1, NFATC4, MYC, STAT4, GATA3, EZH2, HSF2, PAX4, NKX2-2, SIM2, or VAX1 in a cell.

Increasing activity of a polypeptide, such as, one or more of DLX6, E2F3, FOXM1, FOSL1, NFATC4, MYC, STAT4, GATA3, EZH2, HSF2, PAX4, NKX2-2, SIM2, and/or VAX1 may encompass one or both of: increasing total level of the polypeptide in a cell and increasing the effect of the polypeptide. Level of a polypeptide in a cell may be increased by increasing expression of the polypeptide from an endogenous gene, introducing into the cell an exogeneous polynucleic acid encoding the polypeptide, introducing the polypeptide into the cells, increasing the stability of the polypeptide, etc. Effect of a polypeptide in the cell may be increased by expressing a mutated version of the polypeptide, introducing into the cell an activator of the polypeptide (e.g., a small molecule, a co-factor, or another transcriptional activator), fusing the polypeptide to a domain that increases its localization to the nucleus, etc. Increased effect of a TF may be assessed by assaying expression levels of one or more genes activated by the TFs. For example, increased effect of E2F3 and/or EZH2 may be assessed by assaying expression levels of one or more genes activated by these TFs. Increased level of a TF (e.g., E2F3 or EZH2) may be assessed by assaying expression levels of one or more genes activated by these TFs and/or measuring level of the TFs directly (e.g., by ELISA). In certain embodiments, the effect of a polypeptide may be increased in absence of an increase in level of the polypeptide. Unless specified otherwise, an increase or decrease in activity (e.g., effectiveness and/or level) of a polypeptide is in comparison to a control cell that has not been subjected to the increase or decrease in activity, respectively, of the polypeptide.

In certain embodiments, increasing the activity of one or more of DLX6, E2F3, FOXM1, FOSL1, NFATC4, MYC, STAT4, GATA3, EZH2, HSF2, PAX4, NKX2-2, SIM2, and/or VAX1 may encompass increasing the activity by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more as compared to a control cell.

In certain embodiments, the method comprises increasing the function of one or more of DLX6, E2F3, FOXM1, FOSL1, NFATC4, MYC, STAT4, GATA3, EZH2, HSF2, PAX4, NKX2-2, SIM2, and/or VAX1. Non-limiting examples of function can include mRNA level, protein level, mRNA level of any suitable downstream targets, protein level of any suitable downstream targets, level of transcription, level of translation, level of any suitable positive feedback loop, level of any suitable negative feedback loop, or activity of downstream signaling pathways.

In certain embodiments, the method comprises increasing the activity of DLX6, wherein increasing the activity of DLX6 comprises increasing the mRNA level of DLX6. In certain embodiments, the method comprises increasing the activity of E2F3, wherein increasing the activity of E2F3 comprises increasing the mRNA level of E2F3. In certain embodiments, the method comprises increasing the activity of FOXM1, wherein increasing the activity of FOXM1 comprises increasing the mRNA level of FOXM1. In certain embodiments, the method comprises increasing the activity of FOSL1, wherein increasing the activity of FOSL1 comprises increasing the mRNA level of FOSL1. In certain embodiments, the method comprises increasing the activity of NFATC4, wherein increasing the activity of NFATC4 comprises increasing the mRNA level of NFATC4. In certain embodiments, the method comprises increasing the activity of MYC, wherein increasing the activity of MYC comprises increasing the mRNA level of MYC. In certain embodiments, the method comprises increasing the activity of STAT4, wherein increasing the activity of STAT4 comprises increasing the mRNA level of STAT4. In certain embodiments, the method comprises increasing the activity of GATA3, wherein increasing the activity of GATA3 comprises increasing the mRNA level of GATA3. In certain embodiments, the method comprises increasing the activity of EZH2, wherein increasing the activity of EZH2 comprises increasing the mRNA level of EZH2. In certain embodiments, the method comprises increasing the activity of HSF2, wherein increasing the activity of HSF2 comprises increasing the mRNA level of HSF2. In certain embodiments, the method comprises increasing the activity of PAX4, wherein increasing the activity of PAX4 comprises increasing the mRNA level of PAX4. In certain embodiments, the method comprises increasing the activity of NKX2-2, wherein increasing the activity of NKX2-2 comprises increasing the mRNA level of NKX2-2. In certain embodiments, the method comprises increasing the activity of SIM2, wherein increasing the activity of SIM2 comprises increasing the level of mRNA SIM2. In certain embodiments, the method comprises increasing the activity of VAX1, wherein increasing the activity of VAX1 comprises increasing the mRNA level of VAX1. In certain embodiments, the method comprises increasing the activity of DLX6, wherein increasing the activity of DLX6 comprises increasing the protein level of DLX6. In certain embodiments, the method comprises increasing the activity of E2F3, wherein increasing the activity of E2F3 comprises increasing the protein level of E2F3. In certain embodiments, the method comprises increasing the activity of FOXM1, wherein increasing the activity of FOXM1 comprises increasing the protein level of FOXM1. In certain embodiments, the method comprises increasing the activity of FOSL1, wherein increasing the activity of FOSL1 comprises increasing the protein level of FOSL1. In certain embodiments, the method comprises increasing the activity of NFATC4, wherein increasing the activity of NFATC4 comprises increasing the protein level of NFATC4. In certain embodiments, the method comprises increasing the activity of MYC, wherein increasing the activity of MYC comprises increasing the protein level of MYC. In certain embodiments, the method comprises increasing the activity of STAT4, wherein increasing the activity of STAT4 comprises increasing the protein level of STAT4. In certain embodiments, the method comprises increasing the activity of GATA3, wherein increasing the activity of GATA3 comprises increasing the protein level of GATA3. In certain embodiments, the method comprises increasing the activity of EZH2, wherein increasing the activity of EZH2 comprises increasing the protein level of EZH2. In certain embodiments, the method comprises increasing the activity of HSF2, wherein increasing the activity of HSF2 comprises increasing the protein level of HSF2. In certain embodiments, the method comprises increasing the activity of PAX4, wherein increasing the activity of PAX4 comprises increasing the protein level of PAX4. In certain embodiments, the method comprises increasing the activity of NKX2-2, wherein increasing the activity of NKX2-2 comprises increasing the protein level of NKX2-2. In certain embodiments, the method comprises increasing the activity of SIM2, wherein increasing the activity of SIM2 comprises increasing the level of protein SIM2. In certain embodiments, the method comprises increasing the activity of VAX1, wherein increasing the activity of VAX1 comprises increasing the protein level of VAX1.

In certain embodiments, increasing the activity of DLX6 may encompass increasing the activity of DLX6 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of DLX6 may encompass increasing the mRNA level of DLX6 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of DLX6 may encompass increasing the protein level of DLX6 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of E2F3 may encompass increasing the activity of E2F3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of E2F3 may encompass increasing the mRNA level of E2F3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of E2F3 may encompass increasing the protein level of E2F3 in a rejuvenated cell by at least 1%. 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of FOXM1 may encompass increasing the activity of FOXM1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of FOXM1 may encompass increasing the mRNA level of FOXM1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of FOXM1 may encompass increasing the protein level of FOXM1 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of FOXL1 may encompass increasing the activity of FOXL1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of FOXL1 may encompass increasing the mRNA level of FOXL1 in a rejuvenated cell by at least 1%, 2%, 3%. 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of FOXL1 may encompass increasing the protein level of FOXL1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of NFATC4 may encompass increasing the activity of NFATC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of NFATC4 may encompass increasing the mRNA level of NFATC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of NFATC4 may encompass increasing the protein level of NFATC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of MYC may encompass increasing the activity of MYC in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of MYC may encompass increasing the mRNA level of MYC in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of MYC may encompass increasing the protein level of MYC in a rejuvenated cell by 1%, 2%, 3%, 4%, 5% 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of STAT4 may encompass increasing the activity of STAT4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of STAT4 may encompass increasing the mRNA level of STAT4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of STAT4 may encompass increasing the protein level of STAT4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of GATA3 may encompass increasing the activity of GATA3 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of GATA3 may encompass increasing the mRNA level of GATA3 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of GATA3 may encompass increasing the protein level of GATA3 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of EZH2 may encompass increasing the activity of EZH2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of EZH2 may encompass increasing the mRNA level of EZH2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of EZH2 may encompass increasing the protein level of EZH2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of HSF2 may encompass increasing the activity of HSF2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of HSF2 may encompass increasing the mRNA level of HSF2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of HSF2 may encompass increasing the protein level of HSF2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of PAX4 may encompass increasing the activity of PAX4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of PAX4 may encompass increasing the mRNA level of PAX4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of PAX4 may encompass increasing the protein level of PAX4 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of NKX2-2 may encompass increasing the activity of NKX2-2 in a rejuvenated cell by 1%, 2%, 3%. 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of NKX2-2 may encompass increasing the mRNA level of NKX2-2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of NKX2-2 may encompass increasing the protein level of NKX2-2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

In certain embodiments, increasing the activity of SIM2 may encompass increasing the activity of SIM2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of SIM2 may encompass increasing the mRNA level of SIM2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, increasing the activity of SIM2 may encompass increasing the protein level of SIM2 in a rejuvenated cell by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

Level of gene expression in a cell can be measured by any suitable means, such as qPCR, sanger sequencing, RNA sequencing, bulk RNA sequencing, single cell RNA sequencing, RT PCR, northern blot, or microarray. Level of protein expression in a cell can be measured by any suitable means, such as immunofluorescent imaging, immunohistochemistiy, flow cytometry, or western blotting. Level of a protein in a cell can be increased by any suitable means, such as, by increasing transcription of the endogenous gene encoding the protein, by introducing a polynucleic acid encoding the protein into the cells, by introducing the protein into the cells, and the like. In some embodiments, rejuvenation can be determined by epigenetic alternation of the TFs described herein. Epigenetic alternation can include increase in methylation, decrease in methylation, increase in acetylation, or decrease in acetylation. For example, an rejuvenated cell can have increased level of histone 3 lysine 9 trimethylation (H3K9me3). Epigenetic landscape can be determined using ATACseq, bisulfite (BS) sequencing, CUT&Tag, ChIP-Seq, western blotting, immunofluorescence imaging, immunohistochemistry.

In certain embodiments, increasing the level of E2F3 may include increasing the expression level of E2F3 by, e.g., increasing transcription of the endogenous E2F3 gene encoding the E2F3 protein. In certain embodiments, increasing the expression level of EZH2 may include increasing transcription of the EZH2 gene encoding the EZH2 protein. In certain embodiments, increasing the level of DLX6 may include increasing the expression level of DLX6 by, e.g., increasing transcription of the endogenous DLX6 gene encoding the DLX6 protein. In certain embodiments, increasing the expression level of DLX6 may include increasing transcription of the DLX6 gene encoding the DLX6 protein. In certain embodiments, increasing the level of FOXM1 may include increasing the expression level of FOXM1 by, e.g., increasing transcription of the endogenous FOXM1 gene encoding the FOXM1 protein. In certain embodiments, increasing the expression level of FOXM1 may include increasing transcription of the FOXM1 gene encoding the FOXM1 protein. In certain embodiments, increasing the level of FOSL1 may include increasing the expression level of FOSL1 by, e.g., increasing transcription of the endogenous FOSL1 gene encoding the FOSL1 protein. In certain embodiments, increasing the expression level of FOSL1 may include increasing transcription of the FOSL1 gene encoding the FOSL1 protein. In certain embodiments, increasing the level of NFATC4 may include increasing the expression level of NFATC4 by, e.g., increasing transcription of the endogenous NFATC4 gene encoding the NFATC4 protein. In certain embodiments, increasing the expression level of NFATC4 may include increasing transcription of the NFATC4 gene encoding the NFATC4 protein. In certain embodiments, increasing the level of MYC may include increasing the expression level of MYC by, e.g., increasing transcription of the endogenous MYC gene encoding the MYC protein. In certain embodiments, increasing the expression level of MYC may include increasing transcription of the MYC gene encoding the MYC protein. In certain embodiments, increasing the level of STAT4 may include increasing the expression level of STAT4 by, e.g., increasing transcription of the endogenous STAT4 gene encoding the STAT4 protein. In certain embodiments, increasing the expression level of STAT4 may include increasing transcription of the STAT4 gene encoding the STAT4 protein. In certain embodiments, increasing the level of GATA3 may include increasing the expression level of GATA3 by, e.g., increasing transcription of the endogenous GATA3 gene encoding the GATA3 protein. In certain embodiments, increasing the expression level of GATA3 may include increasing transcription of the GATA3 gene encoding the GATA3 protein. In certain embodiments, increasing the level of EZH2 may include increasing the expression level of EZH2 by, e.g., increasing transcription of the endogenous EZH2 gene encoding the EZH2 protein. In certain embodiments, increasing the expression level of EZH2 may include increasing transcription of the EZH2 gene encoding the EZH2 protein. In certain embodiments, increasing the level of HSF2 may include increasing the expression level of HSF2 by, e.g., increasing transcription of the endogenous HSF2 gene encoding the HSF2 protein. In certain embodiments, increasing the expression level of HSF2 may include increasing transcription of the HSF2 gene encoding the HSF2 protein. In certain embodiments, increasing the level of PAX4 may include increasing the expression level of PAX4 by, e.g., increasing transcription of the endogenous PAX4 gene encoding the PAX4 protein. In certain embodiments, increasing the expression level of PAX4 may include increasing transcription of the PAX4 gene encoding the PAX4 protein. In certain embodiments, increasing the level of NKX2-2 may include increasing the expression level of NKX2-2 by, e.g., increasing transcription of the endogenous NKX2-2 gene encoding the DLX6 protein. In certain embodiments, increasing the expression level of NKX2 may include increasing transcription of the NKX2-2 gene encoding the NKX2-2 protein. In certain embodiments, increasing the level of SIM2 may include increasing the expression level of SIM2 by, e.g., increasing transcription of the endogenous SIM2 gene encoding the SIM2 protein. In certain embodiments, increasing the expression level of SIM2 may include increasing transcription of the SIM2 gene encoding the SIM2 protein. In certain embodiments, increasing the level of VAX1 may include increasing the expression level of VAX1 by, e.g., increasing transcription of the endogenous VAX1 gene encoding the VAX1 protein. In certain embodiments, increasing the expression level of VAX1 may include increasing transcription of the VAX1 gene encoding the VAX1 protein.

In certain embodiments, increasing transcription of the gene encoding the protein may include introducing into the cell a CRISPR/guide RNA that activates expression of the protein. In certain embodiments, the guide RNA may bind to a region in the promoter of the gene encoding the protein. In certain embodiments, a guide RNA that binds to a sequence in a regulatory region of the E2F3 gene may include the sequences shown in Table 1.

In certain embodiments, a guide RNA that binds to a sequence in a regulatory region of the EZH2 gene may include the sequences shown in Table 1.

TABLE 1
Sample Guide RNA Sequences for CRISPR-
Mediated Gene Activation

| SEQ ID NO: | NAME | SEQUENCE |
|---|---|---|
| 1 | E2F3 Guide 1 | GGGGCTGTGGGAACGGTCCT |
| 2 | E2F3 Guide 2 | GGCCCAATGGGCTGTGGGAA |
| 3 | EZH2 Guide 1 | GCCCACGCAGAGTGCGCTCA |
| 4 | EZH2 Guide 2 | GCGGGCGCCCGTCCAATCAC |

In certain embodiments, the Cas protein may be conjugated to a transcriptional activator. In certain embodiments, the transcriptional activator may be VP16, VP64, p65, p300 catalytic domain, TET1 catalytic domain, TDG, Ldbl self-associated domain, SAM activator (VP64, p65, HSF1), or VPR (VP64, p65, Rta).

In certain embodiments, an RNA-guided transcriptional activator of E2F3, DLX6, FOXM1, FOSL1, NFATC4, MYC, STAT4, GATA3, EZH2, HSF2, PAX4, NKX2-2, SIM2, and/or VAX1 gene may be introduced in the cells as a guide RNA and transcriptional activator or as one or more polynucleic acids encoding the guide RNA and transcriptional activator.

In certain embodiments, increasing the level of E2F3 in the cells may include introducing a polynucleic acid encoding the E2F3 into the cells, where the cell expresses the E2F3 encoded by the polynucleic acid. In certain embodiments, increasing the level of DLX6 in the cells may include introducing a polynucleic acid encoding the DLX6 into the cells, where the cell expresses the DLX6 encoded by the polynucleic acid. In certain embodiments, increasing the level of FOXM1 in the cells may include introducing a polynucleic acid encoding the FOXM1 into the cells, where the cell expresses the FOXM1 encoded by the polynucleic acid. In certain embodiments, increasing the level of FOSL1 in the cells may include introducing a polynucleic acid encoding the FOSL1 into the cells, where the cell expresses the FOSL1 encoded by the polynucleic acid. In certain embodiments, increasing the level of NFATC4 in the cells may include introducing a polynucleic acid encoding the NFATC4 into the cells, where the cell expresses the NFATC4 encoded by the polynucleic acid. In certain embodiments, increasing the level of MYC in the cells may include introducing a polynucleic acid encoding the MYC into the cells, where the cell expresses the MYC encoded by the polynucleic acid. In certain embodiments, increasing the level of STAT4 in the cells may include introducing a polynucleic acid encoding the STAT4 into the cells, where the cell expresses the STAT4 encoded by the polynucleic acid. In certain embodiments, increasing the level of GATA3 in the cells may include introducing a polynucleic acid encoding the GATA3 into the cells, where the cell expresses the GATA3 encoded by the polynucleic acid. In certain embodiments, increasing the level of EZH2 in the cells may include introducing a polynucleic acid encoding the EZH2 into the cells, where the cell expresses the EZH2 encoded by the polynucleic acid. In certain embodiments, increasing the level of HSF2 in the cells may include introducing a polynucleic acid encoding the HSF2 into the cells, where the cell expresses the HSF2 encoded by the polynucleic acid. In certain embodiments, increasing the level of PAX4 in the cells may include introducing a polynucleic acid encoding the PAX4 into the cells, where the cell expresses the PAX4 encoded by the polynucleic acid. In certain embodiments, increasing the level of NKX2-2 in the cells may include introducing a polynucleic acid encoding the NKX2-2 into the cells, where the cell expresses the NKX2-2 encoded by the polynucleic acid. In certain embodiments, increasing the level of SIM2 in the cells may include introducing a polynucleic acid encoding the SIM2 into the cells, where the cell expresses the SIM2 encoded by the polynucleic acid. In certain embodiments, increasing the level of VAX1 in the cells may include introducing a polynucleic acid encoding the VAX1 into the cells, where the cell expresses the VAX1 encoded by the polynucleic acid. The polynucleic acid may be DNA (e.g., plasmid, viral vector, or cDNA) or RNA.

In certain embodiments, increasing the expression level of E2F3 in the cells may include introducing exogenous E2F3 into the cells. In certain embodiments, increasing the expression level of DLX6 in the cells may include introducing exogenous DLX6 into the cells. In certain embodiments, increasing the expression level of FOXM1 in the cells may include introducing exogenous FOXM1 into the cells. In certain embodiments, increasing the expression level of FOSL1 in the cells may include introducing exogenous FOSL1 into the cells. In certain embodiments, increasing the expression level of NFATC4 in the cells may include introducing exogenous NFATC4 into the cells. In certain embodiments, increasing the expression level of NFATC4 in the cells may include introducing exogenous NFATC4 into the cells. In certain embodiments, increasing the expression level of MYC in the cells may include introducing exogenous MYC into the cells. In certain embodiments, increasing the expression level of STAT4 in the cells may include introducing exogenous STAT4 into the cells. In certain embodiments, increasing the expression level of GATA3 in the cells may include introducing exogenous GATA3 into the cells. In certain embodiments, increasing the expression level of EZH2 in the cells may include introducing exogenous EZH2 into the cells. In certain embodiments, increasing the expression level of HSF2 in the cells may include introducing exogenous HSF2 into the cells. In certain embodiments, increasing the expression level of PAX4 in the cells may include introducing exogenous PAX4 into the cells. In certain embodiments, increasing the expression level of NKX2-2 in the cells may include introducing exogenous NKX2-2 into the cells. In certain embodiments, increasing the expression level of SIM2 in the cells may include introducing exogenous SIM2 into the cells. In certain embodiments, increasing the expression level of VAX1 in the cells may include introducing exogenous VAX1 into the cells.

In certain embodiments, increasing the activity of E2F3 may include contacting the cells with an activator of E2F3, wherein the activator increases binding of E2F3 to an expression control element of a target gene expressed under the control of E2F3. In certain embodiments, increasing the activity of DLX6 may include contacting the cells with an activator of DLX6, wherein the activator increases binding of DLX6 to an expression control element of a target gene expressed under the control of DLX6. In certain embodiments, increasing the activity of FOXM1 may include contacting the cells with an activator of FOXM1, wherein the activator increases binding of FOXM1 to an expression control element of a target gene expressed under the control of FOXM1. In certain embodiments, increasing the activity of FOSL1 may include contacting the cells with an activator of FOSL1, wherein the activator increases binding of FOSL1 to an expression control element of a target gene expressed under the control of FOSL1. In certain embodiments, increasing the activity of NFATC4 may include contacting the cells with an activator of NFATC4, wherein the activator increases binding of NFATC4 to an expression control element of a target gene expressed under the control of NFATC4. In certain embodiments, increasing the activity of MYC may include contacting the cells with an activator of MYC, wherein the activator increases binding of MYC to an expression control element of a target gene expressed under the control of MYC. In certain embodiments, increasing the activity of STAT4 may include contacting the cells with an activator of STAT4, wherein the activator increases binding of STAT4 to an expression control element of a target gene expressed under the control of STAT4. In certain embodiments, increasing the activity of EZH2 may include contacting the cells with an activator of EZH2, wherein the activator increases binding of EZH2 to an expression control element of a target gene expressed under the control of EZH2. In certain embodiments, increasing the activity of HSF2 may include contacting the cells with an activator of HSF2, wherein the activator increases binding of HSF2 to an expression control element of a target gene expressed under the control of HSF2. In certain embodiments, increasing the activity of PAX4 may include contacting the cells with an activator of PAX4, wherein the activator increases binding of PAX4 to an expression control element of a target gene expressed under the control of PAX4. In certain embodiments, increasing the activity of NKX2-2 may include contacting the cells with an activator of NKX2-2, wherein the activator increases binding of NKX2-2 to an expression control element of a target gene expressed under the control of NKX2-2. In certain embodiments, increasing the activity of SIM2 may include contacting the cells with an activator of SIM2, wherein the activator increases binding of SIM2 to an expression control element of a target gene expressed under the control of SIM2. In certain embodiments, increasing the activity of VAX1 may include contacting the cells with an activator of VAX1, wherein the activator increases binding of VAX1 to an expression control element of a target gene expressed under the control of VAX1.

### Decreasing Activity of a TF

Provided herein is a method comprises decreasing the activity of EGR1, ZFX, ATF4, VEZF1, MAZ, SOX2, PAX8, ZFHX3, ATG4C, ATG5, HMGB1, STAT3, GATA2, and/or KLF4 in a cell.

Decreasing the activity of a polypeptide such as EGR1, ZFX, ATF4, VEZF1, MAZ, SOX2, PAX8, ZFHX3, ATG4C, ATG5, HMGB1, STAT3, GATA2, and/or KLF4 may encompass one or both of: decreasing total level of the polypeptide in a cell and decreasing the effect of the polypeptide. Level of a polypeptide in a cell may be decreased by decreasing expression of the polypeptide from an endogenous gene, decreasing the level of the mRNA encoding the polypeptide, inhibiting translation of the mRNA, etc. Effect of a polypeptide in the cell may be decreased by expressing a mutated version of the polypeptide, introducing into the cell an inhibitor of the polypeptide (e.g., a small molecule), fusing the polypeptide to a domain that decreases its localization to the nucleus, fusing the polypeptide to a domain that decreases its stability, etc. Decreased effect of a TF may be assessed by assaying expression levels of one or more genes activated by the TFs.

In certain embodiments, decreasing the activity of one or more of EGR1, ZFX, ATF4, VEZF1, MAZ, SOX2, PAX8, ZFHX3, ATG4C, ATG5, HMGB1, STAT3, GATA2, and/or KLF4 may encompass decreasing the activity by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more as compared to a control cell.

In certain embodiments, the method comprises decreasing the function of one or more of EGR1, ZFX, ATF4, VEZF1, MAZ, SOX2, PAX8, ZFHX3, ATG4C, ATG5, HMGB1, STAT3, GATA2, and/or KLF4. Non-limiting examples of function can include mRNA level, protein level, mRNA level of any suitable downstream targets, protein level of any suitable downstream targets, level of transcription, level of translation, level of any suitable positive feedback loop, level of any suitable negative feedback loop, or activity of downstream signaling pathways.

In certain embodiments, the method comprises decreasing the activity of EGR1, wherein decreasing the activity of EGR1 comprises decreasing the mRNA level of EGR1. In certain embodiments, the method comprises decreasing the activity of ZFX, wherein decreasing the activity of ZFX comprises decreasing the mRNA level of ZFX. In certain embodiments, the method comprises decreasing the activity of ATF4, wherein decreasing the activity of ATF4 comprises decreasing the mRNA level of ATF4. In certain embodiments, the method comprises decreasing the activity of VEZF1, wherein decreasing the activity of VEZF1 comprises decreasing the mRNA level of VEZF1. In certain embodiments, the method comprises decreasing the activity of MAZ, wherein decreasing the activity of MAZ comprises decreasing the mRNA level of MAZ. In certain embodiments, the method comprises decreasing the activity of SOX2, wherein decreasing the activity of SOX2 comprises decreasing the mRNA level of SOX2. In certain embodiments, the method comprises decreasing the activity of PAX8, wherein decreasing the activity of PAX8 comprises decreasing the mRNA level of PAX8. In certain embodiments, the method comprises decreasing the activity of ZFHX3, wherein decreasing the activity of ZFHX3 comprises decreasing the mRNA level of ZFHX3. In certain embodiments, the method comprises decreasing the activity of ATG4C, wherein decreasing the activity of ATG4C comprises decreasing the mRNA level of ATG4C. In certain embodiments, the method comprises decreasing the activity of ATG5, wherein decreasing the activity of ATG5 comprises decreasing the mRNA level of ATG5. In certain embodiments, the method comprises decreasing the activity of HMGB1, wherein decreasing the activity of HMGB1 comprises decreasing the mRNA level of HMGB1. In certain embodiments, the method comprises decreasing the activity of STAT3, wherein decreasing the activity of STAT3 comprises decreasing the mRNA level of STAT3. In certain embodiments, the method comprises decreasing the activity of GATA2, wherein decreasing the activity of GATA2 comprises decreasing the mRNA level of GATA2. In certain embodiments, the method comprises decreasing the activity of KLF4, wherein decreasing the activity of KLF4 comprises decreasing the mRNA level of KLF4. In certain embodiments, the method comprises decreasing the activity of EGR1, wherein decreasing the activity of EGR1 comprises decreasing the protein level of EGR1. In certain embodiments, the method comprises decreasing the activity of ZFX, wherein decreasing the activity of ZFX comprises decreasing the protein level of ZFX. In certain embodiments, the method comprises decreasing the activity of ATF4, wherein decreasing the activity of ATF4 comprises decreasing the protein level of ATF4. In certain embodiments, the method comprises decreasing the activity of VEZF1, wherein decreasing the activity of VEZF1 comprises decreasing the protein level of VEZF1. In certain embodiments, the method comprises decreasing the activity of MAZ, wherein decreasing the activity of MAZ comprises decreasing the protein level of MAZ. In certain embodiments, the method comprises decreasing the activity of SOX2, wherein decreasing the activity of SOX2 comprises decreasing the protein level of SOX2. In certain embodiments, the method comprises decreasing the activity of PAX8, wherein decreasing the activity of PAX8 comprises decreasing the protein level of PAX8. In certain embodiments, the method comprises decreasing the activity of ZFHX3, wherein decreasing the activity of ZFHX3 comprises decreasing the protein level of ZFHX3. In certain embodiments, the method comprises decreasing the activity of ATG4C, wherein decreasing the activity of ATG4C comprises decreasing the protein level of ATG4C. In certain embodiments, the method comprises decreasing the activity of ATG5, wherein decreasing the activity of ATG5 comprises decreasing the protein level of ATG5. In certain embodiments, the method comprises decreasing the activity of HMGB1, wherein decreasing the activity of HMGB1 comprises decreasing the protein level of HMGB1. In certain embodiments, the method comprises decreasing the activity of STAT3, wherein decreasing the activity of STAT3 comprises decreasing the protein level of STAT3. In certain embodiments, the method comprises decreasing the activity of GATA2, wherein decreasing the activity of GATA2 comprises decreasing the protein level of GATA2. In certain embodiments, the method comprises decreasing the activity of KLF4, wherein decreasing the activity of KLF4 comprises decreasing the protein level of KLF4.

In certain embodiments, decreasing the activity of EGR1 may encompass decreasing the activity of EGR1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of EGR1 may encompass decreasing the mRNA level of EGR1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of EGR1 may encompass decreasing the protein level of EGR1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFX may encompass decreasing the activity of ZFX in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFX may encompass decreasing the mRNA level of ZFX in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFX may encompass decreasing the protein level of ZFX in a rejuvenated cell by at least 1%, 2%, 3%. 4%, 5%, 6%. 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATF4 may encompass decreasing the activity of ATF4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATF4 may encompass decreasing the mRNA level of ATF4 in a rejuvenated cell by at least 1%, 2%, 3%, 4%, 5%. 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATF4 may encompass decreasing the protein level of ATF4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of VEZF1 may encompass decreasing the activity of VEZF1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of VEZF1 may encompass decreasing the mRNA level of VEZF1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of VEZF1 may encompass decreasing the protein level of VEZF1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of MAZ may encompass decreasing the activity of MAZ in a rejuvenated cell by at least 1%, 2%, 3%, 4%, 5%, 6%, 7%. 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of MAZ may encompass decreasing the mRNA level of MAZ in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of MAZ may encompass decreasing the protein level of MAZ in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of SOX2 may encompass decreasing the activity of SOX2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of SOX2 may encompass decreasing the mRNA level of SOX2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of SOX2 may encompass decreasing the protein level of SOX2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of PAX8 may encompass decreasing the activity of PAX8 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of PAX8 may encompass decreasing the mRNA level of PAX8 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of PAX8 may encompass decreasing the protein level of PAX8 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFHX3 may encompass decreasing the activity of ZFHX3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFHX3 may encompass decreasing the mRNA level of ZFHX3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ZFHX3 may encompass decreasing the protein level of ZFHX3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATGC4 may encompass decreasing the activity of ATGC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATGC4 may encompass decreasing the mRNA level of ATGC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATGC4 may encompass decreasing the protein level of ATGC4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATG5 may encompass decreasing the activity of ATG5 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATG5 may encompass decreasing the mRNA level of ATG5 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of ATG5 may encompass decreasing the protein level of ATG5 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of HMGB1 may encompass decreasing the activity of HMGB1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of HMGB1 may encompass decreasing the mRNA level of HMGB1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of HMGB1 may encompass decreasing the protein level of HMGB1 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of STAT3 may encompass decreasing the activity of STAT3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of STAT3 may encompass decreasing the mRNA level of STAT3 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of STAT3 may encompass decreasing the protein level of STAT3 in a rejuvenated cell by at least 1%, 2%, 3%, 4%, 5%, 6%. 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of GATA2 may encompass decreasing the activity of GATA2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of GATA2 may encompass decreasing the mRNA level of GATA2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of GATA2 may encompass decreasing the protein level of GATA2 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of KLF4 may encompass decreasing the activity of KLF4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of KLF4 may encompass decreasing the mRNA level of KLF4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell. In certain embodiments, decreasing the activity of KLF4 may encompass decreasing the protein level of KLF4 in a rejuvenated cell by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100% or more as compared to a control un-rejuvenated cell.

Level of a protein in a cell can be decreased by any suitable means, such as, by decreasing transcription of the endogenous gene encoding the protein, by introducing a polynucleic acid into the cells that specifically degrades mRNA encoding the protein, by introducing into the cells a polynucleic acid that interferes with translation of the mRNA encoding the protein, and the like.

In certain embodiments, decreasing the level of EGR1 may include decreasing the expression level EGR1 by, e.g., decreasing transcription of the endogenous EGR1 gene encoding the EGR1 protein. In certain embodiments, decreasing the level of ZFX may include decreasing the expression level ZFX by, e.g., decreasing transcription of the endogenous ZFX gene encoding the ZFX protein. In certain embodiments, decreasing the level of ATF4 may include decreasing the expression level ATF4 by, e.g., decreasing transcription of the endogenous ATF4 gene encoding the ATF4 protein. In certain embodiments, decreasing the level of VEZF1 may include decreasing the expression level VEZF1 by, e.g., decreasing transcription of the endogenous VEZF1 gene encoding the VEZF1 protein. In certain embodiments, decreasing the level of MAZ may include decreasing the expression level MAZ by, e.g., decreasing transcription of the endogenous MAZ gene encoding the MAZ protein. In certain embodiments, decreasing the level of SOX2 may include decreasing the expression level SOX2 by, e.g., decreasing transcription of the endogenous SOX2 gene encoding the SOX2 protein. In certain embodiments, decreasing the level of PAX8 may include decreasing the expression level PAX8 by, e.g., decreasing transcription of the endogenous PAX8 gene encoding the PAX8 protein. In certain embodiments, decreasing the level of ZFHX3 may include decreasing the expression level ZFHX3 by, e.g., decreasing transcription of the endogenous ZF1X3 gene encoding the ZFHX3 protein. In certain embodiments, decreasing the level of ATG4C may include decreasing the expression level ATG4C by, e.g., decreasing transcription of the endogenous ATG4C gene encoding the ATG4C protein. In certain embodiments, decreasing the level of ATG5 may include decreasing the expression level ATG5 by, e.g., decreasing transcription of the endogenous ATG5 gene encoding the ATG5 protein. In certain embodiments, decreasing the level of HMGB1 may include decreasing the expression level HMGB1 by, e.g., decreasing transcription of the endogenous HMGB1 gene encoding the HMGB1 protein. In certain embodiments, decreasing the level of STAT3 may include decreasing the expression level STAT3 by, e.g., decreasing transcription of the endogenous STAT3 gene encoding the STAT3 protein. In certain embodiments, decreasing the level of GATA2 may include decreasing the expression level GATA2 by, e.g., decreasing transcription of the endogenous GATA2 gene encoding the GATA2 protein. In certain embodiments, decreasing the level of KLF4 may include decreasing the expression level KLF4 by, e.g., decreasing transcription of the endogenous KLF4 gene encoding the KLF4 protein.

In certain embodiments, decreasing transcription of the gene encoding the protein may include introducing into the cell a Cas/guide RNA that suppresses expression of the protein. In certain embodiments, the guide RNA may bind to a region in the promoter of the gene encoding the protein or in the coding region. In certain embodiments, a guide RNA that binds to a sequence in the STAT3 gene may include the sequences found in Table 2.

In certain embodiments, a guide RNA that binds to a sequence in the ZFX gene may include the sequences found in Table 2.

TABLE 2
Sample Guide RNA Sequences for CRISPR-
Mediated Gene Suppression

| SEQ ID NO: | NAME | SEQUENCE |
|---|---|---|
| 5 | STAT3 Guide 1 | GCCAGGGAGCAGGAAATCGG |
| 6 | STAT3 Guide 2 | GGGATCCTGGACAGGCACCC |
| 7 | ZFX Guide 1 | GGACTCACCGGACGGACGTG |
| 8 | ZFX Guide 2 | GGGCGGCACCCGGGACTCAC |

In certain embodiments, the Cas protein may be conjugated to a transcriptional repressor. In certain embodiments, the transcriptional repressor may be KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP.

In certain embodiments, decreasing level of STAT3 and/or ZFX may include contacting the cells with one or more antisense oligonucleotides. By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can lead to degradation of the target RNA or inhibit its translation or both. Such molecules include, for example, antisense RNA or DNA molecules, interference RNA (RNAi), micro RNA (miRNA), decoy RNA molecules, siRNA, that hybridize to at least a portion of the target polynucleic acid. As such, these compounds may be introduced in the form of single-stranded, double-stranded, partially single-stranded, or circular oligomeric compounds.

In certain embodiments, the method comprises decreasing the activity of STAT3 and/or ZFX by administering an inhibitor, such as, antibody or a small molecule that inhibits protein activity. Exemplary STAT3 inhibitors can include, but are not limited to: atovaquone, resveratrol, curcumin, pyrimethamine, celecoxib, napabucasin, niclosamide, cryptotanshinone, stattic, S31-201, HO3867, SH-4-54, or Nifuroxazide.

In certain embodiments, the method comprises decreasing the activity of STAT3 by administering an inhibitor of STAT3 that is a STAT3 decoy molecule having a consensus sequence of the STAT3 binding. Exemplary, STAT3 decoy oligodeoxynucleotide or cyclic STAT3 decoy oligodeoxynucleotide are described in U.S. Pat. No. 8,722,640.

In certain embodiments, an RNA-guided transcriptional repressor of EGR1, ZFX, ATF4, VEZF1, MAZ, SOX2, PAX8, ZFHX3, ATG4C, ATG5, HMGB1, STAT3, GATA2, and/or KLF4 may be introduced in the cells as a guide RNA and transcriptional repressor or as one or more polynucleic acids encoding the guide RNA and transcriptional repressor.

### Methods of Production

In some embodiments, the polypeptides for introducing into a cell to increase activity of the polypeptide in the cell may be produced using any suitable method including recombinant and non-recombinant methods (e.g., chemical synthesis).

A composition disclosed herein can be produced by various methods in any quantity. For example, a composition disclosed herein can be produced in an amount of about 0.5 microgram, 1 microgram, about 1 milligram, about 1 gram, about 1 kilogram, or more. Non-limiting examples of production methods include in vitro transcription methods, polymerase chain transcription (PCT), recombinant overexpression (e.g., in E. coli, R. sulfidophilum, or other in vitro systems), transfer RNA (tRNA) scaffold methods, enzymatic methods, chemical methods, solid-phase oligonucleotide synthesis, solid-phase chemical synthesis, ribozyme cleavage methods, T4 ligation methods, position-selective labeling of RNA (PLOR), T7 RNA polymerase in vitro methods, T3 RNA polymerase in vitro methods, SP6 RNA polymerase in vitro methods, phosphoramidite chemistry, cell-free polynucleic acid expression methods, or a combination thereof. Non-limiting examples of purification methods include precipitation and solvent extraction, ultracentrifugation, polyacrylamide gel electrophoresis (PAGE), liquid chromatography (e.g., reversed-phase ion-pairing HPLC (RP-IP-HPLC), ion-exchange HPLC (IE-HPLC), ion-exchange fast-performance liquid chromatography (IE-FPLC), affinity chromatography (e.g., systematic evolution of ligands by exponential enrichment (SELEX), and size-exclusion chromatography (SEC)), or a combination thereof. Purification methods can be used to achieve varying degrees of purity of a composition disclosed herein, e.g., at least 80% purity, at least 85% purity, at least 90% purity, at least 91% purity, at least 92% purity, at least 93% purity, at least 94% purity, at least 95% purity, at least 96% purity, at least 97% purity, at least 98% purity, at least 99% purity, or at least 99.99%. Methods for the preparation of compositions comprising the compositions described herein include formulating the compositions with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a composition is dissolved, emulsions comprising a composition, or a solution containing liposomes, micelles, nanoparticles, vesicles, microvesicles, or nanovesicles comprising a composition as disclosed herein. Semi-solid compositions include, for example, gels, suspensions, lotions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

A polynucleotide described herein can be assembled by a variety of methods, e.g., by automated solid-phase synthesis. A polynucleotide can be constructed using standard solid-phase DNA/RNA synthesis. A polynucleotide can also be constructed using a synthetic procedure. A polynucleotide can be synthesized manually or in a fully automated fashion. A polynucleotide can be a recombinant polynucleic acid. In some cases, a synthetic procedure may comprise 5′-hydroxyl oligonucleotides that can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof. Polynucleotides can be purchased commercially.

### a. Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides of the present disclosure. Details of the chemical synthesis are known in the art (e.g., Ganesan A. 2006 Mini Rev. Med. Chem. 6:3-10; and Camarero J. A. et al., 2005 Protein Pept Lett. 12:723-8).

### B. Recombinant Production

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. In some embodiments, eukaryotic cells that are used as host cells for production of the polypeptides include insect cells, mammalian cells, and/or plant cells. In some embodiments, mammalian host cells are used and may include human cells (e.g., HeLa, 293, H9, NSO, and Jurkat cells); mouse cells (e.g., NIH3T3, L cells, and C127 cells); primate cells (e.g., Cos 1, Cos 7 and CV1) and hamster cells (e.g., Chinese hamster ovary (CHO) cells). In specific embodiments, the polypeptide disclosed herein are produced in CHO cells.

In some embodiments, the host cells can be from a transgenic animal, e.g., mammary epithelial cell.

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding polynucleic acid. The polypeptide-encoding polynucleic acid can be provided as an inheritable episomal element (e.g., a plasmid) or can be genomically integrated and expressed under the control of an inducible promoter, for example. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences and may provide for inducible or constitutive expression where the coding region is operably-linked under the transcriptional control of the transcriptional initiation region, and a transcriptional and translational termination region. The transcriptional and translational regulatory sequences can include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7). A promoter disclosed herein can be a mammalian promoter or derived from a mammalian promoter. A promoter disclosed herein can be a human promoter or derived from a human promoter.

The promoter can be a promoter as found in a naturally-occurring genome. In some embodiments, a promoter is not found in a naturally-occurring genome. In some embodiments, the promoter is an engineered promoter. The promoter can be a minimal promoter.

Also provided herein are polynucleic acids encoding the polypeptides disclosed and/or guide RNA disclosed herein and/or Cas9 protein disclosed herein. In some embodiments, a polynucleic acid encoding the polypeptides disclosed herein is operably linked to a promoter sequence that confers expression of the polypeptide. In some embodiments, the sequence of the polynucleic acid is codon optimized for expression of the polypeptide in a human cell. In some embodiments, the polynucleic acid is a deoxyribonucleic acid (DNA). In some embodiments, the polynucleic acid is a ribonucleic acid (RNA). Also provided herein is a vector comprising the polynucleic acid encoding the polypeptides for binding a target polynucleic acid as described herein. In some embodiments, the vector is a viral vector.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

In some embodiments, a host cell comprising the polynucleic acid or the vector encoding the polypeptides disclosed herein is provided. In some embodiments, a host cell comprising the polypeptides disclosed herein is provided. In some embodiments, a host cell that expresses the polypeptide is also disclosed.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Delivery

Compositions comprising any polynucleic acid, cell, polypeptide, gene, gene product, or transcription factor described herein can be delivered by any suitable means. Compositions comprising a transcription factor (TF), a polynucleic acid encoding a TF, a polynucleic acid (e.g., in a CRISPR/Cas9 complex) for activating expression of a TF, polynucleic acids (e.g., in a CRISPR/Cas9 complex) for inhibiting expression of a TF can be delivered by any suitable means. The TF can be selected from, but are not limited to EGR1, ZFX, ATF4, VEZF1, MAZ, SOX2, PAX8, AFHX3, ATG4C, ATG5, HMGB1, STAT3, GATA2, and KLF4. The TF can be selected from, but are not limited to DLX6, E2F3, FOXM1, FOSL1, NFATC4, MYC, STAT4, GATA3, EZH2, HSF2, PAX4, NKX2-2, SIM2, and VAX1.

Compositions comprising E2F3, polynucleic acids encoding the disclosed E2F3 polynucleic acids (e.g., in a CRISPR/Cas9 complex) for activating expression of E2F3, polynucleic acids (e.g., in a CRISPR/Cas9 complex) for inhibiting expression of E2F3, and the like can be delivered into a cell by any suitable means, including, for example, by injection, infection, transfection, and vesicle or liposome mediated delivery.

Compositions comprising DLX6, polynucleic acids encoding the disclosed DLX6 polynucleic acids (e.g., in a CRISPR/Cas9 complex) for activating expression of DLX6, polynucleic acids (e.g., in a CRISPR/Cas9 complex) for inhibiting expression of DLX6, and the like can be delivered into a cell by any suitable means, including, for example, by injection, infection, transfection, and vesicle or liposome mediated delivery.

Compositions comprising FOSL1, polynucleic acids encoding the disclosed FOSL1 polynucleic acids (e.g., in a CRISPR/Cas9 complex) for activating expression of FOSL1, polynucleic acids (e.g., in a CRISPR/Cas9 complex) for inhibiting expression of FOSL1, and the like can be delivered into a cell by any suitable means, including, for example, by injection, infection, transfection, and vesicle or liposome mediated delivery.

Compositions comprising NFATC4, polynucleic acids encoding the disclosed NFATC4 polynucleic acids (e.g., in a CRISPR/Cas9 complex) for activating expression of NFATC4, polynucleic acids (e.g., in a CRISPR/Cas9 complex) for inhibiting expression of NFATC4, and the like can be delivered into a cell by any suitable means, including, for example, by injection, infection, transfection, and vesicle or liposome mediated delivery.

Compositions comprising MYC, polynucleic acids encoding the disclosed MYC polynucleic acids (e.g., in a CRISPR/Cas9 complex) for activating expression of MYC, polynucleic acids (e.g., in a CRISPR/Cas9 complex) for inhibiting expression of MYC, and the like can be delivered into a cell by any suitable means, including, for example, by injection, infection, transfection, and vesicle or liposome mediated delivery.

Compositions comprising STAT4, polynucleic acids encoding the disclosed STAT4 polynucleic acids (e.g., in a CRISPR/Cas9 complex) for activating expression of STAT4, polynucleic acids (e.g., in a CRISPR/Cas9 complex) for inhibiting expression of STAT4, and the like can be delivered into a cell by any suitable means, including, for example, by injection, infection, transfection, and vesicle or liposome mediated delivery.

Compositions comprising GATA3, polynucleic acids encoding the disclosed GATA3 polynucleic acids (e.g., in a CRISPR/Cas9 complex) for activating expression of GATA3, polynucleic acids (e.g., in a CRISPR/Cas9 complex) for inhibiting expression of GATA3, and the like can be delivered into a cell by any suitable means, including, for example, by injection, infection, transfection, and vesicle or liposome mediated delivery.

Compositions comprising EZH2, polynucleic acids encoding the disclosed EZH2 polynucleic acids (e.g., in a CRISPR/Cas9 complex) for activating expression of EZH2, polynucleic acids (e.g., in a CRISPR/Cas9 complex) for inhibiting expression of EZH2, and the like can be delivered into a cell by any suitable means, including, for example, by injection, infection, transfection, and vesicle or liposome mediated delivery.

Compositions comprising HSF2, polynucleic acids encoding the disclosed HSF2 polynucleic acids (e.g., in a CRISPR/Cas9 complex) for activating expression of HSF2, polynucleic acids (e.g., in a CRISPR/Cas9 complex) for inhibiting expression of HSF2, and the like can be delivered into a cell by any suitable means, including, for example, by injection, infection, transfection, and vesicle or liposome mediated delivery.

Compositions comprising PAX4, polynucleic acids encoding the disclosed PAX4 polynucleic acids (e.g., in a CRISPR/Cas9 complex) for activating expression of PAX4, polynucleic acids (e.g., in a CRISPR/Cas9 complex) for inhibiting expression of PAX4, and the like can be delivered into a cell by any suitable means, including, for example, by injection, infection, transfection, and vesicle or liposome mediated delivery.

Compositions comprising NKX2-2, polynucleic acids encoding the disclosed NKX2-2 polynucleic acids (e.g., in a CRISPR/Cas9 complex) for activating expression of NKX2-2, polynucleic acids (e.g., in a CRISPR/Cas9 complex) for inhibiting expression of NKX2-2, and the like can be delivered into a cell by any suitable means, including, for example, by injection, infection, transfection, and vesicle or liposome mediated delivery.

Compositions comprising SIM2, polynucleic acids encoding the disclosed SIM2 polynucleic acids (e.g., in a CRISPR/Cas9 complex) for activating expression of SIM2, polynucleic acids (e.g., in a CRISPR/Cas9 complex) for inhibiting expression of SIM2, and the like can be delivered into a cell by any suitable means, including, for example, by injection, infection, transfection, and vesicle or liposome mediated delivery.

Compositions comprising VAX1, polynucleic acids encoding the disclosed VAX1 polynucleic acids (e.g., in a CRISPR/Cas9 complex) for activating expression of VAX1, polynucleic acids (e.g., in a CRISPR/Cas9 complex) for inhibiting expression of VAX1, and the like can be delivered into a cell by any suitable means, including, for example, by injection, infection, transfection, and vesicle or liposome mediated delivery.

Compositions comprising EGR1, polynucleic acids encoding the disclosed EGR1 polynucleic acids (e.g., in a CRISPR/Cas9 complex) for activating expression of EGR1, polynucleic acids (e.g., in a CRISPR/Cas9 complex) for inhibiting expression of EGR1, and the like can be delivered into a cell by any suitable means, including, for example, by injection, infection, transfection, and vesicle or liposome mediated delivery.

Compositions comprising ZFX, polynucleic acids encoding the disclosed ZFX polynucleic acids (e.g., in a CRISPR/Cas9 complex) for activating expression of ZFX, polynucleic acids (e.g., in a CRISPR/Cas9 complex) for inhibiting expression of ZFX, and the like can be delivered into a cell by any suitable means, including, for example, by injection, infection, transfection, and vesicle or liposome mediated delivery.

Compositions comprising ATF4, polynucleic acids encoding the disclosed ATF4 polynucleic acids (e.g., in a CRISPR/Cas9 complex) for activating expression of ATF4, polynucleic acids (e.g., in a CRISPR/Cas9 complex) for inhibiting expression of ATF4, and the like can be delivered into a cell by any suitable means, including, for example, by injection, infection, transfection, and vesicle or liposome mediated delivery.

Compositions comprising VEZF1, polynucleic acids encoding the disclosed VEZF1 polynucleic acids (e.g., in a CRISPR/Cas9 complex) for activating expression of VEZF1, polynucleic acids (e.g., in a CRISPR/Cas9 complex) for inhibiting expression of VEZF1, and the like can be delivered into a cell by any suitable means, including, for example, by injection, infection, transfection, and vesicle or liposome mediated delivery.

Compositions comprising MAZ, polynucleic acids encoding the disclosed MAZ polynucleic acids (e.g., in a CRISPR/Cas9 complex) for activating expression of MAZ, polynucleic acids (e.g., in a CRISPR/Cas9 complex) for inhibiting expression of MAZ, and the like can be delivered into a cell by any suitable means, including, for example, by injection, infection, transfection, and vesicle or liposome mediated delivery.

Compositions comprising SOX2, polynucleic acids encoding the disclosed SOX2 polynucleic acids (e.g., in a CRISPR/Cas9 complex) for activating expression of SOX2, polynucleic acids (e.g., in a CRISPR/Cas9 complex) for inhibiting expression of SOX2, and the like can be delivered into a cell by any suitable means, including, for example, by injection, infection, transfection, and vesicle or liposome mediated delivery.

Compositions comprising PAX8, polynucleic acids encoding the disclosed PAX8 polynucleic acids (e.g., in a CRISPR/Cas9 complex) for activating expression of PAX8, polynucleic acids (e.g., in a CRISPR/Cas9 complex) for inhibiting expression of PAX8, and the like can be delivered into a cell by any suitable means, including, for example, by injection, infection, transfection, and vesicle or liposome mediated delivery.

Compositions comprising ZFHX3, polynucleic acids encoding the disclosed ZFHX3 polynucleic acids (e.g., in a CRISPR/Cas9 complex) for activating expression of ZF1X3, polynucleic acids (e.g., in a CRISPR/Cas9 complex) for inhibiting expression of ZFHX3, and the like can be delivered into a cell by any suitable means, including, for example, by injection, infection, transfection, and vesicle or liposome mediated delivery.

Compositions comprising ATG4C, polynucleic acids encoding the disclosed ATG4C polynucleic acids (e.g., in a CRISPR/Cas9 complex) for activating expression of ATG4C, polynucleic acids (e.g., in a CRISPR/Cas9 complex) for inhibiting expression of ATG4C, and the like can be delivered into a cell by any suitable means, including, for example, by injection, infection, transfection, and vesicle or liposome mediated delivery.

Compositions comprising ATG5, polynucleic acids encoding the disclosed ATG5 polynucleic acids (e.g., in a CRISPR/Cas9 complex) for activating expression of ATG5, polynucleic acids (e.g., in a CRISPR/Cas9 complex) for inhibiting expression of ATG5, and the like can be delivered into a cell by any suitable means, including, for example, by injection, infection, transfection, and vesicle or liposome mediated delivery.

Compositions comprising ATG5, polynucleic acids encoding the disclosed ATG5 polynucleic acids (e.g., in a CRISPR/Cas9 complex) for activating expression of ATG5, polynucleic acids (e.g., in a CRISPR/Cas9 complex) for inhibiting expression of ATG5, and the like can be delivered into a cell by any suitable means, including, for example, by injection, infection, transfection, and vesicle or liposome mediated delivery.

Compositions comprising HMGB1, polynucleic acids encoding the disclosed HMGB1 polynucleic acids (e.g., in a CRISPR/Cas9 complex) for activating expression of HMGB1, polynucleic acids (e.g., in a CRISPR/Cas9 complex) for inhibiting expression of HMGB1, and the like can be delivered into a cell by any suitable means, including, for example, by injection, infection, transfection, and vesicle or liposome mediated delivery.

Compositions comprising STAT3, polynucleic acids encoding the disclosed STAT3 polynucleic acids (e.g., in a CRISPR/Cas9 complex) for activating expression of STAT3, polynucleic acids (e.g., in a CRISPR/Cas9 complex) for inhibiting expression of STAT3, and the like can be delivered into a cell by any suitable means, including, for example, by injection, infection, transfection, and vesicle or liposome mediated delivery.

Compositions comprising GATA2, polynucleic acids encoding the disclosed GATA2 polynucleic acids (e.g., in a CRISPR/Cas9 complex) for activating expression of GATA2, polynucleic acids (e.g., in a CRISPR/Cas9 complex) for inhibiting expression of GATA2, and the like can be delivered into a cell by any suitable means, including, for example, by injection, infection, transfection, and vesicle or liposome mediated delivery.

Compositions comprising KLF4, polynucleic acids encoding the disclosed KLF4 polynucleic acids (e.g., in a CRISPR/Cas9 complex) for activating expression of KLF4, polynucleic acids (e.g., in a CRISPR/Cas9 complex) for inhibiting expression of KLF4, and the like can be delivered into a cell by any suitable means, including, for example, by injection, infection, transfection, and vesicle or liposome mediated delivery.

In certain embodiments, introducing a protein, polynucleic acid, or a small molecule for increasing or decreasing expression of a protein as disclosed herein can include administering a composition comprising the protein, polynucleic acid, or a small molecule to a human subject intradermally, e.g., by a superficial injection or topically, e.g., in a topical formulation.

### Vectors

Described herein, in certain embodiments, is a vector comprising one or more of the polynucleic acid molecules as described herein.

Further provided herein are vectors comprising the polypeptide sequences described herein. In some embodiments, the vectors comprise polynucleic acid sequences encoding the polypeptide sequences, described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

In some embodiments, a mRNA or a vector encoding the proteins disclosed herein may be injected, transfected, or introduced via viral infection into a cell, where the cell is ex vivo or in vivo. Any vector systems may be used including, but not limited to, plasmid vectors, retroviral vectors, lentiviral vectors, adenovirus vectors, poxvirus vectors: herpes simplex virus vectors and adeno-associated virus vectors, etc. Non-viral vector delivery systems include DNA plasmids, naked polynucleic acid, and polynucleic acid complexed with a delivery vehicle such as a liposome or poloxamer. Viral vector delivery systems include DNA and RNA viruses, which have either episomal or integrated genomes after delivery to the cell. Vectors suitable for introduction of polynucleotides as described herein include described herein include non-integrating lentivirus vectors (IDLV).

Non-viral vector delivery systems include electroporation, lipofection, microinjection, biolistics, virosomes, liposomes, immunoliposomes, polycation or lipid:nucleic acid conjugates, naked DNA, artificial virions, and agent-enhanced uptake of DNA.

Cells may be isolated and rejuvenated ex vivo for reintroduction into the subject.

In applications where transient expression of the polypeptide of the present disclosure is desired, adenoviral based systems may be used. Adeno-associated virus (“AAV”) vectors can also be used to transduce cells with polynucleic acids encoding the polypeptide of the present disclosure, e.g., in the in vitro production of polynucleic acids and peptides, and for in vivo and ex vivo gene therapy procedures. In some embodiments, recombinant adeno-associated virus vectors (rAAV) such as replication-deficient recombinant adenoviral vectors may be used for introduction of polynucleic acids encoding the polypeptides disclosed herein.

In some embodiments, polynucleic acids disclosed herein can be delivered using a gene therapy vector with a high degree of specificity to a particular tissue type or cell type. A viral vector is typically modified to have specificity for a given cell type by including a sequence encoding a ligand expressed as a fusion protein with a viral coat protein on the viruses' outer surface. The ligand is chosen to have affinity for a receptor known to be present on the cell type of interest.

In some embodiments, gene therapy vectors can be delivered in vivo by administration to an individual patient. In some embodiments, administration involves systemic administration (e.g., intravenous, intraperitoneal, intramuscular, subdermal, or intracranial infusion), direct injection (e.g., intrathecal), or topical application, as described below. Alternatively, vectors can be delivered to cells ex vivo, such as cells explanted from an individual patient followed by reimplantation of the cells into a patient, usually after selection for cells which have been rejuvenated.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the compositions as described herein to the fetus directly or indirectly (e.g., via the mother).

Suitable routes for administration of the compositions as described herein may vary depending on cell type to which delivery of the compositions is desired. The compositions as described herein may be administered to patients parenterally, for example, by intrathecal injection, intracerebroventricular injection, intraperitoneal injection, intramuscular injection, subcutaneous injection, or intravenous injection. The compositions as described herein may be administered to patients orally.

In some embodiments, the compositions as described herein are administered with one or more agents capable of promoting penetration of the subject the compositions as described herein across the blood-brain barrier by any method known in the art.

In some embodiments, the compositions as described herein can be administered using a nebulizer, inhaler, nasal spray, auto-injector, micro needle array, or eye drop.

In some embodiments, a delivery vector can be a non-viral vector. A delivery vector can be a lipid-based vector, for example, a proteo-lipid vehicle (PLV), a lipid nanoparticle (LNP), or a liposome. A lipid-based vector can comprise an electroneutral lipid. A lipid-based vector can comprise an ionizable lipid. A lipid-based vector can comprise a cationic lipid. A lipid-based vector can comprise, for example, 1,2-dioleoyl-3-dimethylammonium-propane (DODAP), 1,2-dioleoyl-3-trimethylammonium-propane (DOTAP), 1,2-dioleoyl-sn-glycero-3-phosphoethanolamine (DOPE), Cholesterol, 1,2-dimyristoyl-rac-glycero-3-methoxypolyethylene glycol (DMG-PEG), or any combination thereof.

### Compositions

In some embodiments, the polypeptides, the polynucleic acids, small molecules described herein may be present in a composition comprising a y acceptable excipient. In some embodiments, the polypeptides and the polynucleic acids are present in a therapeutically effective amount in the composition. A therapeutically effective amount can be determined based on an observed effectiveness of the composition. A therapeutically effective amount can be determined using assays that measure the desired effect in a cell. The compositions can be administered ex vivo or in vivo to a subject in order to practice the therapeutic and prophylactic methods and uses described herein.

The compositions of the present disclosure can be formulated to be compatible with the intended method or route of administration; exemplary routes of administration are set forth herein. Suitably acceptable or physiologically acceptable diluents, carriers or excipients include, but are not limited to, nuclease inhibitors, protease inhibitors, a suitable vehicle such as physiological saline solution or citrate buffered saline.

Certain aspects of the present disclosure relate to compositions and formulations (e.g., compositions and formulations) comprising any of the recombinant polynucleic acids (e.g., a recombinant viral genome such as herpes virus genome) and/or viruses (e.g., rAAV virus, a herpes virus comprising a recombinant genome described herein (such as a herpes simplex virus comprising a recombinant herpes simplex virus genome), and an excipient or carrier (e.g., a suitably acceptable excipient or carrier). In some embodiments, the composition or formulation is a cosmetic composition or formulation (e.g., a skin care product) for example in the form of a liquid formulation such as, serum, astringent, or a fluid such as a cream or a lotion.

In some embodiments, the composition provided herein comprises an activator of a transcription factor of DLX6, E2F3, FOXM1, FOSL1, NFATC4, MYC, STAT4, GATA3, EZH2, HSF2, PAX4, NKX2-2, SIM2, and/or VAX1. In some embodiments, the composition provided herein comprises a inhibitor of a transcription factor of EGR1, ZFX, ATF4, VEZF1, MAZ, SOX2, PAX8, ZFHX3, ATG4C, ATG5, HMGB1, STAT3, GATA2, and/or KLF4.

In some embodiments, the composition does not comprise an activator of a transcription factor of OCT4, SOX2, KLF4, or MYC.

In some embodiments, the activator comprises a polynucleic acid encoding the transcription factor or an expression plasmid comprising the polynucleic acid encoding the transcription factor.

In some embodiments, the inhibitor comprises a CRISPR system, a repression plasmid, an antisense oligonucleotide, or siRNA; or a polynucleic acid encoding the CRISPR system, the antisense oligonucleotide, or the siRNA.

### Cells

Also provided herein is a cell or cells comprising an activator of a transcription factor. The transcription factor can be selected from, but are not limited to DLX6, E2F3, FOXM1, FOSL1, NFATC4, MYC, STAT4, GATA3, EZH2, HSF2, PAX4, NKX2-2, SIM2, and VAX1. The activator of a transcription factor can be exogenous. The activator of a transcription factor can be a polynucleic acid encoding the transcription factor or a vector comprising the polynucleic acid.

Also provided herein is a cell or cells comprising an inhibitor of a transcription factor. The transcription factor can be selected from, but are not limited to EGR1, ZFX, ATF4, VEZF1, MAZ, SOX2, PAX8, ZFHX3, ATG4C, ATG5, HMGB1, STAT3, GATA2, and KLF4. The inhibitor of a transcription factor can be exogenous. The inhibitor of a transcription factor can be a CRISPR system, a repression plasmid, an antisense oligonucleotide, or siRNA targeting the transcription factor. The inhibitor of a transcription factor can be a polynucleic acid encoding the CRISPR system, the antisense oligonucleotide, or the siRNA targeting the transcription factor, or a vector comprising the polynucleic acid.

Also provided herein is a cell or cells comprising an activator of a transcription factor and an inhibitor of a transcription factor. The transcription factor can be selected from, but are not limited to DLX6, E2F3, FOXM1, FOSL1, NFATC4, MYC, STAT4, GATA3, EZH2, HSF2, PAX4, NKX2-2, SIM2, VAX1, EGR1, ZFX, ATF4, VEZF1, MAZ, SOX2, PAX8, ZFHX3, ATG4C, ATG5, HMGB1, STAT3, GATA2, and KLF4.

In some embodiments, the cell is produced by the method described herein. In some embodiments, the cell can be a non-human cell, a human cell, an adult human cell, an old cell, a young cell, a mammalian cell, or a eukaryotic cell.

In some embodiments, the cell is a differentiated cell, e.g., skin cell, lung cell (e.g., lung fibroblast), liver cell (e.g., hepatocyte), muscle cell (e.g., cardiac muscle cell), pancreatic cell, immune cell, bone cell, brain cell (e.g., microglial cell, glial cell, astrocytes, neurons, etc.), eye cell (e.g., retinal cell, such as, retinal cell is a retinal ganglion cell, an amacrine cell, a horizontal cell, a bipolar cell, a photoreceptor cell, a Müller glial cell, a microglial cell, or a retinal pigmented epithelium cell), scalp cell (e.g., hair follicle), etc. In certain embodiments, the cell may be fibroblast, e.g., skin fibroblast, liver fibroblast, lung fibroblast, or skeletal muscle fibroblast. In certain embodiments, the cell may be pancreatic islet cell. In certain embodiments, the cell may be T cell, B cell, macrophages, or dendritic cell. In certain embodiments, the cell may be bone marrow cell. In certain embodiments, the cell may be neural cell, glial cell, or astrocyte.

### Pharmaceutical Compositions

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

A pharmaceutical composition of the disclosure can comprise a therapeutic complex of the present disclosure. A pharmaceutical composition can be a combination of any therapeutic complexes described herein with other chemical components, such as carriers, stabilizers, diluents, dispersing agents, suspending agents, thickening agents, and/or excipients. The pharmaceutical composition facilitates administration of the therapeutic complex to an organism.

Pharmaceutical formulations for administration can include aqueous solutions of the active composition in water-soluble form. Suspensions of the active composition can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compositions to allow for the preparation of highly concentrated solutions. The active ingredient can be in powder form for constitution with a suitable vehicle, for example, sterile pyrogen-free water, before use.

The pharmaceutical compositions can include at least one pharmaceutically-acceptable carrier, diluent, or excipient and compositions described herein as free-base or pharmaceutically-acceptable salt form.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A therapeutic complex described herein can be conveniently formulated into pharmaceutical compositions composed of one or more pharmaceutically-acceptable carriers. See e.g., Remington's Pharmaceutical Sciences, latest edition, by E. W. Martin Mack Pub. Co., Easton, PA, incorporated by reference in its entirety, which discloses typical carriers and conventional methods of preparing pharmaceutical compositions. Such carriers can be carriers for administration of compositions to humans and non-humans, including solutions such as sterile water, saline, and buffered solutions at physiological pH. Pharmaceutical compositions can also include one or more additional active ingredients such as antimicrobial agents, anti-inflammatory agents, and anesthetics.

Non-limiting examples of pharmaceutically-acceptable carriers include saline, Ringer's solution, and dextrose solution. In some embodiments, the pH of the solution can be from about 5 to about 8, and can be from about 7 to about 7.5. Further carriers include sustained release preparations such as semipermeable matrices of solid hydrophobic polymers containing the therapeutic complex. The matrices can be in the form of shaped articles, for example, films, liposomes, microparticles, or microcapsules.

Non-limiting examples of pharmaceutically active agents suitable for combination with compositions of the disclosure include anti-infectives, i.e., aminoglycosides, antiviral agents, antimicrobials, anti-cholinergics/anti-spasmotics, antidiabetic agents, antihypertensive agents, anti-neoplastics, cardiovascular agents, central nervous system agents, coagulation modifiers, hormones, immunologic agents, immunosuppressive agents, and ophthalmic preparations.

In some embodiments, the pharmaceutical composition provided herein comprises a therapeutically effective amount of a therapeutic complex herein in admixture with a pharmaceutically-acceptable carrier and/or excipient, for example, saline, phosphate buffered saline, phosphate and amino acids, polymers, polyols, sugar, buffers, preservatives, and other proteins. Illustrative agents include octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene, and glycol.

In some embodiments, a pharmaceutical formulation disclosed herein can comprise: (i) a therapeutic complex disclosed herein; (ii) a buffer; (iii) a non-ionic detergent; (iv) a tonicity agent; and (v) a stabilizer. In some embodiments, the pharmaceutical formulation disclosed herein is a stable liquid pharmaceutical formulation.

In some embodiments, a pharmaceutical formulation disclosed herein is a liquid formulation that can comprise about 5 mg/mL to about 150 mg/mL of the therapeutic complex, about 7.5 mg/mL to about 140 mg/mL of the therapeutic complex, about 10 mg/mL to about 130 mg/mL of the therapeutic complex, about 10 mg/mL to about 100 mg/mL of the therapeutic complex, about 20 mg/mL to about 80 mg/mL of the therapeutic complex, or about 30 mg/mL to about 70 mg/mL of the therapeutic complex. For example, a formulation of the present disclosure can comprise about 5 mg/mL, about 10 mg/mL, about 15 mg/mL, about 20 mg/mL, about 25 mg/mL, about 30 mg/mL, about 35 mg/mL, about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, or about 150 mg/mL of a therapeutic complex described herein.

In some embodiments, a pharmaceutical formulation disclosed herein can comprise a buffer. In some embodiments, the buffer serves to maintain a stable pH and to help stabilize a therapeutic complex disclosed herein. In some embodiments, the buffer or buffer system comprises at least one buffer that has a buffering range that overlaps fully or in part the range of pH 5.5-7.4. In some embodiments, the buffer has a pKa of about 6.2±0.5. In some embodiments, the buffer comprises a sodium phosphate buffer. In some embodiments, the sodium phosphate is present at a concentration of about 5 mM to about 15 mM, about 6 mM to about 14 mM, about 7 mM to about 13 mM, about 8 mM to about 12 mM, about 9 mM to about 11 mM, or about 10 mM. In certain embodiments, the buffer system comprises sodium phosphate at 10 mM, at a pH of 6.2±0.3 or 6.1±0.3.

The pH of the disclosed composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. A pharmaceutical formulation disclosed herein can have a pH of from about 5.5 to about 6.5. For example, a formulation of the present disclosure can have a pH of about 5.5, about 5.6, about 5.7, about 5.8, about 5.9, about 6.0, about 6.1, about 6.2, about 6.3, about 6.4, or about 6.5. In some embodiments, the pH is 6.2±0.3, 62±0.2, 6.2±0.1, about 6.2, or 6.2.

If the pH is outside the range desired by the formulator, the pH can be adjusted by using sufficient pharmaceutically-acceptable acids and bases.

In some embodiments, a pharmaceutical formulation disclosed herein can comprise a non-ionic detergent. In some embodiments, the non-ionic detergent is a nonionic polymer containing a polyoxyethylene moiety. In some embodiments, the non-ionic detergent is any one or more of polysorbate 20, poloxamer 188 or polyethylene glycol 3350. In some embodiments, the non-ionic detergent is polysorbate 20. In some embodiments, the non-ionic detergent is polysorbate 80. In some embodiments, a pharmaceutical formulation disclosed herein can contain about 0.01% to about 1% non-ionic detergent. For example, a formulation of the present disclosure can comprise about 0.0085%, about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.3%, about 0.4%, about 0.5%, about 0.6%, about 0.7%, about 0.8%, about 0.9%, about 1%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, or about 2% polysorbate 20, polysorbate 80 or poloxamer 188.

In some embodiments, a pharmaceutical formulation disclosed herein can comprise a tonicity agent. In some embodiments, the tonicity agent is sodium chloride or potassium chloride. In some embodiments, the tonicity agent is sodium chloride. In some embodiments, the sodium chloride is present at a concentration of about 5 mM to about 100 mM, about 10 mM to about 50 mM, or about 40 mM.

In some embodiments, a pharmaceutical formulation disclosed herein can comprise a stabilizer. In some embodiments, the stabilizer is a thermal stabilizer that can stabilize a therapeutic complex disclosed herein under conditions of thermal stress. In some embodiments, the stabilizer maintains greater than about 93% of the therapeutic complex in a native conformation when the solution containing the therapeutic complex and the thermal stabilizer is kept at about 45° C. for up to about 28 days. In some embodiments, the stabilizer prevents aggregation of the therapeutic complex and less than 4% of the therapeutic complex is aggregated when the solution containing the therapeutic complex and the thermal stabilizer is kept at about 45° C. for up to about 28 days. In some embodiments, the stabilizer maintains greater than about 96% of the therapeutic complex in a native conformation when the solution containing the therapeutic complex and the thermal stabilizer is kept at about 37° C. for up to about 28 days. In some embodiments, the stabilizer prevents aggregation of the therapeutic complex and less than about 2% of the therapeutic complex is aggregated when the solution containing the therapeutic complex and the thermal stabilizer is kept at about 37° C. for up to about 28 days.

In some embodiments, the thermal stabilizer is a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol or trehalose. In some embodiments, the stabilizer is sucrose. In some embodiments, a pharmaceutical formulation or ophthalmic formulation disclosed herein can comprise about 1% to about 20% sugar or sugar alcohol, about 2% to about 18% sugar or sugar alcohol, about 3% to about 15% sugar or sugar alcohol, about 4% to about 10% sugar or sugar alcohol, or about 5% sugar or sugar alcohol. For example, a pharmaceutical formulation or ophthalmic formulation of the present disclosure can comprise about 4%, about 5%, about 6%, about 7%, about 8%, about 9%, about 10%, about 11%, about 12%, about 13%, or about 14% sugar or sugar alcohol (e.g., sucrose, trehalose or mannitol). In some embodiments, the stabilizer is at a concentration of from about 1% w/v to about 20% w/v. In some embodiments, the stabilizer is sucrose at a concentration of from about 1% w/v to about 15% w/v, or from about 1% w/v to about 10% w/v. In some embodiments, the stabilizer is sucrose at a concentration of 5% w/v or about 5% w/v. In some embodiments, the stabilizer is sucrose at a concentration of 7.5% w/v or about 7.5% w/v. In some embodiments, the stabilizer is sucrose at a concentration of 10% w/v or about 10% w/v. In some embodiments, the stabilizer is sucrose at a concentration of 12.5% w/v or about 12.5% w/v. In some embodiments, the stabilizer is sucrose at a concentration of 15% w/v or about 15% w/v. In some embodiments, the stabilizer is sucrose at a concentration of 20% w/v or about 20% w/v.

A therapeutic complex of the disclosure can be, for example, an immediate release form or a controlled release formulation. An immediate release formulation can be formulated to allow the therapeutic complex to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements, or has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a therapeutic complex's action for an extended period of time. A delayed release form can be formulated to delay the release of an effective dose of one or more therapeutic complexes, for example, for about 4, about 8, about 12, about 16, or about 24 hours.

A controlled release formulation can be a sustained release form. A sustained release form can be formulated to sustain, for example, the therapeutic complex's action over an extended period of time. A sustained release form can be formulated to provide an effective dose of any therapeutic complex described herein (e.g., provide a physiologically-effective blood profile) over about 4, about 8, about 12, about 16, or about 24 hours.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s): pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Rejuvenation of Human Cells

A method to rejuvenate human cells via gene targets is provided. When the expression of any one of four genes was changed, human in vitro aged cells were rejuvenated by alleviating many aging phenotypes and altering global gene expression back to a “young state”. Until now, the only genes known to rejuvenate human cells are four stem cell genes known as the Yamanaka factors. Discovery of genes capable of rejuvenating cells is disclosed. When the gene expression of any of these genes was changed, many markers of cell health were improved: healthier protein recycling, more cell division, more mitochondria expression, and/or less senescence. This rejuvenation was independent of classic epigenetic changes and telomere length. Altering the expression of these new genes did not cause cancer or dedifferentiation, which are two serious issues with the rejuvenation done via Yamanaka factors.

Provided herein are other gene target solutions to transcriptional rejuvenation, and some of the solutions may be safer than reprogramming via the Yamanaka factors. In particular, the TFs disclosed herein can rejuvenate cells based on their ability to significantly change the expression of many genes at once. Experiments were designed to find TFs capable of rejuvenating cells, which cause global shifts in gene expression back to a younger state and alleviating several cellular aging hallmarks.

To model aging, early and late passage neonatal primary dermal skin fibroblasts (modeling young and old respectively) were used because these cells display aspects of both aging and senescence. These cells are referred to as early passage fibroblasts/cells and late passage fibroblasts/cells from now on. Senescence—when cells that ordinarily grow and divide stop dividing forever—increases with age and in late passage cells. It is a relevant marker of both in vitro and in vivo cellular aging. Both senescence markers and other common aging markers manifest gradually, starting many population doublings (PDs) before cells reach senescence.

To find TFs capable of rejuvenating late passage cells, a high-throughput Perturb-seq screen (Adamson, B. et al. A Multiplexed Single-Cell CRISPR Screening Platform Enables Systematic Dissection of the Unfolded Protein Response. Cell 167, 1867-1882.e21 (2016)) was performed of 200 TFs, selected via computational and literature searches. First, the 200 TFs were individually overexpressed with CRISPR activation (CRA) or individually repressed with CRISPR inhibition (CRI) in late passage fibroblasts. Then, changes in those cells' gene expression were measured via single cell RNA-seq (scRNA-seq). Single cell RNA-seq assayed the global transcriptional state of cells and thus identified TF perturbations which changed the late passage cells back to an earlier passage state. Screening using this high dimensional phenotype is thus superior to screening based on a single or a few reporters.

From these 400 total TF perturbations in late passage cells, computational analyses were performed to identify the perturbations that best rejuvenated these late passage cells using single cell RNA sequencing data, followed by cell assays to validate the rejuvenation by various aging markers. Four TF perturbations were found to consistently reverse late passage gene expression and cellular aging phenotypes to an earlier passage state. CRA cells overexpressing EZH2 or E2F3, and CRI cells repressing STAT3 or ZFX reversed aging phenotypes (referred as CRA EZH2, CRA E2F3, CRI STAT3, and CRI ZFX, respectively). These TF perturbations caused similar cellular rejuvenation phenotypes as the Yamanaka factors, but without causing dedifferentiation or cancer. When any of these TFs were targeted in late passage cells, the cells had more cell division, less senescence, improved proteostasis, and more mitochondrial expression as compared to the control cells: no cocktail of gene perturbations was required.

Selecting Candidate Transcription Factors with Single Cell RNA Sequencing Data from WT Passaged Fibroblasts

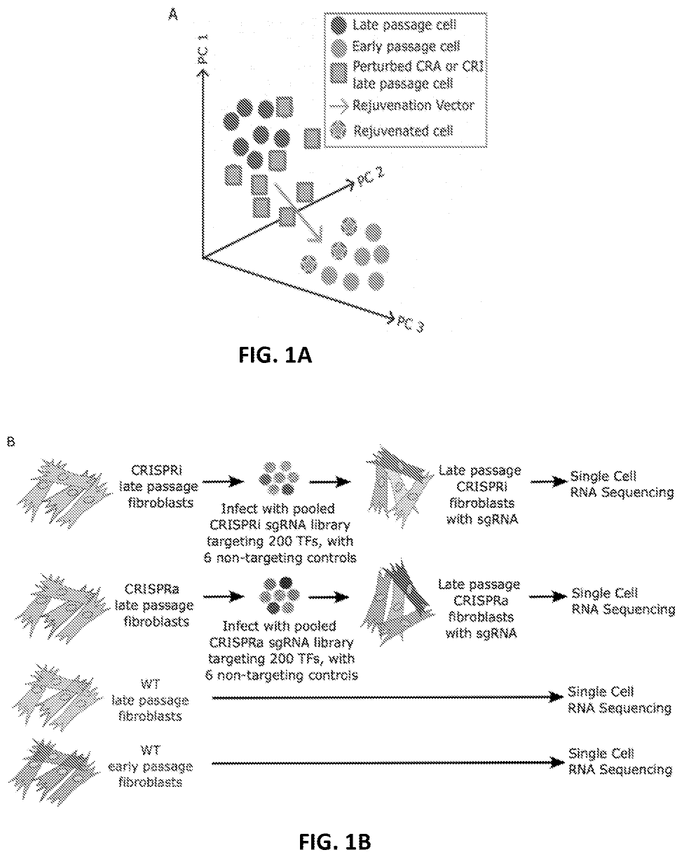

Experiments were designed to find TF perturbations capable of rejuvenating late passage fibroblasts. Rejuvenation was defined as reversing late passage cell gene expression and phenotypes back to an earlier passage state. FIG. 1A depicts the approach used herein, where each point in the high dimensional gene expression space represents one cell. Late passage and early passage wild-type (WT) cells cluster apart from each other due to their gene expression differences. Most late passage cells with TF perturbations will also cluster near WT late passage cells. However, cells with rejuvenating TF perturbations will shift towards the early passage WT cells.

To define the young and old states, first, the gene expression of passaged WT fibroblasts was measured using single-cell RNA sequencing (scRNA-seq). Based on preliminary cell experiments and gene expression data, the cells were categorized as follows: WT cells from population doubling (PD) 1-19 are early passage, cells from PD 20-30 are middle passage, and cells from PD 30-39 are late passage. At approximately PD 40, cells become fully senescent. Gene expression patterns largely recapitulated previous fibroblast and cellular aging data (López-Otín, C., Blasco, M. A., Partridge, L., Serrano, M. & Kroemer, G. The hallmarks of aging. Cell 153, 1194-1217 (2013); Tigges, J. et al. The hallmarks of fibroblast ageing. Mechanisms of Ageing and Development vol. 138 26-44 (2014); Chan, M. et al. Novel insights from a multiomics dissection of the Hayflick limit. Elife 11, (2022)). For example, late passage cells had lower gene expression related to the cell cycle, mitochondria, proteasome, and ribosome biogenesis. Late passage cells had more gene expression related to secretory pathways, extracellular matrix, and senescence. Using this data, candidate TFs were identified.

scRNA-seq was performed on early, middle, and late passage of WT fibroblasts and analyzed the gene pathway differences among them. Patterns largely recapitulated previous fibroblast and cellular aging Late passage cells had less gene expression related to cell cycle, mitochondria, proteasome, ribosome biogenesis, and splicing. Late passage cells had more gene expression related to secretory pathways, extracellular matrix, and senescence. This information was used to (1) identify transcription factors playing a role in these gene expression differences, (2) alter the expression of these TFs in late passage cells, and (3) calculate which perturbations reversed global gene expression back to an earlier passage state.

There are approximately 1500 human transcription factors, cofactors, and chromatin regulators. 200 of these TFs were investigated, overexpressing and repressing each one, in late passage fibroblasts. TFs were selected through a combination of computational analysis (⅔rds of the TFs) and literature searches (⅓rd of the TFs). For the computational TF selection, the lab's computational TF prediction tool was used, where gene expression data was added from early and late passage skin fibroblasts and known TF binding motifs. The tool outputs a ranked list of the TFs most likely causing the gene expression differences. For literature searches, TFs with links to senescence and cellular aging were identified.

To alter the expression of hundreds of TFs individually and in a high-throughput manner, stable cell lines were created in late passage fibroblasts expressing either CRISPR interference (CRI) and CRISPR activation (CRA) to repress or overexpress target TFs, respectively. Then, these cells were infected with a pooled guide RNA (sgRNA) library targeting these 200 TFs (Horlbeck, M. A. et al. Compact and highly active next-generation libraries for CRISPR-mediated gene repression and activation. Elife 5, (2016)) along with six non-targeting control guides, and performed scRNA-seq (Adamson, B. et al. A Multiplexed Single-Cell CRISPR Screening Platform Enables Systematic Dissection of the Unfolded Protein Response. Cell 167, 1867-1882.e21 (2016); Replogle, J. M. et al. Combinatorial single-cell CRISPR screens by direct guide RNA capture and targeted sequencing. Nat. Biotechnol. 38, 954-961 (2020)). These data assay the global gene expression changes in these perturbed cells and allow for identification of which TFs reversed gene expression back to an early passage state (FIG. 1B).

### Identifying Top Four Rejuvenating Transcription Factors Through Computational Analyses and Cell Assays

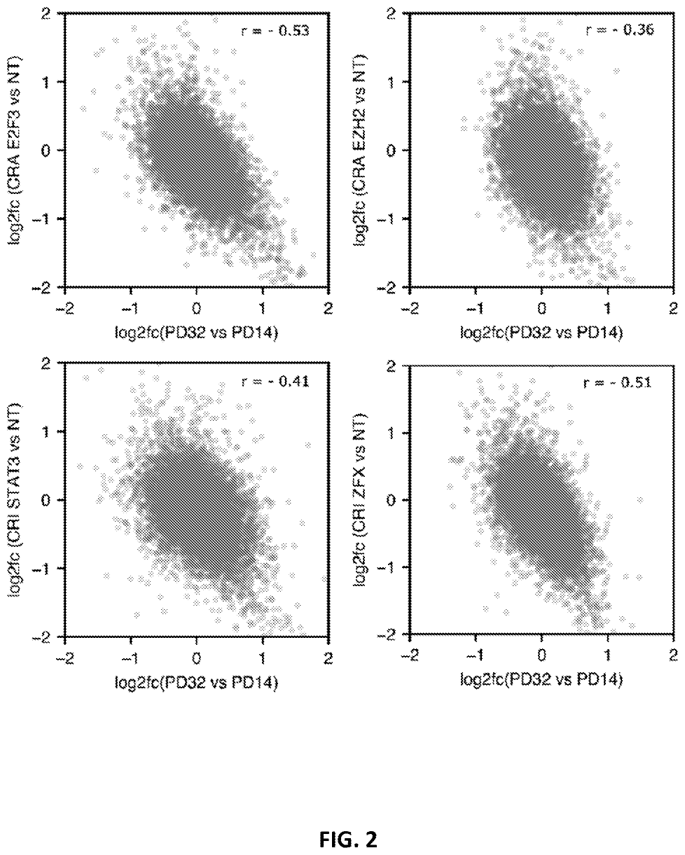

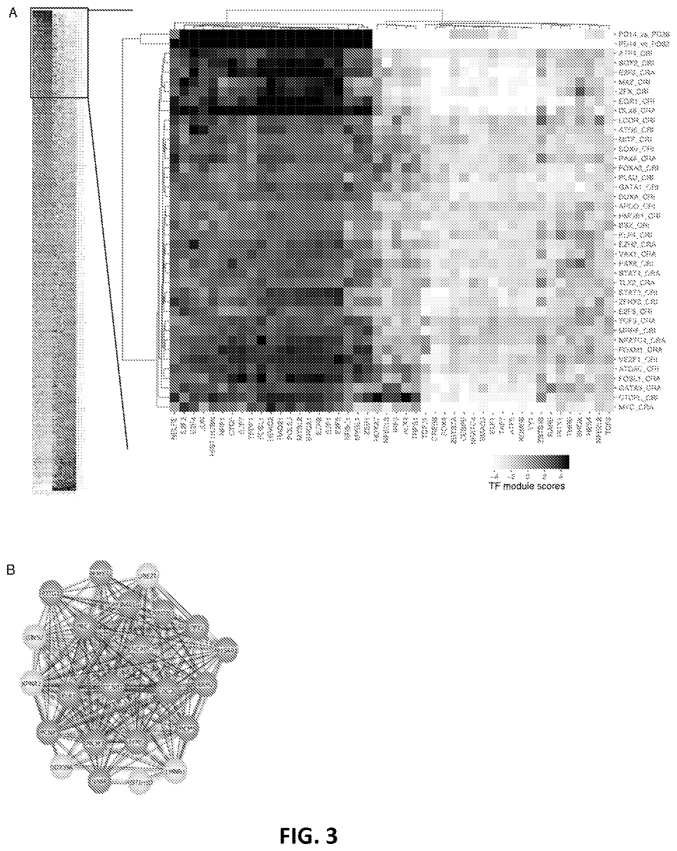

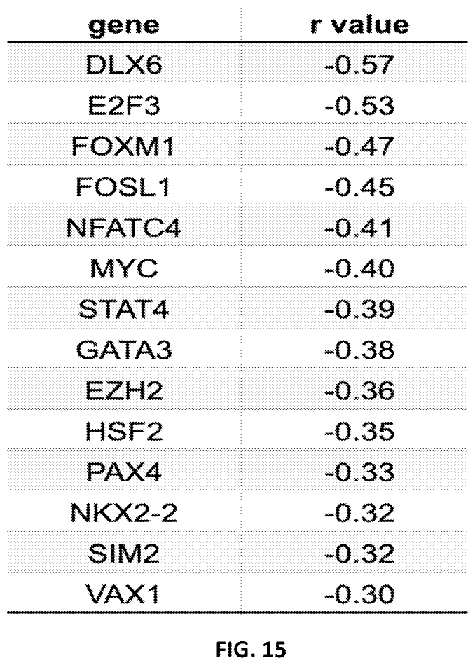

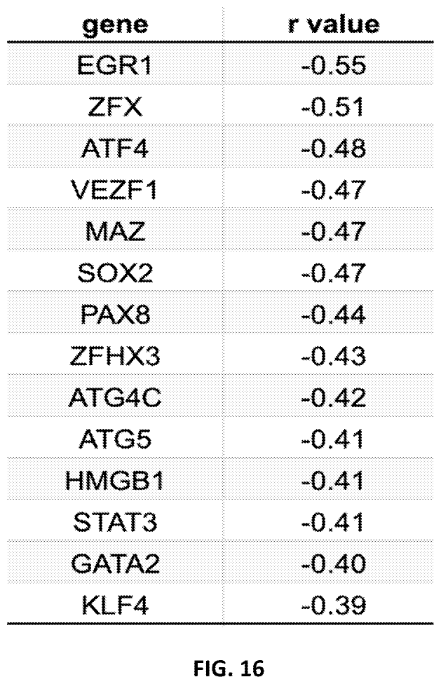

For each TF perturbation, the gene expression differences were calculated between late passage cells with a TF perturbed versus late passage cells with non-targeting (NT) sgRNAs (control cells). These gene expression differences were then compared with those between WT (no CRISPR construct) late passaged cells and WT early passaged cells. TF perturbations with a significant negative correlation (as measured by the Pearson correlation coefficient r-rejuvenation) indicated the perturbation reversed the gene expression changes due to replicative aging (FIG. 2). From the 200 overexpressed and repressed TFs, 28 TF perturbations were identified with strong negative r-rejuvenation (see the top 14 hits for CRA and CRI in FIGS. 15 and 16, and examples of the global gene expression changes in FIG. 3), suggesting that late passaged fibroblast cells may be rejuvenated by targeting these TFs.

TF perturbations reversed gene expression in late passage cells back towards an earlier passage state (FIG. 2). Correlation plots comparing gene expression changes between late passage and early passage WT cells to that between late passage cells with a TF perturbation and those with the NT control. Shown are CRA E2F3, CRA EZH2, CRI STAT3, and CRI ZFX, these TF perturbations that were subsequently extensively validated with cellular and molecular phenotyping. TF perturbations with a significant negative correlation (as measured by the Pearson correlation coefficient r-value) indicated that the TF perturbation reversed gene expression changes due to replicative aging.

From 400 TF perturbations, the list was narrowed to a top 28 TF perturbations, based upon the TF's correlation r-value (FIG. 15 and FIG. 16). From these 28 TFs, preliminary follow-up experiments were performed, including investigating fold change of the TF by CRI or CRA, the effects on cell cycle (Wolf, F. A., Angerer, P. & Theis, F. J. SCANPY: large-scale single-cell gene expression data analysis. Genome Biol. 19, 15 (2018)), and gene ontology analysis of classic aging and senescence pathways. This demonstrated that many TF perturbations actually rewired gene regulatory networks towards an earlier passage state (FIG. 3). The AUCell (Area Under the Curve) t-test scores for selected TF modules were analyzed via pySCENIC (Aibar, S. et al. SCENIC: single-cell regulatory network inference and clustering. Nat. Methods 14, 1083-1086 (2017); Van de Sande, B. et al. A scalable SCENIC workflow for single-cell gene regulatory network analysis. Nat. Protoc. 15, 2247-2276 (2020)) and it was observed that TF perturbations with large negative correlation r-values often caused similar gene regulatory networks to shift towards being similar to early passage WT cells. These data indicated that (1) the passaged fibroblasts can have tightly interconnected networks of genes (2) perturbing different TFs can lead to similar gene expression outcomes.

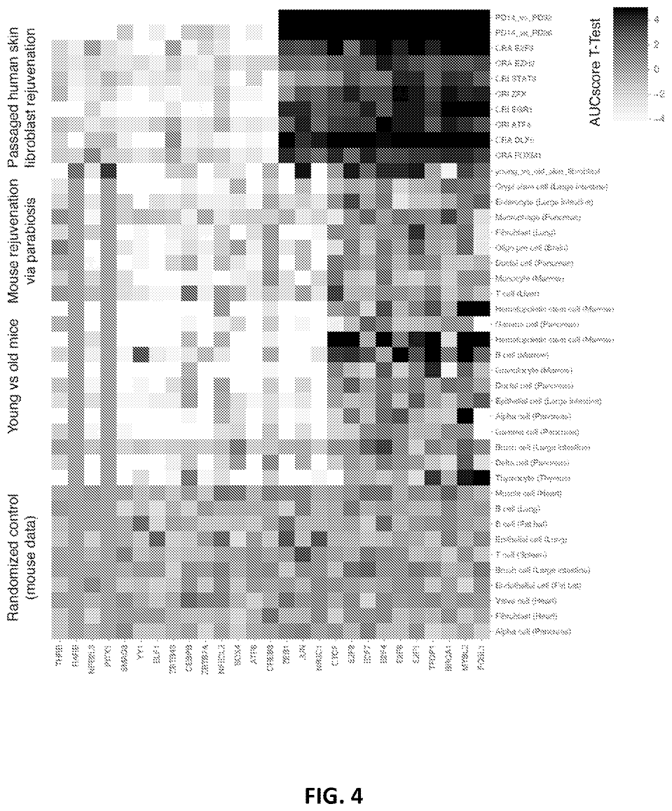

The convergent downstream transcriptional profiles from multiple rejuvenating TF perturbations define a transcriptional signature of passaged fibroblast rejuvenation. The next aim was to determine whether such a signature is observed in other aging and rejuvenation models. The TF module profiles were then analyzed for in vivo aging of human skin fibroblasts (comparing young and old patient skin fibroblast cells (See Zou, Dev. Cell, 56 383-397.e8 (2021)) and in mouse rejuvenation through parabiosis (See Palovics, Nature 603, 309-314 (2022)). A similar transcriptional signature was observed in skin fibroblasts from young patients relative to the old patients and in various tissues and cell types from old mice that were rejuvenated through parabiosis (FIG. 4). For example, there is a set of TF modules less expressed in early passage fibroblasts and in rejuvenating TF perturbations that were also less expressed in various tissue/cell types in old mice that underwent parabiosis. These TF modules have a broad range of functions. Examples include ATF6, which is a major transcriptional regulator of ER unfolded protein response, SMAD3, which is an intracellular signal transducer and transcriptional modulator activated by the TGF-beta signaling pathway, and YYl, which is a multifunctional transcription factor that exhibits positive and negative control on a large number of genes. Interestingly, tissue/cell types in rejuvenated mice that exhibit this transcriptional signature were enriched for mitotically active cells (such as large intestine crypt stem cells, pancreas ductal cells, lung fibroblast, and hematopoietic stem cells). These data suggest a common set of molecular requirements for rejuvenation, perhaps of proliferative cells, across species, cell types, and different rejuvenation methods.

From these initial experiments and computational analyses, these TF perturbations were identified which consistently reversed aging phenotypes in late passage cells: CRA EZH2, CRA E2F3, CRI STAT3 and CRI ZFX. For the original 200 TF screen, EZH2 and STAT3 were selected from literature searches, while E2F3 and ZFX were selected by the TF prediction tool. E2F3 is largely related to cell cycle. EZH2 is a methyl-transferase best known for being a part of the polycomb repressive complex 2 (PRC2) but has other roles outside the PRC2 complex. STAT3 is best known in fibroblasts for its role in inflammation. And, ZFX is relatively poorly understood TF, but there are links to stem cell renewal. Targeting any one of these TFs was found to alleviate aging phenotypes across diverse pathways. EZH2 is especially intriguing. EZH2 was found to be upregulated in most cells with rejuvenating TF perturbations (large negative r-values). Repressing EZH2 with CRI was one of the most “aging” TF perturbations (largest positive correlation).

Additionally, it was found that global gene expression reverted back to an earlier passage stage in all TF perturbations. Hallmarks of cellular aging that were rejuvenated by these TF perturbations were measured. Through cell aging experiments, diverse cell aging hallmarks were found to be alleviated in all TF perturbations.

Transcription factor module analysis revealed rejuvenating TF perturbations drive similar downstream gene expression changes (FIG. 3). TF perturbations (rows) were clustered by the AUC t-test scores for selected TF modules (columns) from the SCENIC analysis. Only the modules differentially expressed between WT PD 14 (early passage) and PD 32 (late passage) (|t-test score|>5) were shown. A positive module score means the genes in that TF module were more expressed in the TF perturbation compared to the NT control, or earlier passage cells compared to later passage cells, and a negative module score means the genes in that TF module were less expressed in the TF 38 perturbation or earlier passage cells. The AUC t-test score is derived from the AUC score for individual cells from the SCENIC analysis. Interaction network analysis (using string-db) was performed of commonly up-regulated genes within a cluster of TF perturbations (CRA E2F3, CRA DLX6, CRI ZFX, CRI EGR1, CRI MAZ, CRI SOX2, and CRI ATF4). The lines connecting the circles indicate interactions or connections between genes (FIG. 3).

Transcriptional signature of rejuvenation was found to be shared across species, cell types, and rejuvenation methods (FIG. 4). The AUC t-test score was derived from the AUC score for individual cells from the SCENIC analysis. Rows are organized into five blocks. From top to bottom are comparisons between: 1) early passage and late passage human fibroblasts, and rejuvenating TF perturbations and NT control; 2) skin fibroblasts from young and old patients; 3) mice rejuvenated via parabiosis (aged mice from the aged-young pairs) and control mice (aged mice from aged-aged pair), for the tissues/cell types indicated; 4) young mice (from the young-young pair) and old mice (from the old-old pair); 5) randomized controls, where for a given tissue/cell type, the two groups of cells were merged and randomly repartitioned into two groups of the same sizes. For block 1, two pairs of passaged cells and eight representative TF perturbations were selected. For blocks 3 to 5, the top 10 hits (those most similar to PD14 vs. PD32 profile based on Euclidean distance) were selected. Human TF modules were derived from the corresponding single cell data using SCENIC. Mouse TF modules were obtained through ortholog mapping of genes in the human TF modules (see Methods). Only the modules differentially expressed between WT PD 14 (early passage) and PD 32 (late passage) (|t-test score|>5) were shown (FIG. 4).

### TF Perturbations Increased Cell Division Rates and Decreased Senescence

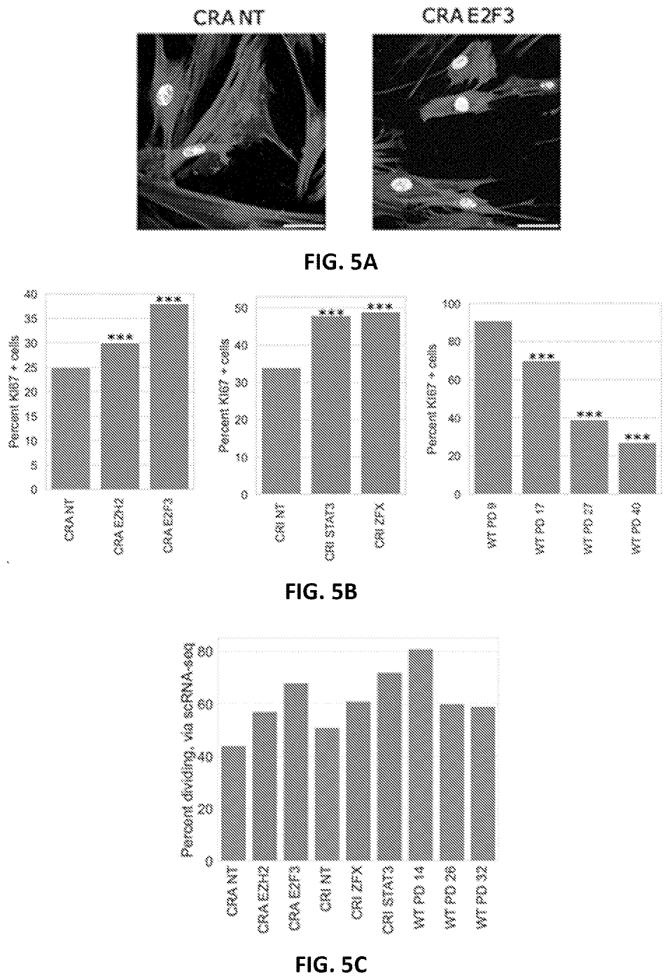

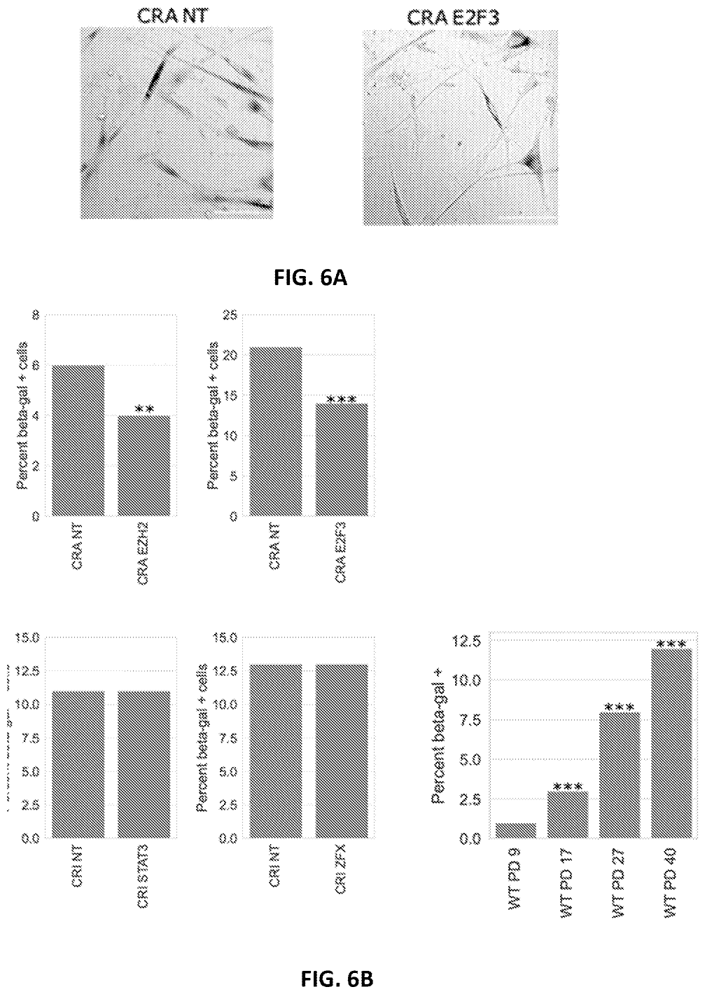

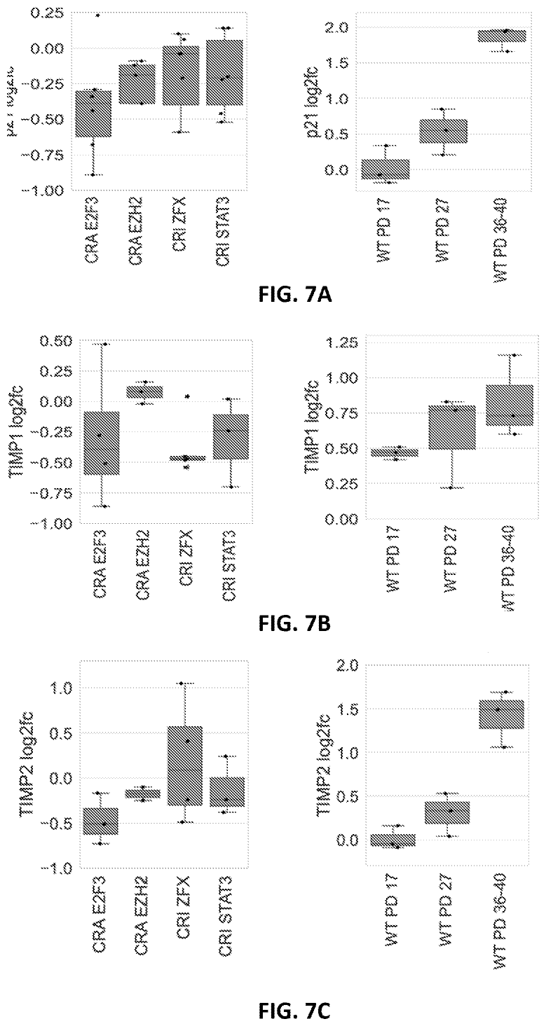

Late passage cells divide significantly less often and have more senescence than early passage cells. Experiments were performed to determine whether these TF perturbations rejuvenated cell division and senescence levels back to an earlier passage state. There was significantly more cell division when any of the four top TFs were perturbed, measured via immunofluorescence of K167, a common cell division marker (FIGS. 5A and 5B) and cell cycle analysis of scRNA-seq data (FIG. 5C). CRA E2F3 and CRA EZH2 caused significant decreases in senescence, assayed through beta-galactosidase staining (Dimri, G. P. et al. A biomarker that identifies senescent human cells in culture and in aging skin in vivo. Proc. Natl. Acad. Sci. U.S.A 92, 9363-9367 (1995)) (FIGS. 6A-6B). While neither CRI ZFX or CRI STAT3 had significantly fewer senescent cells, all these TF perturbations overall had more expression of KI67 mRNA and less expression of senescence associated genes p21, TIMP1, and TIMP2 (Basisty, N. et al. A proteomic atlas of senescence-associated secretomes for aging biomarker development. PLoS Biol. 18, e3000599 (2020))(FIG. 7A-7C). Targeting any one of these TFs in late passage cells led to cell division and senescence markers similar to middle passage cells (WT PD 22), about 10 PDs fewer than the cells' actual PD.

### TF Perturbations Improved Proteostasis Via Increased Proteasome Activity and Fewer Senescence-Associated Lysosomes

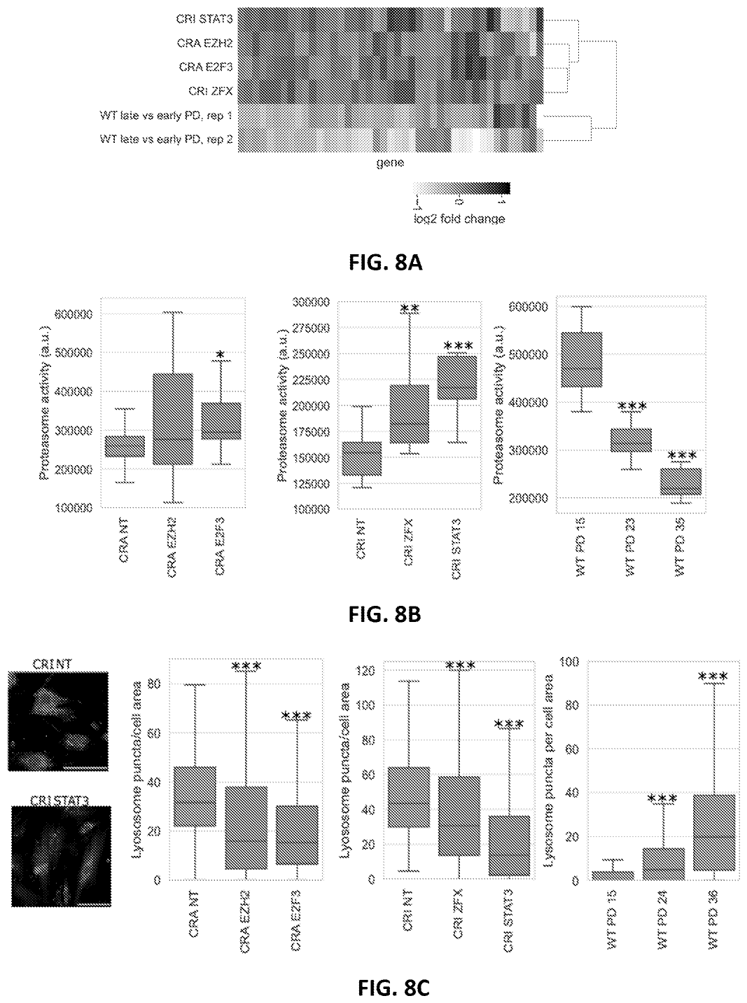

Loss of proteostasis can be a contributor to several diseases and aging (Labbadia, J. & Morimoto, R. I. The biology of proteostasis in aging and disease. Annu. Rev. Biochem. 84, 435-464 (2015)). All these TF perturbations significantly increased proteasome expression, reversing the pattern seen in late passage cells (FIG. 8A). Three of these TF perturbations had significantly more proteasome activity, as measured through a fluorescence-based cleavage assay, while later passage cells had significantly less proteasome activity (FIG. 8B).

Lysosomes can also play a role in proteostasis. Interestingly, there were significant decreases in lysosome puncta per cell area in all TF perturbations, while later passage cells had significantly more lysosome puncta per cell area, as measured by the Lysotracker staining (FIGS. 8C and 8D). An early electron microscopy study found the lysosomes of serially propagated human fibroblasts gradually transform to residual bodies (containing undigested materials), and these bodies increase in number and size (Robbins, E., Levine, E. M. & Eagle, H. Morphologic changes accompanying senescence of cultured human diploid cells. J. Exp. Med. 131, 1211-1222 (1970)), reflecting degeneration of lysosomal function (Gorgoulis, V. et al. Cellular Senescence: Defining a Path Forward. Cell 179, 813-827 (2019)). It is likely the decrease of the number of puncta by the TF perturbations indicated improved lysosomal function. Overall, each of these TF perturbations significantly rejuvenated proteostasis in these late passage fibroblasts.

Proteasome activity was measured using a fluorescence-based assay (Proteasome-Glo™ Assays), where the relative fluorescence of the cleaved substrate correlates with the proteasome activity of the cells. Cells were plated in a dish and the substrate was added to the cells. The cells' proteasomes cleave the substrate which then emits light. The amount of fluorescence is proportional to proteasome activity.

### TF Perturbations Increased Mitochondrial Gene Expression, Metabolism Gene Expression, and Mitochondrial Membrane Potential

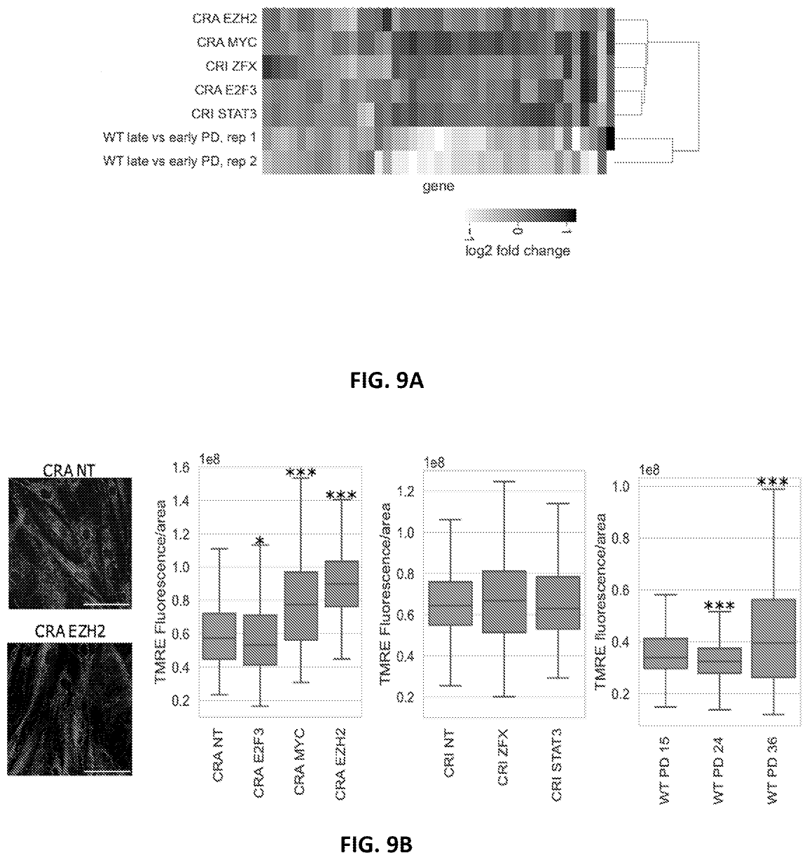

Mitochondria are less functional and mitochondrial genes are less expressed in old cells and late passage cells (López-Otin, C., Blasco, M. A., Partridge, L., Serrano, M. & Kroemer, G. The hallmarks of aging. Cell 153, 1194-1217 (2013); Tigges, J. et al. The hallmarks of fibroblast ageing. Mechanisms of Ageing and Development vol. 138 26-44 (2014)). In all four of the top TF perturbations, there were significant increases in mitochondrial ribosomal genes and metabolism related genes, while WT late passage cells had significantly less of both (FIGS. 9A-B). To assay mitochondrial membrane potential, cells were stained with TMRE (tetramethylrhodamine, ethyl ester) mitochondrial membrane potential marker and measured fluorescence per cell area; more fluorescence means more mitochondrial membrane potential. CRA EZH2 had a significant increase in mitochondrial membrane potential. CRA MYC was used as a positive control for increased mitochondrial membrane potential, given its known roles in mitobiogenesis (Morrish, F. & Hockenbery, D. MYC and mitochondrial biogenesis. Cold Spring Harb. Perspect. Med. 4, (2014)). CRA E2F3 had very slightly, but significantly, less mitochondrial membrane potential. The other TFs did not have significant changes in mitochondrial membrane potential. Overall, all four of the top TFs increased mitochondrial related expression, whether in mitochondrial ribosome gene expression, metabolism gene expression, or membrane potential.

### TF Perturbations Caused Minimal Changes to γH2AX, a DNA Damage Marker

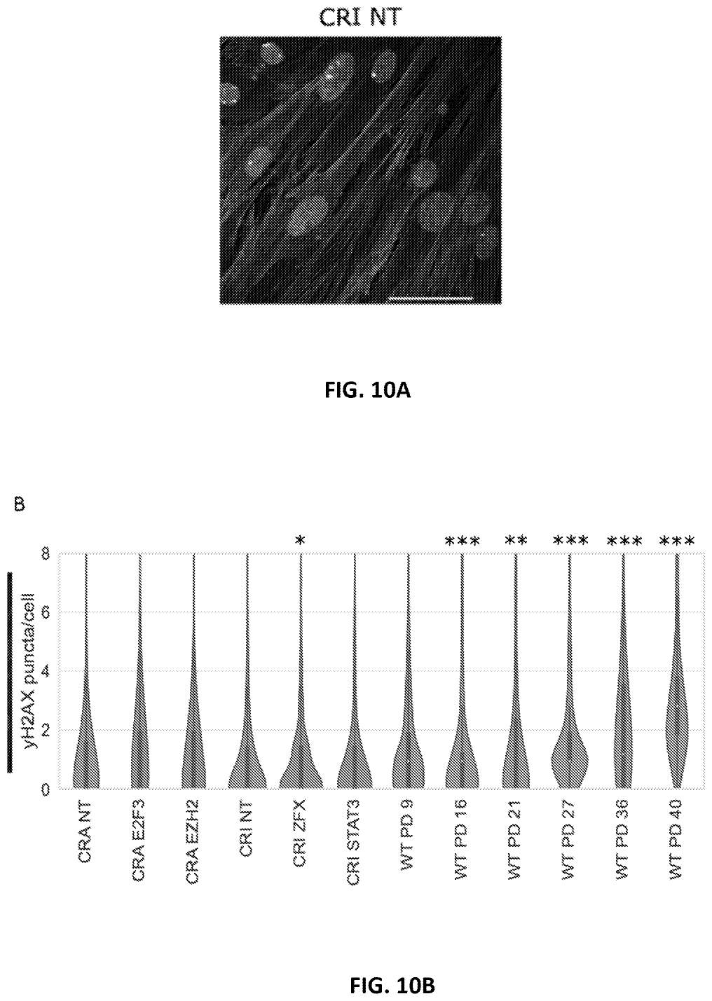

DNA damage increases as cells age, but also as cells grown in culture divide more rapidly (Gruel, G. et al. Cell to Cell Variability of Radiation-Induced Foci: Relation between Observed Damage and Energy Deposition. PLoS One 11, e0145786 (2016); Zorin, V. et al. Spontaneous γH2AX foci in human dermal fibroblasts in relation to proliferation activity and aging. Aging 11, 4536-4546 (2019)). A common way to measure DNA damage is through immunofluorescence imaging of γH2AX, a histone phosphorylation marker adjacent to double stranded DNA breaks. In rejuvenation studies with the Yamanaka factors, γH2AX foci decreased slightly (Ocampo, A. et al. In Vivo Amelioration of Age-Associated Hallmarks by Partial Reprogramming. Cell vol. 167 1719-1733.e12 (2016)). In the top TF perturbations, either no change or a slight increase was observed in γH2AX levels (puncta/cell). In WT passaged cells, γH2AX puncta did increase slightly, but significantly, in later passage cells (FIGS. 10A-B). Overall, γH2AX changes in TF perturbations and WT passaged cells were subtle and may not be the main driving forces in cell aging or rejuvenation phenotypes.

TF Perturbations' Rejuvenation Phenotypes were Independent of Epigenetic Changes in Histone Markers and the Methylation Clock

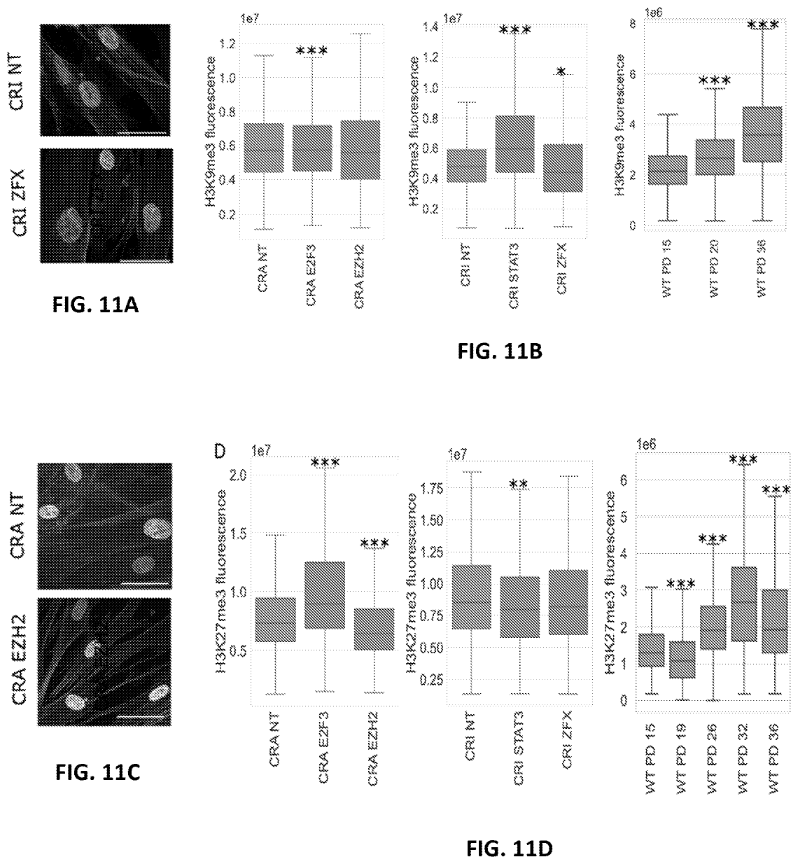

Epigenetic patterns, such as the amount and location of histone and DNA methylation, can regulate gene expression and can have links to aging and senescence (López-Otín, C., Blasco, M. A., Partridge, L., Serrano, M. & Kroemer, G. The hallmarks of aging. Cell 153, 1194-1217 (2013)). The global level of histone 3 lysine 9 trimethylation (H3K9me3) can be used as markers of aging. In the experiments performed, CRI STAT3 had significantly more global H3K9me3, and CRA EZH2 and CRI ZFX had significantly less. In WT passaged fibroblasts, late passage cells had slightly, but significantly more H3K9me3 (FIGS. 11A-D).

Histone 3 lysine 27 trimethylation (H3K27me3) can also be linked to aging, but there are few links between global levels and aging; most research links changes to specific regions of DNA and not to global levels. In some models of aging, more global H3K27me3 was beneficial, and in some models, it was detrimental (Ma, Z. et al. Epigenetic drift of H3K27me3 in aging links glycolysis to healthy longevity in Drosophila. Elife 7, (2018); Maures, T. J., Greer, E. L., Hauswirth, A. G. & Brunet, A. The H3K27 demethylase UTX-1 regulates C. elegans lifespan in a germline-independent, insulin-dependent manner. Aging Cell 10, 980-990 (2011)). As shown here, the TF perturbations had different results; CRA E2F3 had more global H3K27me3 levels and CRA EZH2 and CRI STAT3 had less global H3K27me3 levels. In WT passaged fibroblasts, there was significantly more H3K9me3 and H3K27me3 fluorescence in later passages (FIGS. 11A-D).

It was surprising that CRA EZH2 expression had less global H3K27me3 levels. EZH2 is known to regulate H3K27me3 through the polycomb repressive complex 2 (PRC2) (Viré, E. et al. The Polycomb group protein EZH2 directly controls DNA methylation. Nature 439, 871-874 (2006)., and usually is thought to increase H3K27 methylation. No other elements of the PRC2 complex or H3K27 demethylases changed expression when we overexpress EZH2 was overexpressed (data not shown). Without being bound by any particular theory, more EZH2 expression may lead to more H3K27me3 in certain areas of DNA—like senescence linked genes—but the global levels of H3K27me3 in fact decreases.

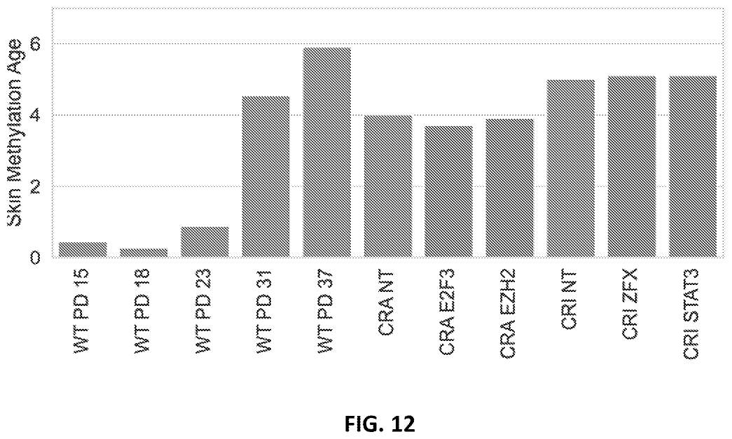

The DNA methylation clock is an analysis that correlates the age of organisms, or population doubling of cells, with patterns of methylation at certain CpG islands. The methylation patterns (or methylation “age”) can determine how old a cell or organism actually is. If cells have a methylation age below the expected value for that age, these cells are deemed “younger” than expected. For example, turning differentiated cells back into stem cells decreases the cells' methylation age back to near zero (Horvath, S. DNA methylation age of human tissues and cell types. Genome Biol. 14, R115 (2013)), indicating these cells returned to their “youngest” state. Experiments were performed to determine whether the TF perturbations caused changes to their methylation age. Specifically, a skin DNA methylation clock was analyzed (Horvath, S. et al. Epigenetic clock for skin and blood cells applied to Hutchinson Gilford Progeria Syndrome and ex vivo studies. Aging 10, 1758-1775 (2018)), because this clock's data were based on skin cells. Methylation clock calculations were performed on passaged WT cells and the top TF perturbations (FIG. 12). The later passage WT cells had progressively “older” methylation clock ages. However, none of the TF perturbations led to changes in the methylation age of the cells. Overall, the rejuvenation phenotypes seen in the TF perturbations neither caused, nor were caused by, changes in the DNA methylation clock.

TF Perturbations' Rejuvenation Phenotypes were Independent of Telomere Length

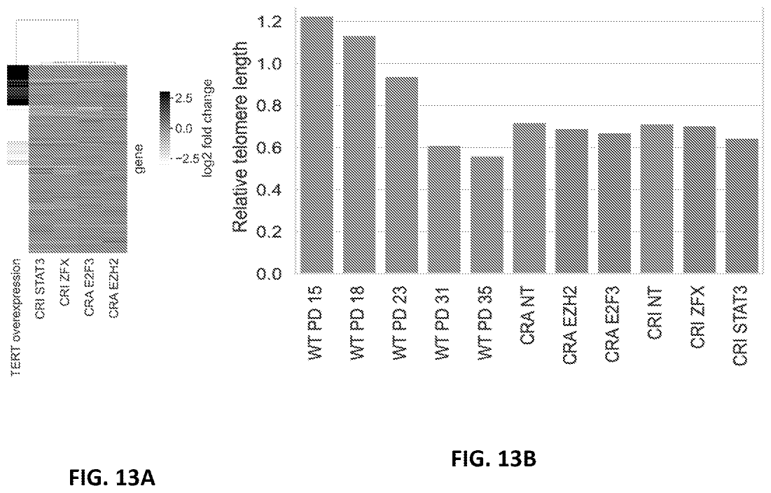

Telomeres, protective caps at the ends of chromosomes, get progressively shorter every time a cell divides. Late passage cells can have shorter telomeres than early passage cells (Harley, C. B., Futcher, A. B. & Greider, C. W. Telomeres shorten during ageing of human fibroblasts. Nature 345, 458-460 (1990)). Overexpressing telomerase (TERT), the enzyme mainly responsible for lengthening telomeres, allows cells to divide indefinitely, regardless of their usual Hayflick limit^{53,54}. Since overexpressing TERT and having longer telomeres can reverse cell aging phenotypes, experiments were performed to determine whether perturbing these TFs caused changes in TERT-related genes and telomere length. Gene expression was compared for the top TF perturbations to previously published data on dermal skin fibroblasts overexpressing TERT (Danielsson, F. et al. Majority of differentially expressed genes are down-regulated during malignant transformation in a four-stage model. Proc. Natl. Acad. Sci. U.S.A 110, 6853-6858 (2013)). None of the TF perturbations have similar gene expression changes to TERT overexpressing fibroblasts (FIG. 13A). Furthermore, TERT mRNA itself was never expressed enough to be measured in the inventors' scRNA-seq experiments (data not shown). The relative length of telomeres was assayed in the TF perturbations and passaged WT cells through qPCR analysis (Lin, J., Smith, D. L., Esteves, K. & Drury, S. Telomere length measurement by qPCR—Summary of critical factors and recommendations for assay design. Psychoneuroendocrinology 99, 271-278 (2019)). A progressive decrease was observed in telomere length in later passage cells, but no change among TF perturbations and their NT control (FIG. 13B). Therefore, the rejuvenation phenotypes seen in the TF perturbations neither caused, nor were caused by, changes in the telomere length.

### TF Perturbations Did not Cause Cancer

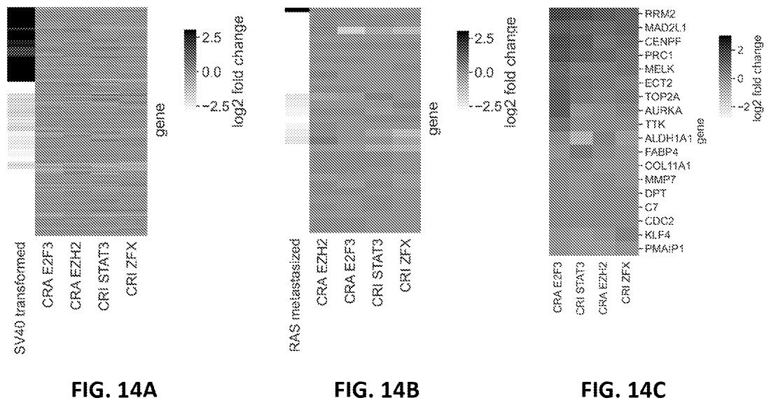

In Yamanaka factor rejuvenation experiments, cells become cancerous if the four TFs are turned on too much or for slightly too long. The top four TF perturbations never caused cells to behave like cancer cells. This behavior remained true, even when perturbed cells were passaged for 2.5 weeks (about 6-9 population doublings); cells maintained their fibroblast identity and did not grow more rapidly than middle passage cells. To further investigate whether the TF perturbed cells behaved like cancer, the gene expression was compared of the TF perturbations to that of dermal skin fibroblasts transformed into cancer cells across several steps; first the cells were immortalized with TERT overexpression, then transformed with SV40 large-T antigen, later metastasized with oncogenic H-Ras (RASG12V) (Danielsson, F. et al. Majority of differentially expressed genes are down-regulated during malignant transformation in a four-stage model. Proc. Natl. Acad. Sci. U.S.A 110, 6853-6858 (2013)). None of the TF perturbations had similar gene expression changes to transformed or metastasized skin fibroblasts (FIGS. 14A-C). Gene expression was also compared in the top TF perturbations to genes commonly changed in seven types of cancer ((Xu, K. et al. A comparative analysis of gene-expression data of multiple cancer types. PLoS One 5, e13696 (2010)). Although some commonly overexpressed cancer genes were observed to be more expressed in the top TF perturbations, since every one of those genes is related to cell cycle and rejuvenated cells have higher expression of cell cycle genes, none of the TF perturbations caused a cancer phenotype in these cells.

Overexpressing the four Yamanaka factors at once is sufficient to cause cancer. While the four top TFs also have links to cancer, changing their expression individually does not seem sufficient to cause cancer. E2F3 is commonly overexpressed in cancer cells; but, overexpressing E2F3 does not seem sufficient to cause cancer (for example, cells need to lose pRB functionality as well). Furthermore, E2F3 was only overexpressed very slightly, less than 0.3 log 2fc (data not shown). EZH2 is commonly overexpressed in cancer but overexpressing it by itself is not sufficient to cause cancer, neither in literature nor in the experiments where EZH2 was overexpressed up to 30-fold more than control cells. Finally, STAT3 and ZFX are overexpressed in cancer, but here, it was observed that repressing these genes causes rejuvenation. These results, and the fact that targeting any one of these TF perturbations rejuvenates cells, demonstrate low cancer risk with the top TF perturbations.

Perturbing any of these TFs can lead to more cell division, healthier proteostasis, more mitochondria expression, and/or less senescence—all classic hallmarks of younger and earlier passage cells. Interestingly, the rejuvenation was independent of classic epigenetic changes and telomere length.

On average, targeting any one of these TFs changes the cell state from around PD 35 back to a state similar to PD 22. In both K167 and senescence counts, perturbed cells had similar rates to WT PD 22 while NT cells remained similar to WT PD 35. Relative gene expression of p21, TIMP1, and TIMP2 also changed to similar levels as of PD 22 cells. Mitochondrial gene expression increased in the TF perturbations, and these genes were significantly less expressed in late passage WT cells. Similarly, relative proteasome activity and expression increased to levels similar to that of middle passage cells. The main differences between these TF perturbations and WT passaged are telomere length and methylation age. In WT cells, late passage cells had shorter telomeres and an older methylation clock age. But, neither telomeres nor the methylation clock changed when the TFs were targeted. The TF perturbations caused transcriptional level rejuvenation across many pathways, and these pathways functioned independently from telomere length and epigenetic changes.

The observation that these four seemingly unrelated TFs caused such similar rejuvenation phenotypes was initially surprising. Although these TFs have different original cellular roles, they seem to be part of a tightly interconnected network, where targeting one node leads to similar downstream cascades and cell phenotypes. For example, EZH2 was significantly more expressed in most TF perturbations with rejuvenating correlation values. Also, whenever ZFX was targeted with either CRI or siRNA, STAT3 also became less expressed (data not shown). The results demonstrate there are multiple starting points that can be targeted in order to ameliorate cellular aging, beyond just the Yamanaka factors. The TF targets presented herein can play a role in rejuvenation research and treatments.

### Example 2: Methods

### Plasmids:

CRISPR related plasmids are pMH0001, pJKNp44, pJR89, pJR85, pMJ114, pMJ117, and pMJI79. To make single sgRNA plasmids, complementary oligonucleotides (Integrated DNA Technologies) encoding the sgRNA protospacer sequence with BstXI/BlpI overhangs were annealed. Second, a double enzyme digest using BstXI/BlpI was performed on either pMJ114, pMJ117, or pMJ179, and the annealed oligonucleotides were ligated to the cut plasmid. In follow-up cell assays, pMJ117 was used, although any of the three sgRNA backbones would yield the same results.

For dual sgRNA production, previous protocols were followed, with the slight modification of changing one digestion enzyme (Replogle, J. M. et al. Combinatorial single-cell CRISPR screens by direct guide RNA capture and targeted sequencing. Nat. Biotechnol. 38, 954-961 (2020)). Briefly, dual-guide libraries were created by PCR amplifying pooled oligonucleotides. These oligonucleotides and pJR85 were digested with BstXI/BlpI and ligated together. Then, this new intermediate plasmid and pJR89 were digested with Esp3I. The resulting piece from pJR89 was ligated into the intermediate pJR85. The final plasmids were validated with NGS sequencing.

Cell Culture, DNA Transfections, Virus Production, CRISPRa and CRISPRi Cell Lines, and sgRNA:

Lenti-X 293T (Lx293T) cells were grown in Dulbecco's modified eagle medium (DMEM) supplemented with 10% FBS and penicillin-streptomycin. Neonatal primary skin fibroblasts were purchased from ATCC and cultured in ATCC's Fibroblast growth kit with low serum, with phenol red and penicillin-streptomycin. These fibroblasts were passaged for almost one year, where they were split about 1:2 or 1:4 at about 80-90% confluency. Different passage stages were frozen (normal medium plus 5% DMSO) for long-term storage. Patient skin fibroblasts were purchased from Coriell Institute, and their standard culturing protocols were followed. Lentivirus was made by transfecting Lx293T with standard packaging vectors and TransIT-LT1 Transfection Reagent (Mirus, MIR 2306). Viral supernatant was harvested two days after transfection, filtered the supernatant through a 0.45 um filter, and either added to cells or frozen in aliquots prior to infection.

Stable cell lines in passaged fibroblasts expressing either CRISPRi (CRI; pMH0001) or CRISPRA (CRA; pJKNp44) were created by transducing either the CRA or CRI vector into early passage fibroblasts, culturing them for a few days, and sorting cells for purity by BFP on the BD FACSAria2. CRA and CRI cell lines were passaged until they were late passage; BFP fluorescence and CRA/CRI activity was maintained across all population doublings. Next, sgRNA vectors were transduced into CRA and CRI cells at the desired population doubling at an MOI of ˜0.3. For all experiments using sgRNA, CRA or CRI cells were infected with the sgRNA vector, recovered for two days, selected for purity using puromycin for 2-3 days (2 ug/mL), recovered for an additional 2 days, and then used for experiments. The selection led to 90-100% purity, as was evident by the bright BFP signal from the sgRNA vectors.

Real-Time Quantitative Polymerase Chain Reaction (qPCR):

The efficacy of sgRNA and gene expression phenotypes were measured through (qPCR). Cells were harvested and total RNA was isolated using the RNeasy Plus Mini Kit (Qiagen, 74134). RNA was converted to eDNA using SuperScript IV (Invitrogen, 18090050) with standard conditions using oligo(dT) primers and RNaseOUT Ribonuclease Inhibitor (Invitrogen, 10777-019). 20 uL qPCR reactions were prepared with 10 μL of the KAPA SYBR FAST Universal MasterMix, 5 uL cDNA (representing 5-20 ng starting RNA per reaction), and 5 μL of forward and reverse primers mixed together 1:1 at 0.8 μM each. Three technical replicates were run for every sample. These reactions were run on the LightCycler 480 (Roche). Quantification of fold change was measured using beta-actin as a control gene.

### Single-Cell RNA Sequencing and Perturb-Seq

For initial WT passaged cell experiments, manufacturer's protocols for the Chromium Single Cell 3′ Kit v2 (10× Genomics) were followed with one change: different WT passage stages were added in a pool and identified with cell membrane barcodes, as explained here⁶⁸. The seRNA-seq library was sequenced on a NovaSeq.

For the CRA/CRI Perturb-seq experiment with dual sgRNA, the top two guides for every TF and non-targeting guides were selected from a previously derived list (Horlbeck, M. A. et al. Compact and highly active next-generation libraries for CRISPR-mediated gene repression and activation. Elife 5, (2016)). Dual direct capture seq was performed as explained previously (Replogle, J. M. et al. Combinatorial single-cell CRISPR screens by direct guide RNA capture and targeted sequencing. Nat. Biotechnol. 38, 954-961 (2020)) to have two sgRNA targeting one TF per cell and then sequence the sgRNA in each cell. Briefly, CRA and CRI cell lines were transduced with two pooled viral libraries (one library for CRA, one for CRI) of 200 dual sgRNA vectors and three dual non-targeting vectors (6 non-targeting guides total). These cells were selected for purity with puromycin, recovered for two days, loaded into the 10× Chromium chip, and processed according to the protocol. The scRNA-seq library was sequenced on a NovaSeq.

### Cellular Rejuvenation Assays:

For cellular rejuvenation assays, single sgRNA vectors were used, using the number one guide per TF.

### Immunofluorescence:

For fluorescent antibody staining with fixed cells (immunofluorescence), on day one cells were plated onto 8 well cell culture treated microscopy slides (ibidi, 80841) so the cells would be at about 70% confluency the next day. On day two, cells were first fixed (4% paraformaldehyde in PBS) for 10 minutes, washed with PBS, and then blocked/permeabilized (2% Bovine Serum Albumin/0.1% Triton X in PBS) for one hour at room temperature. Next, primary antibodies were added (buffer: 0.5% Bovine Serum Albumin/0.1% Triton X in PBS) for one hour at room temperature. The wells were then washed with PBS. Then, secondary antibodies, Hoechst 33342 (Thermo Scientific, 62249) for nuclei staining, and Alexa Fluor™ 546 Phalloidin (Invitrogen, A22283) for actin staining were added to the wells and incubated for one hour at room temperature in the dark. Finally, the wells were washed with PBS and imaged in PBS. Quantification was done using ImageJ.

Live-Cell Imaging with Mitochondria and Lysosome Staining:

For fluorescent staining of mitochondria membrane-potential and lysosomes in live cells, on day one cells were plated onto 8 well cell culture treated microscopy slides (ibidi, 80841) so the cells would be at about 70% confluency the next day. On day two, manufacturer protocols were followed for both TMRE-Mitochondrial membrane potential staining (Abeam, abl 13852) and lysosome LysoTracker™ Red DND-99 staining (Invitrogen, L7528). Quantification was done using ImageJ.

### Proteasome Activity:

Manufacturer protocols were followed for the proteasome activity assay (Proteasome-Glo Chymotrypsin-like cell-based assay, Promega, G8660). Briefly, on day one, cells were plated into white-walled clear bottom cell culture treated 96 well plates. Each well had the same number of cells, between 1-3×10\hat{ }4 cells. On day two, the cell medium was aspirated, and 1:1 cell medium and proteasome-glo reagents were added to each well. The plate was put in a plate shaker for 2 minutes at 700 rpm. The 96 well plate was then incubated at room temperature for a minimum of 10 minutes. The fluorescence was measured on the Promega GloMax plate reader.

### Beta-Galactosidase Staining:

Manufacturer protocols were followed for the Senescence β-Galactosidase Staining (Cell Signaling Technology, 9860). Briefly, on day one cells were plated onto 8 well cell culture treated microscopy slides (ibidi, 80841). On day two, the cells were fixed, the β-Galactosidase stain was added to each well, and the slides were incubated in a 37 C incubator (standard bacterial incubator) overnight. On day three, the stain was removed, cells were washed with PBS, cells were stained with Hoechst to aid in cell counting, and then the slides were imaged. If there is evaporation overnight in the 37 C incubator, salt crystals precipitate out of solution and can change results. To avoid evaporation, the slides were placed in a plastic container with water-soaked paper towels to create a humidity chamber. Quantification was done using ImageJ.

### Relative Telomere Length:

Genomic DNA was extracted from approximately one million cells per condition (QIAamp DNA Blood Mini Kit, 51104). The qPCR telomere length experiments were conducted with the genomic DNA (Lin, J., Smith, D. L., Esteves, K. & Drury, S. Telomere length measurement by qPCR—Summary of critical factors and recommendations for assay design. Psychoneuroendocrinology 99, 271-278 (2019)).

### Methylation Clock:

Genomic DNA was extracted from approximately one million cells per condition (QIAamp DNA Blood Mini Kit, 51104). The genomic DNA was then processed with bisulfite conversion and methylation chip experiments using the Infinium MethylationEPIC Kit. For quantification of the methylation data, the methylclock package was used (Bioinformatic Research Group in Epidemiology (BRGE). methylclock: DNA methylation-based clocks. (Github)), specifically the skinHorvath clock. There were technical replicates for the control samples (CRA NT, CRI NT). Due to a small fraction of CpG islands having inconsistent methylation rates between repeats, these repeats had about 15-30% variability in methylation clock results. To correct for this technical variation, the difference of the repeats was divided by the mean of the repeats, and only those CpGs with less than 15% variability were kept. The more variable CpGs were forced to be zero, meaning they did not contribute to the methylation clock calculations. The specific CpGs set to zero for CRA NT were also set to zero for CRA EZH2 and CRA E2F3; the CpGs set to zero for CRI NT were also set to zero for CRI STAT3 and CRI ZFX. Then, the mean value for technical repeats was found.

### Computational Methods

### Single Cell RNA Sequencing Analysis

CellRanger from 10× Genomics and Scanpy were computational packages used to analyze single cell RNA sequencing data. To find the top rejuvenating TFs, correlation values for gene expression between TF perturbations and early passage cells were calculated as follows. First, all the cells for a TF perturbation or passage stage were grouped together for further gene expression analysis. Then, the log 2 fold change (log 2fc) of every gene for WT early versus late passage fibroblasts was calculated, as was the log 2fc of every gene for a given TF perturbation compared to non-targeting (NT) control cells. The log 2fc of any given gene for WT was plotted on the y-axis, and the log 2fc of the same gene was plotted on the y-axis. The correlation of all these points was found (the log 2fc of a gene in WT passaged cells versus the log 2fc of a gene in a TF perturbation). The TF perturbations with the largest negative r-value had the greatest global gene expression change towards being like early passage cells.

### Statistical Analysis for Cellular Assays:

Experiments were conducted in at least three biological replicates for all sgRNA cell assays. Sometimes repeats of experiments were done on separate days. Because of slight differences in staining efficiencies, the absolute values from different days of experiments varied; for example, while the relative differences between NT and TF perturbations were constant, the absolute values were different. In order to accurately combine data collected from different days, data was normalized through a multiplication constant. The global mean for all the NT (control) samples was found for a given experiment (ex: all the CRA NT values in the mitochondrial membrane potential experiments). Next, the given sub-experiment's mean values were calculated (CRA NT mean values for mitochondrial membrane potential for sub-experiment 1, 2, and 3). A constant was calculated which brought each sub-experiment's NT mean to the global mean. This sub-experiment's constant was multiplied to all the TF perturbations per sub-experiment as well, to normalize those values to the global mean. Then, all the samples were combined.

For continuous data, a Wilcoxon rank-sum test was used to compute p values. For nominal data (KI67 and beta-galactosidase positive rates), binomial distribution was used to compute p values. * p values<0.05, ** p<0.01, and ***p values<0.001.

### Gene Ontology for the Cluster Heatmaps

To create the cluster heatmaps, a collection of sources was used to generate the gene lists. For proteasome and mitochondrial ribosome genes, all human proteasome and mitochondrial ribosome genes were included. For mitochondria and metabolism related genes, all mitochondrial genes (except those encoding tRNA) and the genes derived from the KEGG pathway for “KEGG_CITRATE_CYCLE_TCA_CYCLE”, ID M3985 were included. For the cluster heatmaps on TERT, SV40, and RAS cancer expression, the genes were from Danielsson, F. et al. Majority of differentially expressed genes are down-regulated during malignant transformation in a four-stage model. Proc. Natl. Acad. Sci. U.S.A 110, 6853-6858 (2013). For genes commonly differentially expressed in cancer, the genes were from Xu, K. et al. A comparative analysis of gene-expression data of multiple cancer types. PLoS One 5, e13696 (2010). The log 2 fold change for every gene in each sub-list was found for WT passaged cells and TF perturbations, with no p value cut off. Then, all these fold changes were grouped using their euclidean distances.

### Computational Packages

CellRanger from 10× Genomics and Scanpy were used for single cell RNA sequencing analyses. Bioconductor was used for methylation clock analyses. pySCENIC^{18,19} was used for transcription factor gene regulatory network analyses from single-cell RNA-seq data. ImageJ was used for microscopy analyses. Java TreeView⁷⁰ was utilized for clustering exploration.

TABLE 3
Materials

| | Manufacturer | Concentration/ | |
|---|---|---|---|
| Material name | Identifier | amount used | What used for |
| Agilent 2100 | Agilent | N/A | 10 × library prep |
| Bioanalyzer | N/A | | |
| Agilent High | Agilent | N/A | 10 × library prep |
| Sensitivity DNA Kit | 5067-4626 | | |
| Fibroblast growth kit | ATCC | N/A | Fibroblast cell |
| low serum | PCS-201-041 | | culture |
| Penicillin- | ATCC | 1000× | Fibroblast cell |
| Streptomycin- | PCS-999-002 | | culture |
| Amphotericin B | | | |
| Solution | | | |
| Phenol Red | ATCC | 1000× | Fibroblast cell |
| | PCS-999-001 | | culture |
| Fibroblast Basal | ATCC | N/A | Fibroblast cell |
| Medium | PCS-201-030 | | culture |
| Hemocytometer, | Bulldog Bio | N/A | Cell counting |
| Neubauer Improved | DHC-N01 | | |
| Senescence β- | Cell Signaling Technology | N/A | Senescence staining |
| Galactosidase Staining | 9860 | | |
| Kit | | | |
| Phospho-Histone | Cell Signaling Technology | 500× | antibody staining |
| H2A.X (Ser139) | 9718S | | |
| Rabbit mAb | | | |
| Ki-67 Mouse mAb | Cell Signaling Technology | 10,000× | antibody staining |
| | 9449S | | |
| Tri-Methyl-Histone | Cell Signaling Technology | 2,000× | antibody staining |
| H3 (Lys27) Rabbit | 9733S | | |
| mAb | | | |
| Bovine Serum | Fisher BioReagents | 0.5-2% | antibody staining |
| Albumin | BP9703-100 | | |
| Minimum Essential | Gibco | N/A | Longitudinal |
| Medium | 11095-080 | | fibroblast cell |
| | | | culture |
| Dulbecco's Modified | Gibco | N/A | LX293T and |
| Eagle Medium | 11965-092 | | longitudinal |
| | | | fibroblast cell |
| | | | culture |
| 0.25 % Trypsin-EDTA | Gibco | N/A | Resuspending cells |
| (1×) | 25200-056 | | |
| Trypan Blue Stain | Gibco | 2× | Cell counting |
| (0.4%) | 15250-061 | | |
| DPBS Dulbecco's | Gibco | 1× | General |
| Phosphate Buffered | 14190-144 | | |
| Saline | | | |
| μ-Slide 8 Well | ibidi | N/A | antibody staining |
| chambered slide | 80826 | | |
| Qubit 4 fluorometer | Invitrogen | N/A | DNA concentration |
| | N/A | | |
| Alexa Fluor ™ 488 | Invitrogen | 1 drop per mL | antibody staining |
| donkey anti-rabbit | | antibody | |
| antibody | R37118 | dilution buffer | |
| Qubit dsDNA HS | Invitrogen | N/A | DNA concentration |
| Assay | Q32854 | | |
| Alexa Fluor ™ 488 | Invitrogen | 2,000× | antibody staining |
| donkey anti-mouse | A21202 | | |
| IgG | | | |
| SuperScript IV | Invitrogen | N/A | qPCR |
| Reverse Transcriptase | 18090050 | | |
| RNaseOUT | Invitrogen | 20× | qPCR |
| Ribonuclease Inhibitor | 10777-019 | | |
| Alexa Fluor ™ 546 | Invitrogen | 2,000× | antibody staining |
| Phalloidin | A22283 | | |
| H3K9me3 Polyclonal | Invitrogen | 500× | antibody staining |
| Antibody | PA5-31910 | | |
| XL1-Blue Chemically | Macrolab, UC Berkeley | N/A | Bacterial cloning |
| Competent cells | N/A | | |
| TransIT-LT1 | Mirus | 15 μL per 6-well | transfections |
| transfection reagent | MIR 2304 | of LX293T | |
| | | virus production | |
| Promega GloMax | Promega | N/A | Proteasome |
| plate reader | N/A | | fluorescence assay |
| QIAamp DNA Blood | Qiagen | N/A | gDNA extraction |
| Mini Kit | 51104 | | |
| RNeasy Plus Mini Kit | Qiagen | N/A | RNA extraction |
| | 74134 | | |
| QIAprep Spin | Qiagen | N/A | Plasmid extraction |
| Miniprep Kit | 27106 | | from bacteria |
| LightCycler 480 | Roche | N/A | qPCR |
| Sealing Foil | 04-729-757-001 | | |
| LightCycler 480 | Roche | N/A | qPCR |
| | N/A | | |
| KAPA SYBR FAST | Roche | 2× | qPCR |
| Universal MasterMix | KK4602 | | |
| Triton X-100 | Sigma | 0.1% | antibody staining |
| | X100-100mL | | |
| Polybrene Infection/ | Sigma-Aldrich | 1000× | Infection |
| Transfection Reagent | TR-1003-G | | |
| Puromycin | Sigma-Aldrich | 5000× at 10 | Selection for |
| | 540411 | mg/mL in | infected cells |
| | | DMSO | |
| Dimethyl Sulfoxide | Sigma-Aldrich | N/A | Cell Freezing |
| | D2650 | | |
| NanoDrop One | Thermo Fisher Scientific | N/A | DNA and RNA |
| | N/A | | concentration tests |
| Fetal Bovine Serum | Thermo Fisher Scientific | N/A | Fibroblast cell |
| qualified, USA origin | 26140087 | | culture |
| 10% Formaldehyde | Thermo Scientific | 4% | antibody staining |
| Solution (w/v) | 28906 | | |
| Methanol-free | | | |
| Hoechst 33342 | Thermo Scientific | 10,000× | antibody staining |
| | 62249 | | |
| VWR PCR plate 96 | VWR | N/A | qPCR |
| well low profile half- | 732-3495 | | |
| skirted, LightCycler | | | |
| type | | | |
| LysoTracker ™ Red | Invitrogen | N/A | lysosome staining |
| DND-99 | L7528 | | |
| TMRE-Mitochondrial | Abcam | N/A | mitochondria |
| Membrane Potential | ab113852 | | membrane potential |
| Assay Kit | | | stain |
| pMH0001 UCOE- | Adamson, B. et al., 2016, Cell | N/A | CRISPRI KRAB |
| SFFV-dCas9-BFP- | 167, 1867-1882 | | |
| KRAB | | | |
| pJKNp44 dCas9 VPR; | Tak, Y. 2016, Cell 167, 1867- | N/A | CRISPRA VPR |
| pHR-SFFV-HA-NLS- | 1882 | | |
| dSpCas9(D10A/H840) | | | |
| -VPR-2PA-BFP | | | |
| pJR89 insert for dual | Addgene 140096 | N/A | dual sgRNA |
| sgRNA plasmid | | | |
| pJR85 dual sgRNA | Addgene 140095 | N/A | dual sgRNA |
| plasmid | | | |
| pMJ114 (Plasmid | Addgene 85995 | N/A | sgRNA backbone |
| #85995); sgRNA, | | | |
| bovine promoter | | | |
| pMJ117 (Plasmid | Addgene 85997 | N/A | sgRNA backbone |
| #85997); sgRNA | | | |
| human promoter | | | |
| pMJ179 (Plasmid | Addgene 85996 | N/A | sgRNA backbone |
| #85996) | | | |
| Primary Dermal | ATCC | N/A | cell culture |
| Fibroblast Normal; | PCS-201-010 | | |
| Human, Neonatal | | | |
| (HDFn) | | | |
| Lenti-X ™ 293T Cell | Takara | N/A | virus production |
| Line | 632180 | | |
| Chromium Single Cell | 10× Genomics | N/A | First WT exp and |
| 3′ Kit v2 | 120237 | | longitudinal cells |
| Chromium Next GEM | 10× Genomics | N/A | Perturb-seq and |
| Single Cell 5′ Kit v1 | 1000167 | | second WT exp |
| Protcasome-Glo | Promega | N/A | Proteasome |
| Chymotrypsin-like | G8660 | | fluorescence assay |
| cell-based assay | | | |

TABLE 4
qPCR primers

| | SEQ | | | PrimerBank |
|---|---|---|---|---|
| | ID | | Sequence | ID if |
| Gene | NO: | Direction | (5′-3′) | applicable |
| ACTB | 9 | Forward | AGAGCTACGAGCTG | N/A |
| | | | CCTGAC | |
| | 10 | Reverse | AGCACTGTGTTGGC | |
| | | | GTACAG | |
| CDKN1A | 11 | Forward | TGTCCGTCAGAACC | 310832423c1 |
| (p21) | | | CATGC | |
| | 12 | Reverse | AAAGTCGAAGTTCC | |
| | | | ATCGCTC | |
| EZH2 | 13 | Forward | AATCAGAGTACATG | 322506095c1 |
| | | | CGACTGAGA | |
| | 14 | Reverse | GCTGTATCCTTCGC | |
| | | | TGTTTCC | |
| KI67 | 15 | Forward | ACGCCTGGTTACTA | N/A |
| | | | TCAAAAGG | |
| | 16 | Reverse | CAGACCCATTTACT | |
| | | | TGTGTTGGA | |
| STAT3 | 17 | Forward | CAGCAGCTTGACAC | 47080104c1 |
| | | | ACGGTA | |
| | 18 | Reverse | AAACACCAAAGTGG | |
| | | | CATGTGA | |
| TIMP1 | 19 | Forward | CTTCTGCAATTCCG | 73858576c1 |
| | | | ACCTCGT | |
| | 20 | Reverse | ACGCTGGTATAAGG | |
| | | | TGGTCTG | |
| ZFX | 21 | Forward | GGCAGTCCACAGCA | N/A |
| | | | AGAAC | |
| | 22 | Reverse | TTGGTATCCGAGAA | |
| | | | AGTCAGAAG | |

### Example 3: Rejuvenation of Primary Human Cells

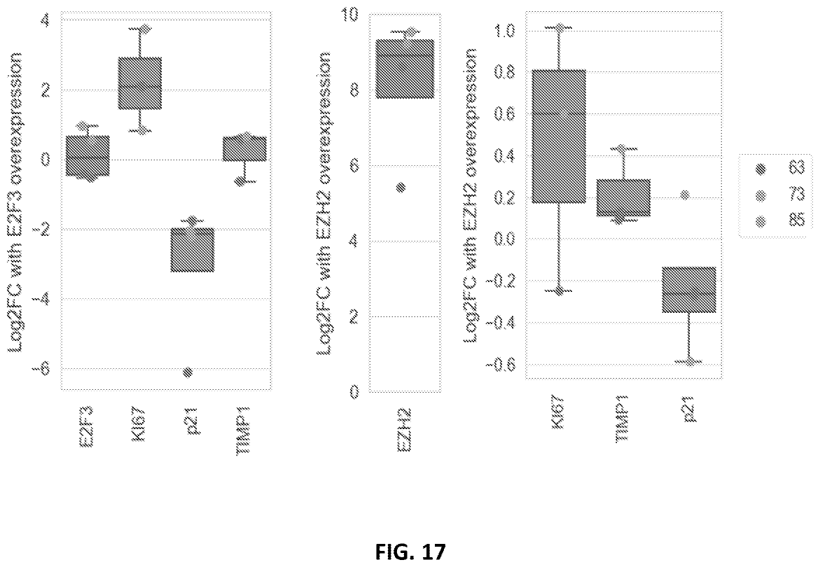

Validation was performed for these TFs in in vivo aged healthy primary patient-derived fibroblasts using the plasmids/siRNA targeting methods. The patients were between 63-89 years old. Overexpression of E2F3 and EZH2 was accomplished with an overexpression plasmid in primary patient-derived fibroblasts. There is generally more KI67 expression—a marker of rejuvenated cells and an indication cells are dividing at a higher rate- and less senescence gene expression as seen in a decrease in p21 expression (FIG. 17).

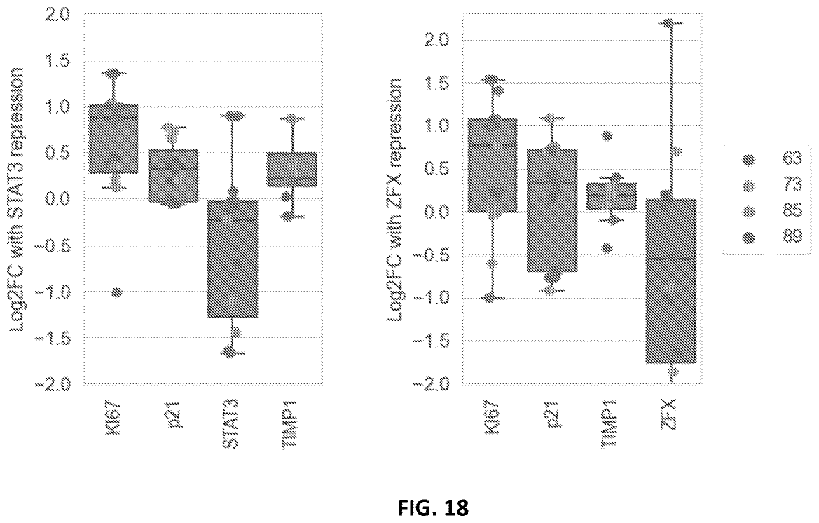

Decrease of STAT3 and ZFX was accomplished with siRNA in primary patient-derived fibroblasts. There is generally more K167 expression and less senescence gene expression (p21, TIMP1) (FIG. 18).

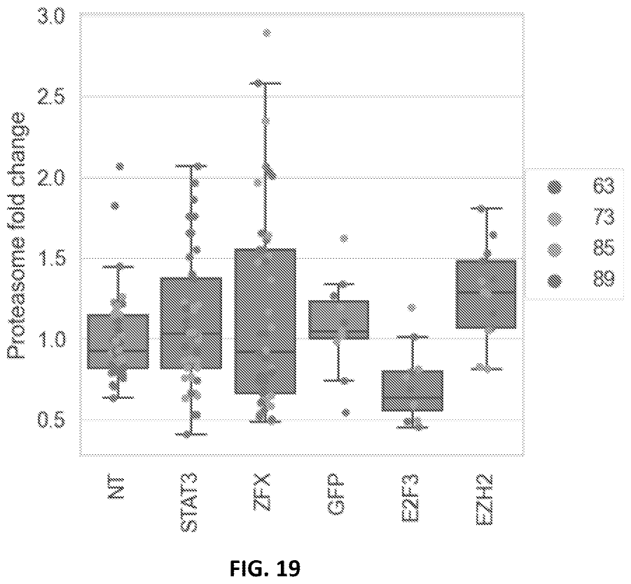

Proteasome activity increased when any of the TFs were targeted (FIG. 19). A higher value means more proteasome activity, which is a sign of cells behaving younger and healthier.

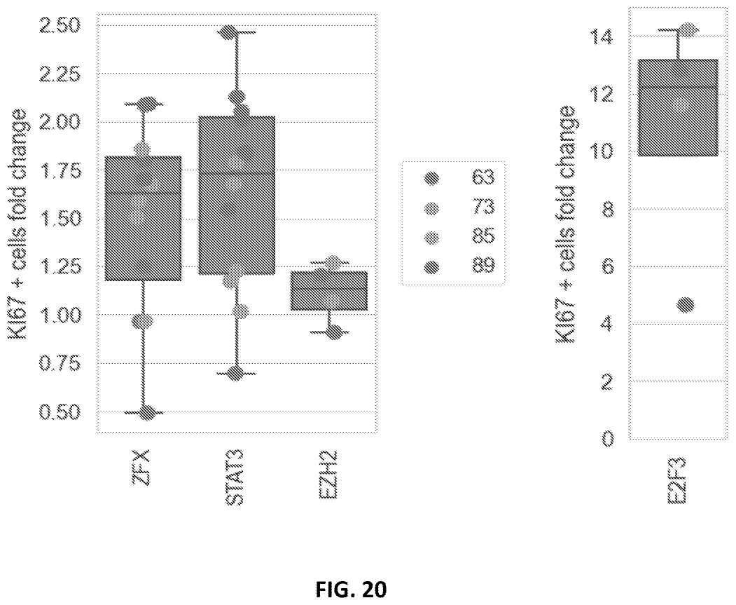

An increase was observed in the percent of cells that are KI67+ when any of the TFs were targeted (FIG. 20). A higher percent of cells that are KI67+ means there are more cells actively dividing.

### Example 4: Rejuvenation of Human Dermal Fibroblasts

Here, it is demonstrated that the method to target the TFs does not need to by CRISPRa or CRISPRi; other methods to target genes, such as the siRNA or overexpression plasmids used here, also lead to rejuvenation phenotypes.

Here, and in the in vivo aged old patient skin cells, an overexpression plasmid delivered via lentivirus was utilized to overexpress either E2F3, EZH2, or GFP as a comparison to CRISPRa. For repression, here and in the in vivo aged old patient skin cells, siRNA was used to target either STAT3, ZFX, or a non-targeting control. The same cell phenotype trends were observed using these orthogonal methods in late passage fibroblasts as observed in late passage CRISPRa and CRISPRi fibroblasts with sgRNA targeting these TFs.

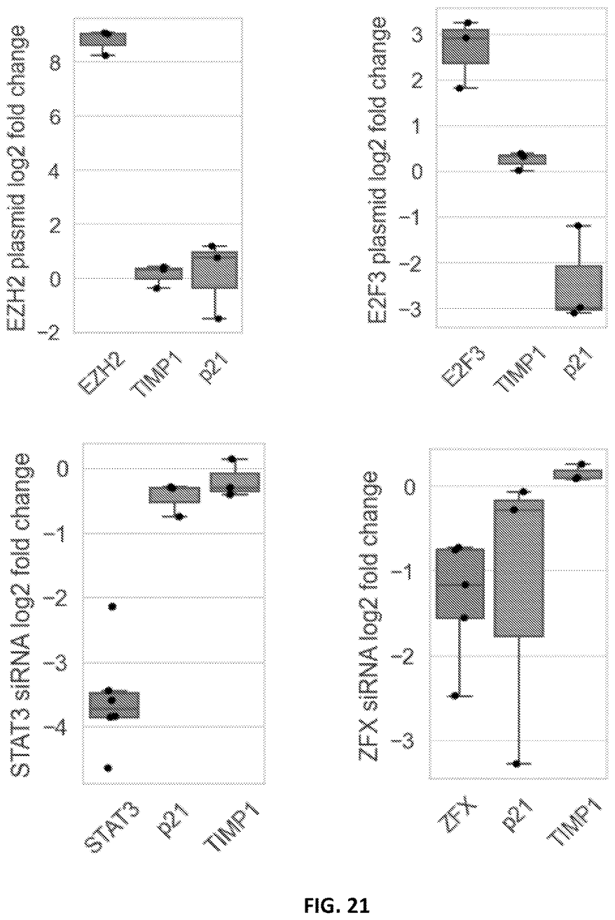

Targeting of TFs of interest resulted in decreased expression of p21, a strong indicator of cellular senescence (FIG. 21).

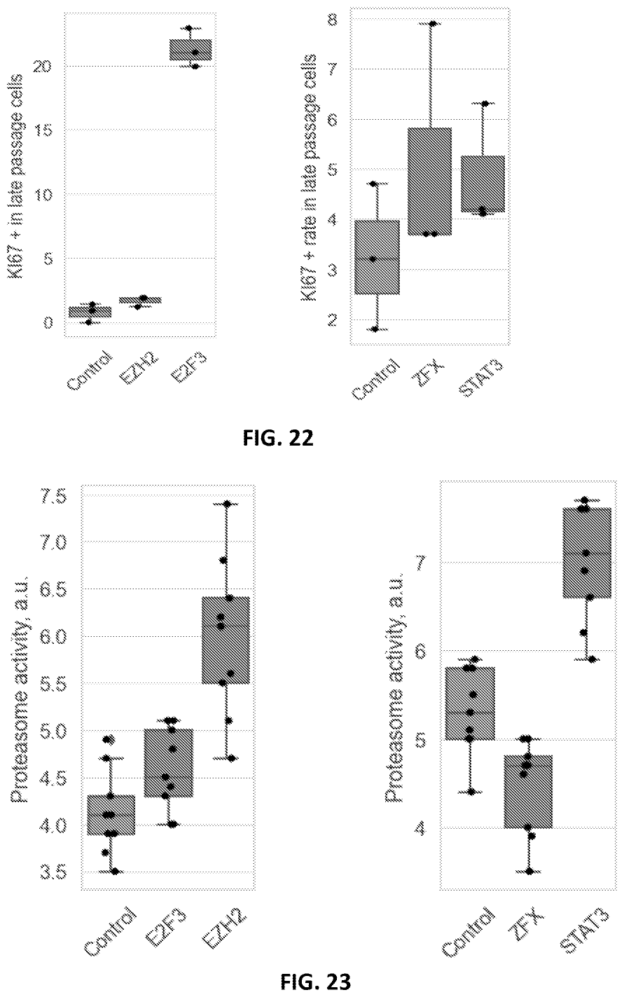

An increase was observed in the percent of cells that are K167+ when any of the TFs are targeted using either siRNA or an overexpression plasmid (FIG. 22). A higher percent of cells that are KI67+ means there are more cells actively dividing. This higher rate is an indication that cells are behaving younger and healthier.

An increase was observed in proteasome activity when targeting three TFs using either siRNA suppression (STAT3) or an overexpression plasmid (E2F3, EZH2) (FIG. 23). Higher proteasome activity is an indication that cells are behaving younger and healthier.

### Example 5: Repressing EGR1 is Rejuvenating

Repressing EGR1 with CRISPRi showed a reversal of global gene expression to a rejuvenated state, as defined by global gene expression being more similar to an earlier passage state (r=−0.55). Further analysis with cell phenotyping showed that repressing EGR1 with CRISPRi also led to several cell aging hallmarks being rejuvenated. They are as follows.

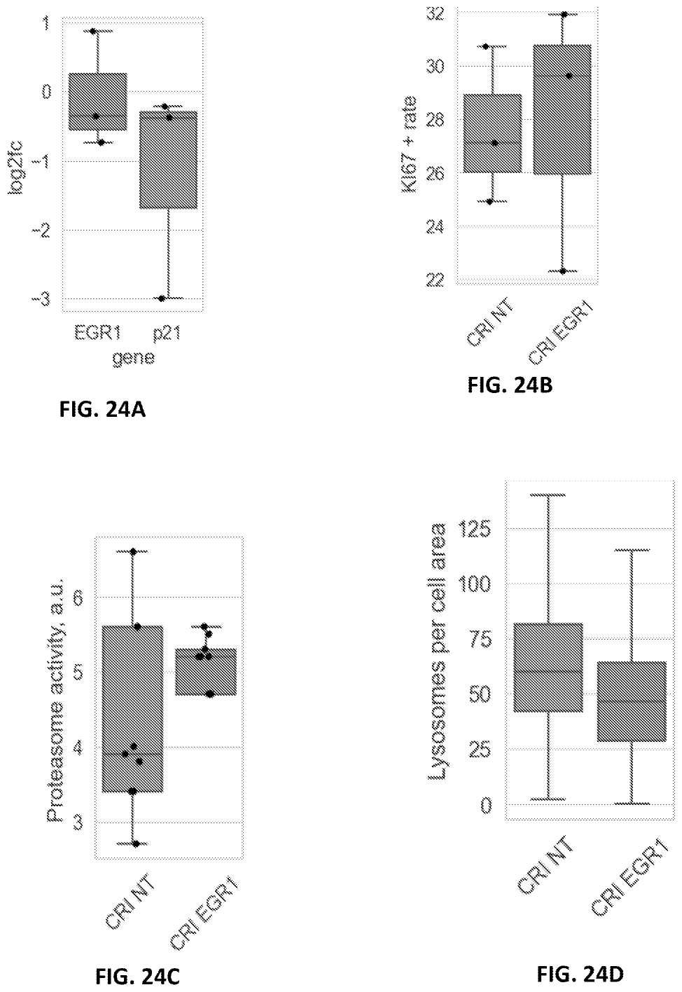

EGR1 is repressed with CRISPRi, as measured by scRNA-seq and by qPCR in two of three biological replicates. Expression of senescence gene p21 was decreased upon repression of EGR1 (FIG. 24A).

The percent of cells that are K167+ increased when EGR1 was repressed with CRISPRi (FIG. 24B). A higher percentage of cells that are K167+ means there are more cells actively dividing. This higher rate is an indication that cells are behaving younger and healthier.

Proteasome activity increased when EGR1 was repressed with CRISPRi (FIG. 24C). Higher proteasome activity is an indication that cells are behaving younger and healthier.

The number of lysosomes per cell area decreased when EGR1 was repressed with CRISPRi (FIG. 24D). Having fewer lysosomes is associated with cells behaving younger and healthier.

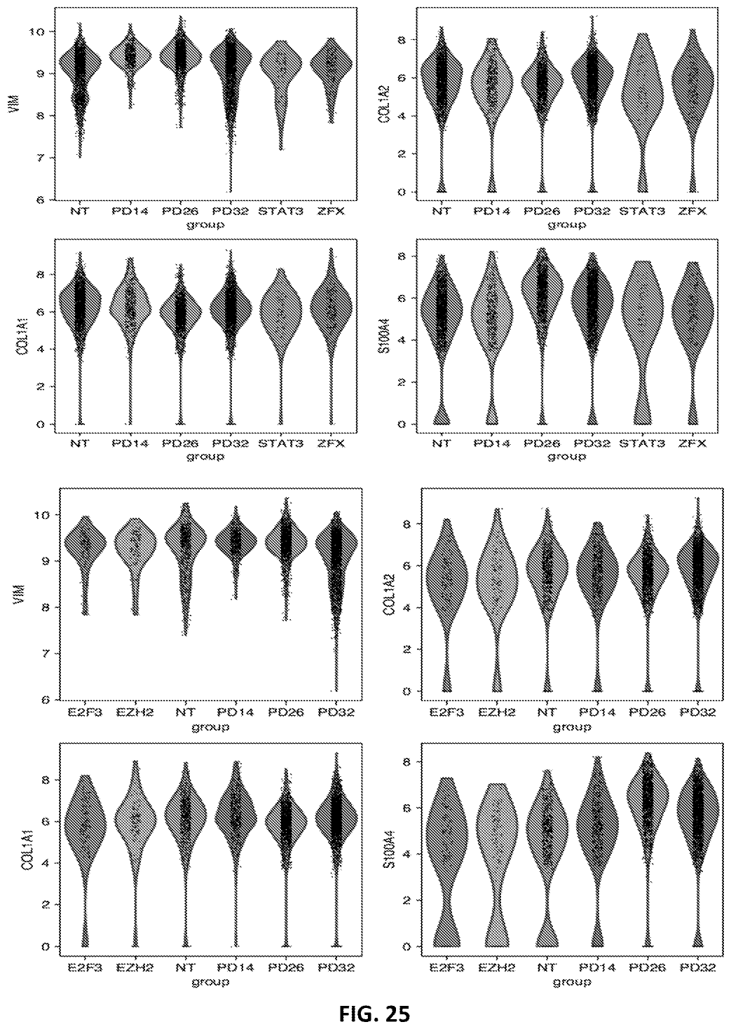

Fibroblast identity genes do not change when TF perturbations are performed. FIG. 25 shows violin plots of common fibroblast marker genes showing their expression is not changed in any of the TF perturbations. Thus, this data and the cell morphology seen in microscopy indicate that cell types are not changed nor causing dedifferentiation with any of the TF perturbations.

### Example 6: Overexpressing FOXM1 is Rejuvenating

Overexpressing FOXM1, done here with CRISPRa, showed a strong reversal of global gene expression to a rejuvenated state, as defined by global gene expression being more similar to an earlier passage state (r=−0.47). Further analysis with cell phenotyping showed that overexpressing FOXM1 with CRISPRa also led to several cell aging hallmarks being rejuvenated. They are as follows.

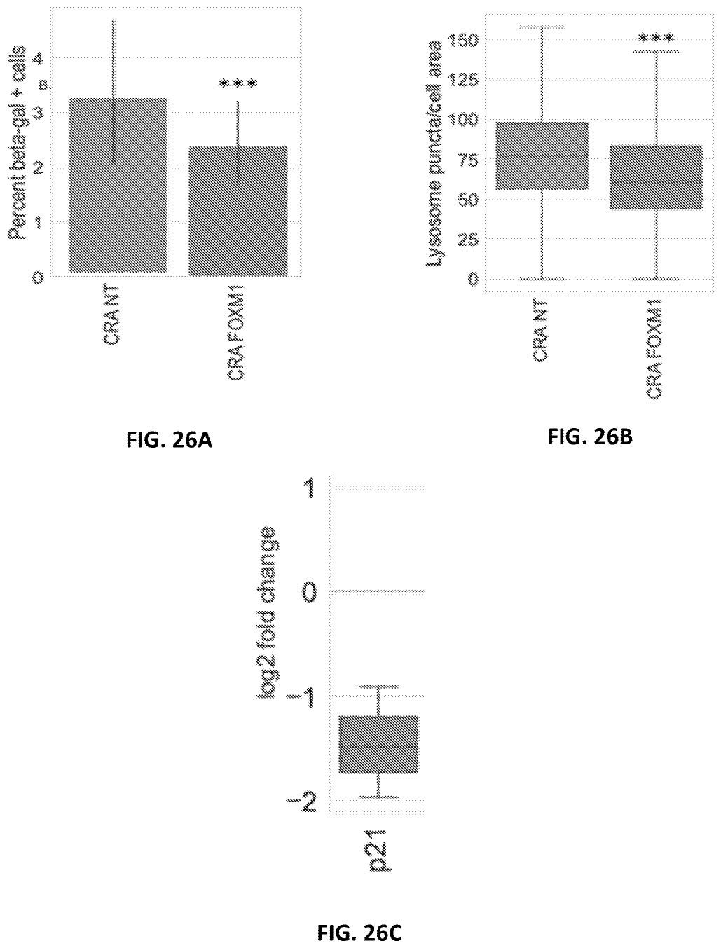

Overexpressing FOXM1 using CRISPRa caused a significant decrease in the number of senescent cells, as measured via the beta-galactosidase assay (FIG. 26A).

Overexpressing FOXM1 using CRISPRa caused there to be significantly fewer lysosome puncta per cell area, as measured with LysoTracker Red (FIG. 26B). Having fewer lysosomes is associated with cells behaving younger and healthier.

Overexpressing FOXM1 using CRISPRa caused there to be about half as much senescence gene expression, as measured by the log 2 fold change in p21 expression (FIG. 26C).

### Example 7: The Rejuvenation Effects Caused by the Overexpression of the Yamanaka Factors (OCT4, KLF4, SOX2, MYC, Commonly Abbreviated OSKM) are Similar to the Rejuvenation Effects Seen when Targeting any of the TFs

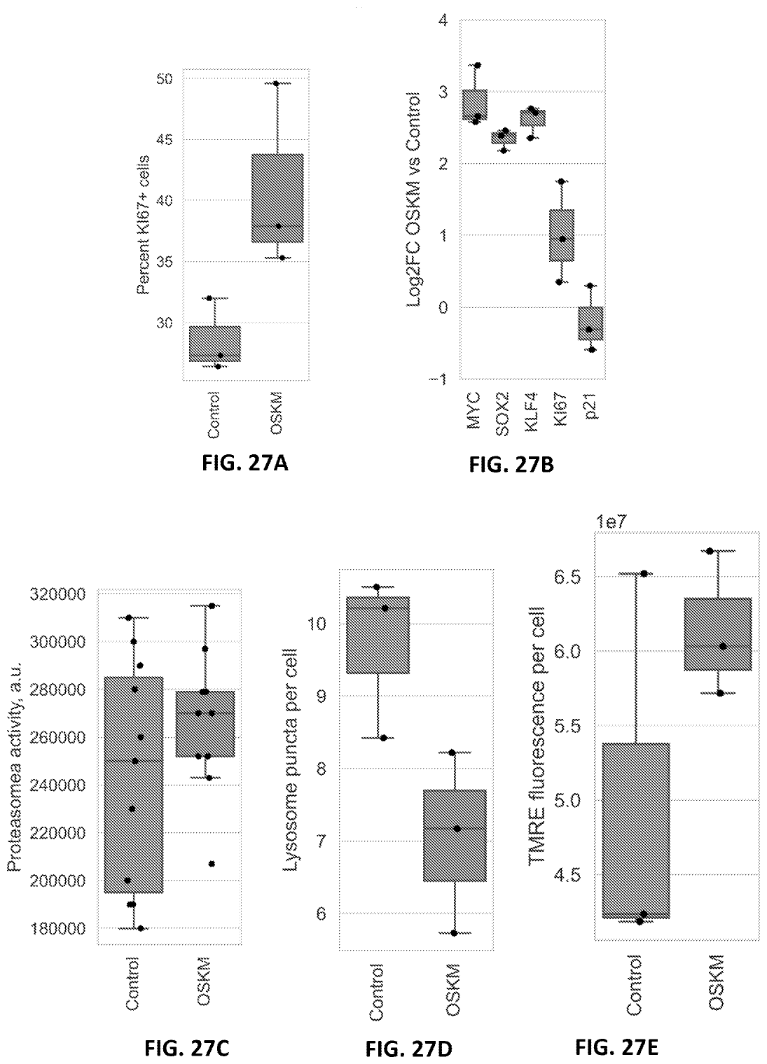

Overexpression of OSKM using an overexpression plasmid led to more K167+cells, meaning more cell division (FIG. 27A).

qPCR demonstrated that the OSKM genes were induced via an overexpression plasmid which then resulted in increased K167 expression and decreased p21 expression (FIG. 27B). Note OCT4 was too lowly expressed in control cells to be accurately measured for comparison here (data not shown).

Overexpression of OSKM using an overexpression plasmid increased proteasome activity (FIG. 27C).

Overexpression of OSKM using an overexpression plasmid led to fewer lysosomes per cell (FIG. 27D).

Overexpression of OSKM led to increased mitochondrial activity, as measured by increased TMRE fluorescence per cell. (FIG. 27E).

### Example 8: Materials and Methods for Examples 3-7

### Overexpression of the Yamanaka Factors

To overexpress the Yamanaka factors, OKSIM (Plasmid #24603 on Addgene) was used. This plasmid overexpresses OSKM constantly and with no need for induction. A control plasmid was used that did not overexpress the Yamanaka factors as comparison (FUW-tetO-hOKMS, Plasmid #51543)). Each plasmid was transfected into late passage WT dermal skin fibroblasts.

Quantitative PCR (qPCR)

Measurement: Rejuvenation was measured with quantitative PCR (qPCR), which measures relative differences in gene expression between samples using the cycle threshold (Ct) values. A control gene's relative expression was measured by a Ct value by performing qPCR using primers specific to that control gene; this control gene is a gene that has constant expression regardless of the cell condition, such as actin a.k.a. ACTB. The particular gene of interest's relative expression was measured by a Ct value by performing qPCR using primers specific to the gene of interest. To find the relative expression of the gene of interest, a comparison was made between its Ct value and that of the control gene. That value was then compared to the values of other genes of interests across other cell conditions. Relative expression of a gene was calculated as the log 2 fold changes (log 2FC). If Log 2FC is +1, that means this gene was two times more expressed in that condition than in the control condition. If the value is −1, that means this gene was half as highly expressed in that condition than in the control condition.

### Proteasome Activity

Measurement: Proteasome activity was measured here using the Promega proteasome activity assay (Cell-Based Proteasome-Glo™ Assays). Here, a luminogenic substrate that fluoresces when it is degraded was added to cells grown in a dish, such as a 96 well dish. The same number of cells was added to each well. When those cells are lysed, their proteasomes are released, and they degrade the luminogenic substrate. Changes in fluorescence were measured using a fluorescence plate reader. An increase in fluorescence means there was higher proteasome activity. Three of these TFs led to increased proteasome activity in this model. Higher proteasome activity is an indication that cells are behaving younger and healthier.

### KI67 Positive Cells by Immunofluorescence Microscopy

Measurement: KI67 positive cells were measured by using immunofluorescence staining. K167 positive cells were quantified by fluorescence microscopy here. Briefly, cells are first plated on a microscope slide, fixed, treated with an antibody specific for KI67, treated with fluorescent antibody to bind the first antibody, and then imaged. A cell is positive if it has a positive fluorescence signal above background noise for the K167 antibody in the nucleus of the cells when staining for K167.

### Lysosomes by Fluorescence Microscopy

Measurement: Total lysosomes in live cells were measured using the Lysotracker Red (LysoTracker™ Red DND-99) fluorescence stains on a fluorescence microscope. Cells were stained, as per the manufacturer's protocol. Lysosomes were quantified by measuring either total puncta, as measured on ImageJ software analysis where maxima are measured, or total fluorescence from lysosomes in an image.

### Example 9: Rejuvenation of Aged Mouse Liver In Vivo Via Overexpression of EZH2

Here, overexpressing EZH2 in the liver of an aged mouse was shown to significantly rejuvenate the liver back to a younger state.

As measured by RNA sequencing of mouse liver, young mice expressed more EZH2 in the liver than old mice. To specifically overexpress EZH2 in mouse livers, AAV8 was used to deliver a standard overexpression plasmid to the liver. The plasmid either encoded for the overexpression of EZH2 or GFP as a control. Both young and aged mice were injected with the AAV8. Thus, there were four treatment groups: aged with EZH2 targeted, aged with GFP targeted, young with EZH2 targeted, and young with GFP targeted. RNA sequencing was then performed on the livers to measure changes in gene expression.

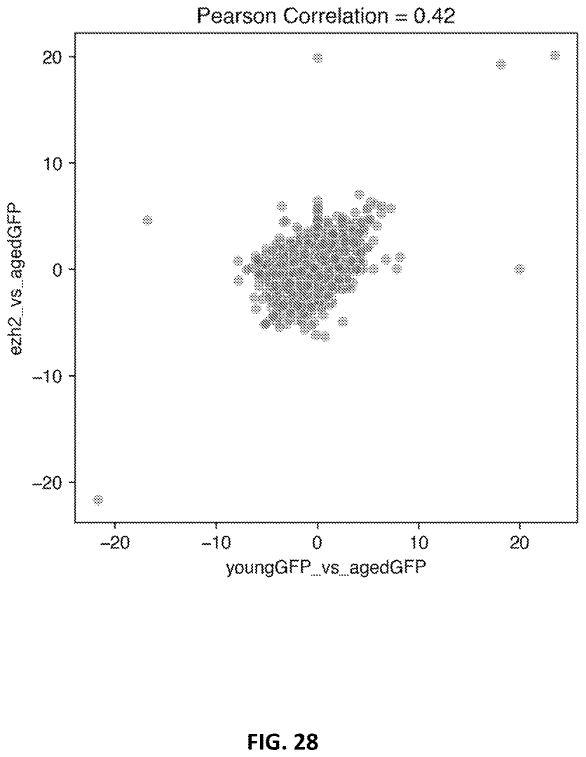

RNA sequencing data showed a significant reversal of gene expression across thousands of genes back to a younger state after the overexpression of EZH2 (FIG. 28). A gene shows reversal back to a younger state if the log 2fc correlate directly with each group (ex: highly expressed in young mice, and highly in aged mice with EZH2 overexpressed). The Pearson correlation of all these genes points was found to be 0.42. Among the most significantly induced genes in the EZH2 overexpression model in aged mice included cytochrome genes, which are known to be critical for metabolism and healthy liver function (McDonnell, J Adv Pract Oncol. 2013, (4): 263-268).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EMBODIMENTS

Embodiment 1. A method for rejuvenating a cell, the method comprising:
(a) increasing the activity of at least one transcription factor selected from:
   (i) E2F Transcription Factor 3 (E2F3); and
   (ii) Enhancer Of Zeste 2 Polycomb Repressive Complex 2 Subunit (EZH2); or
(b) decreasing the activity of at least one transcription factor selected from:
   (i) Signal Transducer And Activator Of Transcription 3 (STAT3); and
   (ii) Zinc Finger Protein X-Linked (ZFX).

Embodiment 2. The method of embodiment 1, wherein the method comprises:
(a) increasing the activity of at least one transcription factor selected from:
   (i) E2F Transcription Factor 3 (E2F3); and
   (ii) Enhancer Of Zeste 2 Polycomb Repressive Complex 2 Subunit (EZH2); and
(b) decreasing the activity of at least one transcription factor selected from:
   (i) Signal Transducer And Activator Of Transcription 3 (STAT3); and
   (ii) Zinc Finger Protein X-Linked (ZFX).

Embodiment 3. The method of embodiment 1 or 2, wherein the level of the activity of the at least one transcription factor is increased or decreased, thereby rejuvenating the cell.

Embodiment 4. The method of any one of embodiments 1-3, wherein the method comprises increasing the activities of E2F3 and EZH2.

Embodiment 5. The method of any one of embodiments 1-3, wherein the method comprises decreasing the activities of STAT3 and ZFX.

Embodiment 6. The method of any one of embodiments 1-3, wherein the method comprises increasing the activity of E2F3 and decreasing the activity of STAT3.

Embodiment 7. The method of any one of embodiments 1-3, wherein the method comprises increasing the activity of E2F3 and decreasing the activity of ZFX.

Embodiment 8. The method of any one of embodiments 1-3, wherein the method comprises increasing the activity of EZH2 and decreasing the activity of STAT3.

Embodiment 9. The method of any one of embodiments 1-3, wherein the method comprises increasing the activities of EZH2 and decreasing the activity of ZFX.

Embodiment 10. The method of any one of embodiments 1-3, wherein the method comprises increasing the activities of E2F3 and EZH2 and decreasing the activity of STAT3.

Embodiment 11. The method of any one of embodiments 1-3, wherein the method comprises increasing the activities of E2F3 and EZH2 and decreasing the activity of ZFX.

Embodiment 12. The method of any one of embodiments 1-3, wherein the method comprises increasing the activity of EZH2 and decreasing the activities of STAT3 and ZFX.

Embodiment 13. The method of any one of embodiments 1-3, wherein the method comprises increasing the activities of E2F3 and EZH2 and decreasing the activities of STAT3 and ZFX.

Embodiment 14. The method of any one of embodiments 1-13, wherein the cell is a human cell.

Embodiment 15. The method of any one of embodiments 1-13, wherein the cell is an adult human cell.

Embodiment 16. The method of any one of embodiments 1-13, wherein the cell is a primary human cell.

Embodiment 17. The method of any one of embodiments 1-13, wherein the cell is a primary adult human cell.

Embodiment 18. The method of any one of embodiments 1-13, wherein the cell is a differentiated cell.

Embodiment 19. The method of any one of embodiments 1-18, wherein the cell is an epithelial cell.

Embodiment 20. The method of any one of embodiments 1-18, wherein the cell is a skin cell, a lung cell, a liver cell, a muscle cell, a pancreatic cell, an immune cell, a bone cell, or a brain cell.

Embodiment 21. The method of any one of embodiments 1-18, wherein the cell is a fibroblast.

Embodiment 22. The method of embodiment 21, wherein the cell is a skin fibroblast.

Embodiment 23. The method of embodiment 21, wherein the cell is a liver fibroblast.

Embodiment 24. The method of any one of embodiments 1-23, wherein the method results in an increase in cell division from the cell as compared to a corresponding untreated cell.

Embodiment 25. The method of embodiment 24, wherein the method results in at least 1%, 2%, 3%, 4%, or 5% increase in cell division.

Embodiment 26. The method of any one of embodiments 1-23, wherein the method results in a cell that is capable of increasing cell division as compared to a corresponding untreated cell.

Embodiment 27. The method of embodiment 26, wherein the cell is capable of increasing cell division by at least 1%, 2%, 3%, 4%, or 5%.

Embodiment 28. The method of any one of embodiments 24-27, wherein the increase in cell division is determined by an increase of the number of KI67 positive cells in a cell population.

Embodiment 29. The method of any one of embodiments 1-28, wherein the method results in a decrease in expression of at least one senescence related gene in the cell as compared to a corresponding untreated cell.

Embodiment 30. The method of embodiment 29, wherein the at least one senescence related gene is selected from the group consisting of p21, TIMP1, and TIMP2.

Embodiment 31. The method of embodiment 29 or 30, wherein the method results in at least 1%, 2%, 3%, 4%, or 5% decrease in expression of the at least one senescence related gene.

Embodiment 32. The method of any one of embodiments 29-31, wherein the decrease in expression of the at least one senescence related gene is determined by an increase of the number of beta-galactosidase-positive cells.

Embodiment 33. The method of any one of embodiments 1-32, wherein the method results in an increase in proteasome activity in the cell as compared to a corresponding untreated cell.

Embodiment 34. The method of embodiment 33, wherein the method results in at least 1%, 2%, 3%, 4%, or 5% increase in the proteasome activity.

Embodiment 35. The method of any one of embodiments 1-32, wherein the method results in a cell that is capable of increasing proteasome activity as compared to a corresponding untreated cell.

Embodiment 36. The method of embodiment 35, wherein the cell is capable of increasing proteasome activity by at least 1%, 2%, 3%, 4%, or 5%.

Embodiment 37. The method of any one of embodiments 33-36, wherein the increased proteasome activity is determined by an increase of proteasome-mediated cleavage of a substrate.

Embodiment 38. The method of embodiment 37, wherein the increased proteasome activity is measured by a fluorescence-based cleavage assay.

Embodiment 39. The method of any one of embodiments 1-38, wherein the method results in a decrease in senescence-associated lysosomes in the cell as compared to a corresponding untreated cell.

Embodiment 40. The method of embodiment 39, wherein the method results in at least 1%, 2%, 3%, 4%, or 5% decrease in the senescence-associated lysosomes.

Embodiment 41. The method of embodiment 39 or 40, wherein the decrease in the senescence-associated lysosomes is determined by a decrease of the number of lysosome puncta per cell area.

Embodiment 42. The method of embodiment 41, wherein the number of lysosome puncta per cell area is measured by Lysotracker™ staining.

Embodiment 43. The method of any one of embodiments 1-42, wherein the method results in an increase in mitochondrial or metabolism gene expression in the cell as compared to a corresponding untreated human adult cell. Exemplary mitochondrial and metabolism genes which may have increased expression include one or more of MT-ATP6, MT-ATP8, MT-CYB, MT-CO1, MT-CO2, MT-CO3, MT-ND4L, MT-ND1, MT-ND2, MT-ND3, MT-ND4, MT-ND5, MT-ND6, ACLY, ACO1, ACO2, CS, DLAT, DLD, DLST, FH, IDH1, IDH2, IDH3A, IDH3B, IDH3G, MDH1, MDH2, OGDH, OGDHL, PC, PCK1, PCK2, PDHA1, PDHA2, PDHB, SDHA, SDHB, SDHC, SDHD, SUCLA2, SUCLG1, and SUCLG2.

Embodiment 44. The method of any one of embodiments 1-43, wherein the method results in an increase in mitochondrial and metabolism gene expression in the cell as compared to a corresponding untreated cell. Exemplary mitochondrial and metabolism genes which may have increased expression include one or more of MT-ATP6, MT-ATP8, MT-CYB, MT-CO1, MT-CO2, MT-CO3, MT-ND4L, MT-ND1, MT-ND2, MT-ND3, MT-ND4, MT-ND5, MT-ND6, ACLY, ACO1, ACO2, CS, DLAT, DLD, DLST, FH, IDH1, IDH2, IDH3A, IDH3B, IDH3G, MDH1, MDH2, OGDH, OGDHL, PC, PCK1, PCK2, PDHA1, PDHA2, PDHB, SDHA, SDHB, SDHC, SDHD, SUCLA2, SUCLG1, and SUCLG2.

Embodiment 45. The method of any one of embodiments 1-44, wherein the method results in an increase in mitochondrial membrane potential in the cell as compared to a corresponding untreated cell.

Embodiment 46. The method of embodiment 45, wherein the increase in mitochondrial membrane potential is measured by TMRE (tetramethylrhodamine, ethyl ester) membrane potential staining.

Embodiment 47. The method of embodiment 1, wherein the method further comprises:
(a) increasing the activity of at least one transcription factor selected from:
   (i) Homeobox protein DLX-6 (DLX6);
   (ii) Forkhead Box M1 (FOXM1);
   (iii) FOS Like 1, AP-1 Transcription Factor Subunit (FOSL1);
   (iv) Nuclear Factor Of Activated T Cells 4 (NFATC4);
   (v) Myc proto-oncogene protein (MYC);
   (vi) Signal Transducer And Activator Of Transcription 4 (STAT4);
   (vii) GATA Binding Protein 3 (GATA3);
   (viii) Heat Shock Transcription Factor 2 (HSF2);
   (ix) Paired Box 4 (PAX4);
   (x) NK2 Homeobox 2 (NKX2-2);
   (xi) SIM BHLH Transcription Factor 2 (SIM2);
or
(b) decreasing the activity of at least one transcription factor selected from:
   (i) Early growth response protein 1 (EGR1);
   (ii) Activating Transcription Factor 2 (ATF2);
   (iii) Vascular Endothelial Zinc Finger 1 (VEZF1);
   (iv) MYC Associated Zinc Finger Protein (MAZ);
   (v) SRY-Box Transcription Factor 2 (SOX2);
   (vi) Paired Box 8 (PAX8);
   (vii) Zinc Finger Homeobox 3 (ZFHX3);
   (viii) Autophagy Related 4C Cysteine Peptidase (ATG4C);
   (ix) Autophagy Related 5 (ATG5);
   (x) High Mobility Group Box 1 (HMGB1);
   (xi) GATA Binding Protein 2 (GATA2); and
   (xii) KLF Transcription Factor 4 (KLF4).

Embodiment 48. The method of any one of embodiments 1-47, wherein the method comprises increasing the activity of E2F3 in the cell.

Embodiment 49. The method of embodiment 48, wherein increasing the activity of E2F3 comprises increasing the mRNA level of E2F3 in the cell.

Embodiment 50. The method of embodiment 49, wherein the mRNA level of E2F3 is increased by at least 1%, 2%, 3%, 4%, 5%, or 10% as compared to a corresponding untreated cell.

Embodiment 51. The method of embodiment 47, wherein increasing the activity of E2F3 comprising increasing the protein level of E2F3 in the cell.

Embodiment 52. The method of embodiment 51, wherein the protein E2F3 is increased by at least 1%, 2%, 3%, 4%, 5%, or 10% as compared to a corresponding untreated cell.

Embodiment 53. The method of any one of embodiments 1-47, wherein the method comprises increasing the activity of EZH2 in the cell.

Embodiment 54. The method of embodiment 53, wherein increasing the activity of EZH2 comprises increasing the mRNA level of EZH2 in the cell.

Embodiment 55. The method of embodiment 54, wherein the mRNA level of EZH2 is increased by at least 1%, 2%, 3%, 4%, 5%, or 10% as compared to a corresponding untreated cell.

Embodiment 56. The method of embodiment 53, wherein increasing the activity of EZH2 comprises increasing the protein level of EZH2 in the cell.

Embodiment 57. The method of embodiment 56, wherein the protein level of EZH2 is increased by at least 1%, 2%, 3%, 4%, 5%, or 10% as compared to a corresponding untreated cell.

Embodiment 58. The method of embodiment 47, wherein the method comprises increasing the activity of DLX6 in the cell.

Embodiment 59. The method of embodiment 58, wherein increasing the activity of DLX6 comprises increasing the mRNA level of DLX6 in the cell.

Embodiment 60. The method of embodiment 59, wherein the mRNA level of DLX6 is increased by at least 1%, 2%, 3%, 4%, 5%, or 10% as compared to a corresponding untreated cell. Embodiment 61. The method of embodiment 58, wherein increasing the activity of DLX6 comprises increasing the protein level of DLX6 in the cell.

Embodiment 62. The method of embodiment 61, wherein the mRNA level of DLX6 is increased by at least 1%, 2%, 3%, 4%, 5%, or 10% as compared to a corresponding untreated cell. Embodiment 63. The method of embodiment 47, wherein the method comprises increasing the activity of FOXM1 in the cell.

Embodiment 64. The method of embodiment 63, wherein increasing the activity of FOXM1 comprises increasing the mRNA level of FOXM1 in the cell.

Embodiment 65. The method of embodiment 64, wherein the mRNA level of FOXM1 is increased by at least 1%, 2%, 3%, 4%, 5%, or 10% as compared to a corresponding untreated cell.

Embodiment 66. The method of embodiment 63, wherein increasing the activity of FOXM1 comprising increasing the protein level of FOXM1 in the cell.

Embodiment 67. The method of embodiment 66, wherein the protein FOXM1 is increased by at least 1%, 2%, 3%, 4%, 5%, or 10% as compared to a corresponding untreated cell.

Embodiment 68. The method of any one of embodiments 1-67, wherein increasing the activity of the at least one transcription factor comprises introducing into the cell one or more polynucleic acids encoding the at least one transcription factor, thereby increasing transcription from the at least one transcription factor.

Embodiment 69. The method of any one of embodiments 47-52, wherein increasing the activity of E2F3 comprises introducing into the cell one or more polynucleic acids encoding E2F3.

Embodiment 70. The method of any one of embodiments 53-57, wherein increasing the activity of EZH2 comprises introducing into the cell one or more polynucleic acids encoding EZH2.

Embodiment 71. The method of any one of embodiments 58-62, wherein increasing the activity of DLX6 comprises introducing into the cell one or more polynucleic acids encoding DLX6.

Embodiment 72. The method of any one of embodiments 63-67, wherein increasing the activity of FOXM1 comprises introducing into the cell one or more polynucleic acids encoding FOXM1.

Embodiment 73. The method of embodiment 68, wherein the method comprising introducing into the cell (i) one or more polynucleic acids encoding E2F3, and (ii) one or more polynucleic acids encoding EZH2.

Embodiment 74. The method of embodiment 73, wherein the method further comprises introducing into the cell one or more polynucleic acids encoding DLX6 or one or more polynucleic acids encoding FOXM1.

Embodiment 75. The method of any one of embodiments 68-74, wherein the one or more polynucleic acids are comprised in a viral vector.

Embodiment 76. The method of any one of embodiments 68-74, wherein the one or more polynucleic acids are comprised in a non-viral vector.

Embodiment 77. The method of any one of embodiments 68-74, wherein the one or more polynucleic acids are encapsulated in a lipid nanoparticle (LNP).

Embodiment 78. The method of any one of embodiments 1-46, wherein the method comprises decreasing the activity of STAT3 in the cell.

Embodiment 79. The method of embodiment 78, wherein decreasing the activity of STAT3 comprises decreasing the mRNA level of STAT3 in the cell.

Embodiment 80. The method of embodiment 79, wherein the mRNA level of STAT3 is decreased by at least 1%, 2%, 3%, 4%, 5%, or 10% as compared to a corresponding untreated cell. Embodiment 81. The method of embodiment 78, wherein decreasing the activity of STAT3 comprises decreasing the protein level of STAT3 in the cell.

Embodiment 82. The method of embodiment 81, wherein the protein level of STAT3 is decreased by at least 1%, 2%, 3%, 4%, 5%, or 10% as compared to a corresponding untreated cell.

Embodiment 83. The method of any one of embodiments 1-46, wherein the method comprises decreasing the activity of ZFX in the cell.

Embodiment 84. The method of embodiment 83, wherein decreasing the activity of ZFX comprises decreasing the mRNA level of ZFX in the cell.

Embodiment 85. The method of embodiment 84, wherein the mRNA level of ZFX is decreased by at least 1%, 2%, 3%, 4%, 5%, or 10% as compared to a corresponding untreated cell.

Embodiment 86. The method of embodiment 83, wherein decreasing the activity of ZFX comprises decreasing the protein level of ZFX in the cell.

Embodiment 87. The method of embodiment 86, wherein the protein level of ZFX is decreased by at least 1%, 2%, 3%, 4%, 5%, or 10% as compared to a corresponding untreated cell.

Embodiment 88. The method of embodiment 47, wherein the method comprises decreasing the activity of EGR1 in the cell.

Embodiment 89. The method of embodiment 88, wherein decreasing the activity of EGR1 comprises decreasing the mRNA level of EGR1 in the cell.

Embodiment 90. The method of embodiment 89, wherein the mRNA level of EGR1 is decreased by at least 1%, 2%, 3%, 4%, 5%, or 10% as compared to a corresponding untreated cell. Embodiment 91. The method of embodiment 88, wherein decreasing the activity of EGR1 comprises decreasing the protein level of EGR1 in the cell.

Embodiment 92. The method of embodiment 89, wherein the protein level of EGR1 is decreased by at least 1%, 2%, 3%, 4%, 5%, or 10% as compared to a corresponding untreated cell. Embodiment 93. The method of embodiment 47, wherein the method comprises decreasing the activity of MAZ in the cell.

Embodiment 94. The method of embodiment 93, wherein decreasing the activity of MAZ comprises decreasing the mRNA level of MAZ in the cell.

Embodiment 95. The method of embodiment 94, wherein the mRNA level of MAZ is decreased by at least 1%, 2%, 3%, 4%, 5%, or 10% as compared to a corresponding untreated cell.

Embodiment 96. The method of embodiment 93, wherein decreasing the activity of MAZ comprises decreasing the protein level of MAZ in the cell.

Embodiment 97. The method of embodiment 96, wherein the protein level of MAZ is decreased by at least 1%, 2%, 3%, 4%, 5%, or 10% as compared to a corresponding untreated cell.

Embodiment 98. The method of embodiment 47, wherein the method comprises decreasing the activity of SOX2 in the cell.

Embodiment 99. The method of embodiment 98, wherein decreasing the activity of SOX2 comprises decreasing the mRNA level of SOX2 in the cell.

Embodiment 100. The method of embodiment 99, wherein the mRNA level of SOX2 is decreased by at least 1%, 2%, 3%, 4%, 5%, or 10% as compared to a corresponding untreated cell.

Embodiment 101. The method of embodiment 98, wherein decreasing the activity of SOX2 comprises decreasing the protein level of SOX2 in the cell.

Embodiment 102. The method of embodiment 101, wherein the protein level of SOX2 is decreased by at least 1%, 2%, 3%, 4%, 5%, or 10% as compared to a corresponding untreated cell.

Embodiment 103. The method of embodiment 47, wherein the method comprises decreasing the activity of ATF4 in the cell.

Embodiment 104. The method of embodiment 103, wherein decreasing the activity of ATF4 comprises decreasing the mRNA level of ATF4 in the cell.

Embodiment 105. The method of embodiment 104, wherein the mRNA level of ATF4 is decreased by at least 1%, 2%, 3%, 4%, 5%, or 10% as compared to a corresponding untreated cell.

Embodiment 106. The method of embodiment 103, wherein decreasing the activity of ATF4 comprises decreasing the protein level of ATF4 in the cell.

Embodiment 107. The method of embodiment 106, wherein the protein level of ATF4 is decreased by at least 1%, 2%, 3%, 4%, 5%, or 10% as compared to a corresponding untreated cell.

Embodiment 108. The method of any one of embodiments 1-107, wherein decreasing the activity of the at least one transcription factor comprises introducing into the cell one or more agents for decreasing transcription from the at least one transcription factor.

Embodiment 109. The method of embodiment 108, wherein the one or more agents comprise a CRISPR system, a repression plasmid, an antisense oligonucleotide, or siRNA.

Embodiment 110. The method of embodiment 108, wherein the one or more agents are one or more polynucleic acids.

Embodiment 111. The method of embodiment 110, wherein the one or more polynucleic acids encode the CRISPR system, the antisense oligonucleotide, or the siRNA.

Embodiment 112. The method of embodiment 110 or 111, wherein the one or more polynucleic acids are comprised in a viral vector.

Embodiment 113. The method of any one of embodiments 110 or 111, wherein the one or more polynucleic acids are comprised in a non-viral vector.

Embodiment 114. The method of any one of embodiments 110 or 111, wherein the one or more polynucleic acids are encapsulated in a lipid nanoparticle (LNP).

Embodiment 115. The method of any one of embodiments 1-114, wherein decreasing the activity of STAT3 comprises introducing into the cell an inhibitor of STAT3.

Embodiment 116. The method of any one of embodiments 1-115, wherein decreasing the activity of ZFX comprises introducing into the cell an inhibitor of ZFX.

Embodiment 117. The method of any one of embodiments 1-116, wherein the cell is ex vivo.

Embodiment 118. The method of embodiment 117, wherein the cell is from a human subject having an age of at least 30 years, at least 35 years, at least 40 years, at least 45 years, at least 50 years, at least 55 years, at least 60 years, at least 65 years, at least 70 years, at least 75 years, at least 80 years, at least 85 years, or at least 90 years.

Embodiment 119. The method of embodiment 118, wherein the human subject does not have psoriasis.

Embodiment 120. The method of any one of embodiments 1-119, wherein the cell is in situ.

Embodiment 121. The method of any one of embodiments 1-119, wherein the cell is in vivo.

Embodiment 122. The method of embodiment 120 or 121, wherein the cell is in a human subject having an age of at least 30 years, at least 35 years, at least 40 years, at least 45 years, at least 50 years, at least 55 years, at least 60 years, at least 65 years, at least 70 years, at least 75 years, at least 80 years, at least 85 years, or at least 90 years.

Embodiment 123. The method of embodiment 122, wherein the human subject does not have psoriasis and the cells are in situ.

Embodiment 124. The method of embodiment 122, wherein the human subject does not have psoriasis and the cells are in vivo.

Embodiment 125. The method of any one of embodiments 1-123, wherein the cell, after treatment, does not comprise substantial changes of TERT-related gene expression or telomere length. Exemplary TERT-related genes include EREG, EEF1A2, ALDH1A1, and EPB41L3.

Embodiment 126. The method of any one of embodiments 1-125, wherein the method does not comprise increasing the activity of any one of OCT4, SOX2, KLF4, and MYC.

Embodiment 127. The method of embodiment 126, wherein the method does not comprise increasing the activity of OCT4, SOX2, KLF4, and MYC.

Embodiment 128. A composition for rejuvenating cells, the composition comprising:
(i) at least one activator of a transcription factor, wherein the transcription factor is selected from the group consisting of:
   (a) E2F Transcription Factor 3 (E2F3);
   (b) Enhancer Of Zeste 2 Polycomb Repressive Complex 2 Subunit (EZH2);
   (c) Homeobox protein DLX-6 (DLX6);
   (d) Forkhead Box M1 (FOXM1);
   (e) FOS Like 1, AP-1 Transcription Factor Subunit (FOSL1);
   (f) Nuclear Factor Of Activated T Cells 4 (NFATC4);
   (g) Myc proto-oncogene protein (MYC);
   (h) Signal Transducer And Activator Of Transcription 4 (STAT4);
   (i) GATA Binding Protein 3 (GATA3);
   (j) Heat Shock Transcription Factor 2 (HSF2);
   (k) Paired Box 4 (PAX4);
   (l) NK2 Homeobox 2 (NKX2-2);
   (m) SIM BHLH Transcription Factor 2 (SIM2); and
   (n) Ventral Anterior Homeobox 1 (VAX1);
      and
(ii) at least one inhibitor of a transcription factor, wherein the transcription factor is selected from the group consisting of:
   (a) Signal Transducer And Activator Of Transcription 3 (STAT3);
   (b) Zinc Finger Protein X-Linked (ZFX);
   (c) Early growth response protein 1 (EGR1);
   (d) Activating Transcription Factor 2 (ATF2);
   (e) Vascular Endothelial Zinc Finger 1 (VEZF1);
   (f) MYC Associated Zinc Finger Protein (MAZ);
   (g) SRY-Box Transcription Factor 2 (SOX2);
   (h) Paired Box 8 (PAX8);
   (i) Zinc Finger Homeobox 3 (ZFHX3);
   (j) Autophagy Related 4C Cysteine Peptidase (ATG4C);
   (k) Autophagy Related 5 (ATG5);
   (l) High Mobility Group Box 1 (HMGB1);
   (m) GATA Binding Protein 2 (GATA2); and
   (n) KLF Transcription Factor 4 (KLF4).

Embodiment 129. The composition of embodiment 128, wherein the composition comprises:
(a) at least one activator of a transcription factor of EZH2, E2F3, FOXM1, or DLX6; and
(b) at least one inhibitor of a transcription factor of STAT3, ZFX, EGR1, MAZ, SOX2, or ATF4.

Embodiment 130. The composition of embodiment 128 or 129, wherein the composition does not comprise an activator of a transcription factor of OCT4, SOX2, KLF4, or MYC.

Embodiment 131. The composition of any one of embodiments 128-130, wherein the composition comprises (a) an activator of a transcription factor of EZH2 or E2F3; and (b) an inhibitor of a transcription factor of STAT3 or ZFX.

Embodiment 132. The composition of any one of embodiments 128-131, wherein the composition comprises an activator of E2F3 and an activator of EZH2.

Embodiment 133. The composition of any one of embodiments 128-131, wherein the composition comprises an inhibitor of STAT3 and an inhibitor of ZFX.

Embodiment 134. The composition of any one of embodiments 128-131, wherein the composition comprises an activator of E2F3 and an inhibitor of STAT3.

Embodiment 135. The composition of any one of embodiments 128-131, wherein the composition comprises an activator of E2F3 and an inhibitor of ZFX.

Embodiment 136. The composition of any one of embodiments 128-131, wherein the composition comprises an activator of EZH2 and an inhibitor of STAT3.

Embodiment 137. The composition of any one of embodiments 128-131, wherein the composition comprises an activator of EZH2 and an inhibitor of ZFX.

Embodiment 138. The composition of any one of embodiments 128-131, wherein the composition comprises an activator of E2F3, an activator of EZH2 and an inhibitor of STAT3.

Embodiment 139. The composition of any one of embodiments 128-131, wherein the composition comprises an activator of E2F3, an activator of EZH2 and an inhibitor of ZFX.

Embodiment 140. The composition of any one of embodiments 128-131, wherein the composition comprises an activator of EZH2, an inhibitor of STAT3, and an inhibitor of ZFX.

Embodiment 141. The composition of any one of embodiments 128-131, wherein the composition comprises an activator of E2F3, an activator of EZH2, an inhibitor of STAT3 and an inhibitor ZFX.

Embodiment 142. The composition of any one of embodiments 128-141, wherein the activator comprises a polynucleic acid encoding the transcription factor or an expression plasmid comprising the polynucleic acid encoding the transcription factor.

Embodiment 143. The composition of any one of embodiments 128-142, wherein the inhibitor comprises a CRISPR system, a repression plasmid, an antisense oligonucleotide, or siRNA targeting the transcription factor: or a polynucleic acid encoding the CRISPR system, the antisense oligonucleotide, or the siRNA.

Embodiment 144. A primary cell, comprising:
(i) at least one activator of a transcription factor, wherein the transcription factor is selected from the group consisting of:
   (a) E2F Transcription Factor 3 (E2F3);
   (b) Enhancer Of Zeste 2 Polycomb Repressive Complex 2 Subunit (EZH2);
   (c) Homeobox protein DLX-6 (DLX6);
   (d) Forkhead Box M1 (FOXM1);
   (e) FOS Like 1, AP-1 Transcription Factor Subunit (FOSL1);
   (f) Nuclear Factor Of Activated T Cells 4 (NFATC4);
   (g) Myc proto-oncogene protein (MYC);
   (h) Signal Transducer And Activator Of Transcription 4 (STAT4);
   (i) GATA Binding Protein 3 (GATA3);
   (j) Heat Shock Transcription Factor 2 (HSF2);
   (k) Paired Box 4 (PAX4);
   (l) NK2 Homeobox 2 (NKX2-2);
   (m) SIM BHLH Transcription Factor 2 (SIM2); and
   (n) Ventral Anterior Homeobox 1 (VAX1);
or
(ii) at least one inhibitor of a transcription factor, wherein the transcription factor is selected from the group consisting of:
   (a) Signal Transducer And Activator Of Transcription 3 (STAT3);
   (b) Zinc Finger Protein X-Linked (ZFX);
   (c) Early growth response protein 1 (EGR1);
   (d) Activating Transcription Factor 2 (ATF2);
   (e) Vascular Endothelial Zinc Finger 1 (VEZF1);
   (f) MYC Associated Zinc Finger Protein (MAZ);
   (g) SRY-Box Transcription Factor 2 (SOX2);
   (h) Paired Box 8 (PAX8);
   (i) Zinc Finger Homeobox 3 (ZFHX3);
   (j) Autophagy Related 4C Cysteine Peptidase (ATG4C);
   (k) Autophagy Related 5 (ATG5);
   (l) High Mobility Group Box 1 (HMGB1);
   (m) GATA Binding Protein 2 (GATA2); and
   (n) KLF Transcription Factor 4 (KLF4).

Embodiment 145. The primary cell of embodiment 144, wherein the primary cell is a primary human cell.

Embodiment 146. The primary cell of embodiment 144, wherein the primary cell is a primary adult human cell.

Embodiment 147. The primary cell of any one of embodiments 144-146, wherein the primary cell comprises:
(a) at least one activator of a transcription factor of EZH2, E2F3, FOXM1 or DLX6; or
(b) at least one inhibitor of a transcription factor of STAT3, ZFX, EGR1, MAZ, SOX2, or ATF4.

Embodiment 148. The primary cell of any one of embodiments 144-147, wherein the primary cell does not comprise an activator of a transcription factor of OCT4, SOX2, KLF4, or MYC.

Embodiment 149. The primary cell of any one of embodiments 144-148, wherein the primary cell comprises (a) an activator of a transcription factor of EZH2 or E2F3; or (b) an inhibitor of a transcription factor of STAT3 or ZFX.

Embodiment 150. The primary cell of any one of embodiments 144-149, wherein the primary cell comprises an activator of E2F3 and an activator of EZH2.

Embodiment 151. The primary cell of any one of embodiments 144-149, wherein the primary cell comprises an inhibitor of STAT3 and an inhibitor of ZFX.

Embodiment 152. The primary cell of any one of embodiments 144-149, wherein the primary cell comprises an activator of E2F3 and an inhibitor of STAT3.

Embodiment 153. The primary cell of any one of embodiments 144-149, wherein the primary cell comprises an activator of E2F3 and an inhibitor of ZFX.

Embodiment 154. The primary cell of any one of embodiments 144-149, wherein the primary cell comprises an activator of EZH2 and an inhibitor of STAT3.

Embodiment 155. The primary cell of any one of embodiments 144-149, wherein the primary cell comprises an activator of EZH2 and an inhibitor of ZFX.

Embodiment 156. The primary cell of any one of embodiments 144-149, wherein the primary cell comprises an activator of E2F3, an activator of EZH2 and an inhibitor of STAT3.

Embodiment 157. The primary cell of any one of embodiments 144-149, wherein the primary cell comprises an activator of E2F3, an activator of EZH2 and an inhibitor of ZFX.

Embodiment 158. The primary cell of any one of embodiments 144-149, wherein the primary cell comprises an activator of EZH2, an inhibitor of STAT3, and an inhibitor of ZFX.

Embodiment 159. The primary cell of any one of embodiments 144-149, wherein the primary cell comprises an activator of E2F3, an activator of EZH2, an inhibitor of STAT3 and an inhibitor ZFX.

Embodiment 160. The primary cell of any one of embodiments 144-159, wherein the activator comprises a polynucleic acid encoding the transcription factor or an expression plasmid comprising the polynucleic acid encoding the transcription factor.

Embodiment 161. The primary cell of any one of embodiments 144-160, wherein the inhibitor comprises a CRISPR system, a repression plasmid, an antisense oligonucleotide, or siRNA targeting the transcription factor; or a polynucleic acid encoding the CRISPR system, the antisense oligonucleotide, or the siRNA.

Embodiment 162. A pharmaceutical composition comprising
(i) a composition that comprises:
   (a) at least one activator of a transcription factor, wherein the transcription factor is selected from the group consisting of:
      (i) E2F Transcription Factor 3 (E2F3);
      (ii) Enhancer Of Zeste 2 Polycomb Repressive Complex 2 Subunit (EZH2);
      (iii) Homeobox protein DLX-6 (DLX6);
      (iv) Forkhead Box M1 (FOXM1);
      (v) FOS Like 1, AP-1 Transcription Factor Subunit (FOSL1);
      (vi) Nuclear Factor Of Activated T Cells 4 (NFATC4);
      (vii) Myc proto-oncogene protein (MYC);
      (viii) Signal Transducer And Activator Of Transcription 4 (STAT4);
      (ix) GATA Binding Protein 3 (GATA3);
      (x) Heat Shock Transcription Factor 2 (HSF2);
      (xi) Paired Box 4 (PAX4);
      (xii) NK2 Homeobox 2 (NKX2-2):
      (xiii) SIM BHLH Transcription Factor 2 (SIM2); and
      (xiv) Ventral Anterior Homeobox 1 (VAX1);
or
   (b) at least one inhibitor of a transcription factor, wherein the transcription factor is selected from the group consisting of:
      (i) Signal Transducer And Activator Of Transcription 3 (STAT3);
      (ii) Zinc Finger Protein X-Linked (ZFX):
      (iii) Early growth response protein 1 (EGR1);
      (iv) Activating Transcription Factor 2 (ATF2);
      (v) Vascular Endothelial Zinc Finger 1 (VEZF1);
      (vi) MYC Associated Zinc Finger Protein (MAZ);
      (vii) SRY-Box Transcription Factor 2 (SOX2);
      (viii) Paired Box 8 (PAX8);
      (ix) Zinc Finger Homeobox 3 (ZFHX3);
      (x) Autophagy Related 4C Cysteine Peptidase (ATG4C):
      (xi) Autophagy Related 5 (ATG5):
      (xii) High Mobility Group Box 1 (HMGB1);
      (xiii) GATA Binding Protein 2 (GATA2); and
      (xiv) KLF Transcription Factor 4 (KLF4); and
(ii) a pharmaceutically acceptable carrier, excipient, or diluent.

Embodiment 163. The pharmaceutical composition of embodiment 162, wherein the composition comprises:
(a) at least one activator of a transcription factor of EZH2, E2F3, FOXM1, or DLX6; or
(b) at least inhibitor of a transcription factor of STAT3, ZFX, EGR1, MAZ, SOX2, or ATF4.

Embodiment 164. The pharmaceutical composition of embodiment 162 or 163, wherein the composition does not comprise an activator of a transcription factor of OCT4, SOX2, KLF4, or MYC.

Embodiment 165. The pharmaceutical composition of any one of embodiments 162-164, wherein the composition comprises (a) an activator of a transcription factor of EZH2 or E2F3; or (b) an inhibitor of a transcription factor of STAT3 or ZFX.

Embodiment 166. The pharmaceutical composition of any one of embodiments 162-165, wherein the composition comprises an activator of E2F3 and an activator of EZH2.

Embodiment 167. The pharmaceutical composition of any one of embodiments 162-165, wherein the composition comprises an inhibitor of STAT3 and an inhibitor of ZFX.

Embodiment 168. The pharmaceutical composition of any one of embodiments 162-165, wherein the composition comprises an activator of E2F3 and an inhibitor of STAT3.

Embodiment 169. The pharmaceutical composition of any one of embodiments 162-165, wherein the composition comprises an activator of E2F3 and an inhibitor of ZFX.

Embodiment 170. The pharmaceutical composition of any one of embodiments 162-165, wherein the composition comprises an activator of EZH2 and an inhibitor of STAT3.

Embodiment 171. The pharmaceutical composition of any one of embodiments 162-165, wherein the composition comprises an activator of EZH2 and an inhibitor of ZFX.

Embodiment 172. The pharmaceutical composition of any one of embodiments 162-165, wherein the composition comprises an activator of E2F3, an activator of EZH2 and an inhibitor of STAT3.

Embodiment 173. The pharmaceutical composition of any one of embodiments 162-165, wherein the composition comprises an activator of E2F3, an activator of EZH2 and an inhibitor of ZFX.

Embodiment 174. The pharmaceutical composition of any one of embodiments 162-165, wherein the composition comprises an activator of EZH2, an inhibitor of STAT3, and an inhibitor of ZFX.

Embodiment 175. The pharmaceutical composition of any one of embodiments 162-165, wherein the composition comprises an activator of E2F3, an activator of EZH2, an inhibitor of STAT3 and an inhibitor ZFX.

Embodiment 176. The pharmaceutical composition of any one of embodiments 162-176, wherein the activator comprises a polynucleic acid encoding the transcription factor or an expression plasmid comprising the polynucleic acid encoding the transcription factor.

Embodiment 177. The pharmaceutical composition of any one of embodiments 162-176, wherein the inhibitor comprises a CRISPR system, a repression plasmid, an antisense oligonucleotide, or siRNA targeting the transcription factor; or a polynucleic acid encoding the CRISPR system, the antisense oligonucleotide, or the siRNA.

Embodiment 178. A LNP composition comprising The composition of any one of embodiments 128-143.

Embodiment 179. A cell comprising The composition of any one of embodiments 128-143.

Embodiment 180. A cell produced by the method of any one of embodiments 1-127.

Embodiment 181. A polynucleotide comprising The composition of any one of embodiments 128-178.

Embodiment 182. A pharmaceutical composition comprising (i) The composition of any one of embodiments 128-143, The primary cell of embodiment 144-161, the cell of embodiment 179 or embodiment 180, or the polynucleotide of embodiment 181, and (ii) a pharmaceutically acceptable carrier, excipient, or diluent.

Embodiment 183. A method of treating a disorder in a subject in need thereof, the method comprising administration of The pharmaceutical composition of any one of embodiments 162-177 or the pharmaceutical composition of embodiment 182 to the subject in need thereof.

Embodiment 184. The method of embodiment 183, wherein the disorder is an age-related disorder.

Embodiment 185. A kit, comprising:
(i) at least one activator of a transcription factor, wherein the transcription factor is selected from the group consisting of:
   (a) E2F Transcription Factor 3 (E2F3);
   (b) Enhancer Of Zeste 2 Polycomb Repressive Complex 2 Subunit (EZH2);
   (c) Homeobox protein DLX-6 (DLX6);
   (d) Forkhead Box M1 (FOXM1);
   (e) FOS Like 1, AP-1 Transcription Factor Subunit (FOSL1);
   (f) Nuclear Factor Of Activated T Cells 4 (NFATC4);
   (g) Myc proto-oncogene protein (MYC);
   (h) Signal Transducer And Activator Of Transcription 4 (STAT4);
   (i) GATA Binding Protein 3 (GATA3);
   (j) Heat Shock Transcription Factor 2 (HSF2);
   (k) Paired Box 4 (PAX4);
   (l) NK2 Homeobox 2 (NKX2-2);
   (m) SIM BHLH Transcription Factor 2 (SIM2); and
   (n) Ventral Anterior Homeobox 1 (VAX1);
      and
(ii) at least one inhibitor of a transcription factor, wherein the transcription factor is selected from the group consisting of:
   (a) Signal Transducer And Activator Of Transcription 3 (STAT3);
   (b) Zinc Finger Protein X-Linked (ZFX);
   (c) Early growth response protein 1 (EGR1);
   (d) Activating Transcription Factor 2 (ATF2);
   (e) Vascular Endothelial Zinc Finger 1 (VEZF1);
   (f) MYC Associated Zinc Finger Protein (MAZ);
   (g) SRY-Box Transcription Factor 2 (SOX2);
   (h) Paired Box 8 (PAX8);
   (i) Zinc Finger Homeobox 3 (ZFHX3);
   (j) Autophagy Related 4C Cysteine Peptidase (ATG4C);
   (k) Autophagy Related 5 (ATG5);
   (l) High Mobility Group Box 1 (HMGB1);
   (m) GATA Binding Protein 2 (GATA2); and
   (n) KLF Transcription Factor 4 (KLF4).

Embodiment 186. The kit of embodiment 185, wherein the kit comprises:
(a) at least one activator of a transcription factor of EZH2, E2F3, FOXM1, or DLX6; and
(b) at least one inhibitor of a transcription factor of STAT3, ZFX, EGR1, MAZ, SOX2, or ATF4.

Embodiment 187. The kit of embodiment 185 or 186, wherein the kit does not comprise an activator of a transcription factor of OCT4, SOX2, KLF4, or MYC.

Embodiment 188. The kit of any one of embodiments 185-187, wherein the kit comprises (a) one or both activator of a transcription factor of EZH2 or E2F3; and (b) one or both inhibitor of a transcription factor of STAT3 or ZFX.

Embodiment 189. The kit of any one of embodiments 185-188, wherein the kit comprises an activator of E2F3 and an activator of EZH2.

Embodiment 190. The kit of any one of embodiments 185-188, wherein the kit comprises an inhibitor of STAT3 and an inhibitor of ZFX.

Embodiment 191. The kit of any one of embodiments 185-188, wherein the kit comprises an activator of E2F3 and an inhibitor of STAT3.

Embodiment 192. The kit of any one of embodiments 185-188, wherein the kit comprises an activator of E2F3 and an inhibitor of ZFX.

Embodiment 193. The kit of any one of embodiments 185-188, wherein the kit comprises an activator of EZH2 and an inhibitor of STAT3.

Embodiment 194. The kit of any one of embodiments 185-188, wherein the kit comprises an activator of EZH2 and an inhibitor of ZFX.

Embodiment 195. The kit of any one of embodiments 185-188, wherein the kit comprises an activator of E2F3, an activator of EZH2 and an inhibitor of STAT3.

Embodiment 196. The kit of any one of embodiments 185-188, wherein the kit comprises an activator of E2F3, an activator of EZH2 and an inhibitor of ZFX.

Embodiment 197. The kit of any one of embodiments 185-188, wherein the kit comprises an activator of EZH2, an inhibitor of STAT3, and an inhibitor of ZFX.

Embodiment 198. The kit of any one of embodiments 185-188, wherein the kit comprises an activator of E2F3, an activator of EZH2, an inhibitor of STAT3 and an inhibitor ZFX.

Embodiment 199. The kit of any one of embodiments 185-198, wherein the activator comprises a polynucleic acid encoding the transcription factor or an expression plasmid comprising the polynucleic acid encoding the transcription factor.

Embodiment 200. The kit of any one of embodiments 185-199, wherein the inhibitor comprises a CRISPR system, a repression plasmid, an antisense oligonucleotide, or siRNA; or a polynucleic acid encoding the CRISPR system, the antisense oligonucleotide, or the siRNA.

Embodiment 201. A method for rejuvenating a cell, the method comprising increasing activity of one or more transcription factors in the cell, wherein the one or more transcription factors are selected from the group consisting of:
(a) E2F Transcription Factor 3 (E2F3);
(b) Enhancer Of Zeste 2 Polycomb Repressive Complex 2 Subunit (EZH2);
(c) Homeobox protein DLX-6 (DLX6);
(d) Forkhead Box M1 (FOXM1);
(e) FOS Like 1, AP-1 Transcription Factor Subunit (FOSL1);
(f) Nuclear Factor Of Activated T Cells 4 (NFATC4);
(g) Myc proto-oncogene protein (MYC);
(h) Signal Transducer And Activator Of Transcription 4 (STAT4);
(i) GATA Binding Protein 3 (GATA3);
(j) Heat Shock Transcription Factor 2 (HSF2);
(k) Paired Box 4 (PAX4);
(l) NK2 Homeobox 2 (NKX2-2);
(m) SIM BHLH Transcription Factor 2 (SIM2); and
(n) Ventral Anterior Homeobox 1 (VAX1);
   and/or
   wherein the method comprising decreasing activity of one or more transcription factors in the cell,
   wherein the transcription factors are selected from the group consisting of:
(i) Signal Transducer And Activator Of Transcription 3 (STAT3);
(ii) Zinc Finger Protein X-Linked (ZFX);
(iii) Early growth response protein 1 (EGR1);
(iv) Activating Transcription Factor 2 (ATF2);
(v) Vascular Endothelial Zinc Finger 1 (VEZF1);
(vi) MYC Associated Zinc Finger Protein (MAZ);
(vii) SRY-Box Transcription Factor 2 (SOX2);
(viii) Paired Box 8 (PAX8);
(ix) Zinc Finger Homeobox 3 (ZFHX3);
(x) Autophagy Related 4C Cysteine Peptidase (ATG4C);
(xi) Autophagy Related 5 (ATG5);
(xii) High Mobility Group Box 1 (HMGB1);
(xiii) GATA Binding Protein 2 (GATA2); and
(xiv) KLF Transcription Factor 4 (KLF4).

Embodiment 202. The method of embodiment 201, wherein the one or more transcription factors are selected from the group consisting of: STAT3, ZFX, EGR1, MAZ, SOX2, and ATF4.

Embodiment 203. The method of embodiment 201, wherein the method comprising increasing activity of one or more transcription factors in the cell, wherein the one or more transcription factors are selected from the group consisting of E2F3, EZH2, FOXM1, and DLX6; and wherein the method comprising decreasing activity of one or more transcription factors in the cell, wherein the transcription factors are selected from the group consisting of STAT3, ZFX, EGR1, MAZ, SOX2, and ATF4.

Embodiment 204. The method of embodiment 203, wherein the method comprising increasing activity of one or more transcription factors in the cell, wherein the one or more transcription factors are E2F3 or EZH2; and wherein the method comprising decreasing activity of one or more transcription factors in the cell, wherein the transcription factors are STAT3 or ZFX.

Embodiment 205. The method of any one of embodiments 201-204, wherein the method comprises increasing the activities of E2F3 and EZH2.

Embodiment 206. The method of any one of embodiments 201-204, wherein the method comprises decreasing the activities of STAT3 and ZFX.

Embodiment 207. The method of any one of embodiments 201-204, wherein the method comprises increasing the activity of E2F3 and decreasing the activity of STAT3.

Embodiment 208. The method of any one of embodiments 201-204, wherein the method comprises increasing the activity of E2F3 and decreasing the activity of ZFX.

Embodiment 209. The method of any one of embodiments 201-204, wherein the method comprises increasing the activity of EZH2 and decreasing the activity of STAT3.

Embodiment 210. The method of any one of embodiments 201-204, wherein the method comprises increasing the activities of EZH2 and decreasing the activity of ZFX.

Embodiment 211. The method of any one of embodiments 201-204, wherein the method comprises increasing the activities of E2F3 and EZH2 and decreasing the activity of STAT3.

Embodiment 212. The method of any one of embodiments 201-204, wherein the method comprises increasing the activities of E2F3 and EZH2 and decreasing the activity of ZFX.

Embodiment 213. The method of any one of embodiments 201-204, wherein the method comprises increasing the activity of EZH2 and decreasing the activities of STAT3 and ZFX.

Embodiment 214. The method of any one of embodiments 201-204, wherein the method comprises increasing the activities of E2F3 and EZH2 and decreasing the activities of STAT3 and ZFX.

Embodiment 215. The method of embodiment 201, wherein the activity of any two of the transcription factors is increased.

Embodiment 216. The method of embodiment 201, wherein the activity of at least two of the transcription factors is increased.

Embodiment 217. The method of embodiment 201, wherein the activity of any two of the transcription factors is decreased.

Embodiment 218. The method of embodiment 201, wherein the activity of at least two of the transcription factors is decreased.

Embodiment 219. The method of embodiment 201, wherein the activity of any two of the transcription factors is increased or decreased.

Embodiment 220. The method of embodiment 201, wherein the activity of at least two of the transcription factors is increased.

What is claimed is:

1. A method for rejuvenating a cell, the method comprising:
(i) increasing the activity of at least one transcription factor selected from:
(i) E2F Transcription Factor 3 (E2F3); and
(ii) Enhancer Of Zeste 2 Polycomb Repressive Complex 2 Subunit (EZH2); or
(ii) decreasing the activity of at least one transcription factor selected from:
(i) Signal Transducer And Activator Of Transcription 3 (STAT3); and
(ii) Zinc Finger Protein X-Linked (ZFX).

2. The method of claim 1, wherein the method comprises:
(i) increasing the activity of at least one transcription factor selected from:
(i) E2F Transcription Factor 3 (E2F3); and
(ii) Enhancer Of Zeste 2 Polycomb Repressive Complex 2 Subunit (EZH2); and
(ii) decreasing the activity of at least one transcription factor selected from:
(i) Signal Transducer And Activator Of Transcription 3 (STAT3); and
(ii) Zinc Finger Protein X-Linked (ZFX).

3. The method of claim 1, wherein the level of the activity of the at least one transcription factor is increased or decreased, thereby rejuvenating the cell.

4. The method of claim 1, wherein the method comprises increasing the activities of E2F3 and EZH2.

5. The method of claim 1, wherein the method comprises decreasing the activities of STAT3 and ZFX.

6. The method of claim 1, wherein the method comprises increasing the activity of E2F3 and decreasing the activity of STAT3.

7. The method of claim 1, wherein the method comprises increasing the activity of E2F3 and decreasing the activity of ZFX.

8. The method of claim 1, wherein the method comprises increasing the activity of EZH2 and decreasing the activity of STAT3.

9. The method of claim 1, wherein the method comprises increasing the activities of EZH2 and decreasing the activity of ZFX.

10. The method of claim 1, wherein the method comprises increasing the activities of E2F3 and EZH2 and decreasing the activity of STAT3.

11. The method of claim 1, wherein the method comprises increasing the activities of E2F3 and EZH2 and decreasing the activity of ZFX.

12. The method of claim 1, wherein the method comprises increasing the activity of EZH2 and decreasing the activities of STAT3 and ZFX.

13. The method of claim 1, wherein the method comprises increasing the activities of E2F3 and EZH2 and decreasing the activities of STAT3 and ZFX.

14. The method of claim 1, wherein the cell is a human cell.

15. The method of claim 1, wherein the cell is an adult human cell.

16. The method of claim 1, wherein the cell is a primary human cell.

17. The method of claim 1, wherein the cell is a primary adult human cell.

18. The method of claim 1, wherein the cell is a differentiated cell.

19. The method of claim 1, wherein the cell is an epithelial cell.

20. The method of claim 1, wherein the cell is a skin cell, a lung cell, a liver cell, a muscle cell, a pancreatic cell, an immune cell, a bone cell, or a brain cell.

21. The method of claim 1, wherein the cell is a fibroblast.

22. The method of claim 21, wherein the cell is a skin fibroblast.

23. The method of claim 21, wherein the cell is a liver fibroblast.

24. The method of claim 1, wherein the method results in an increase in cell division from the cell as compared to a corresponding untreated cell.

25. The method of claim 24, wherein the method results in at least 1%, 2%, 3%, 4%, or 5% increase in cell division.

26. The method of claim 1, wherein the method results in a cell that is capable of increasing cell division as compared to a corresponding untreated cell.

27. The method of claim 26, wherein the cell is capable of increasing cell division by at least 1%, 2%, 3%, 4%, or 5%.

28. The method of claim 24, wherein the increase in cell division is determined by an increase of the number of K167 positive cells in a cell population.

29. The method of claim 1, wherein the method results in a decrease in expression of at least one senescence related gene in the cell as compared to a corresponding untreated cell.

30. The method of claim 29, wherein the at least one senescence related gene is selected from the group consisting of p21, TIMP1, and TIMP2.

31. The method of claim 29, wherein the method results in at least 1%, 2%, 3%, 4%, or 5% decrease in expression of the at least one senescence related gene.

32. The method of claim 29, wherein the decrease in expression of the at least one senescence related gene is determined by an increase of the number of beta-galactosidase-positive cells.

33. The method of claim 1, wherein the method results in an increase in proteasome activity in the cell as compared to a corresponding untreated cell.

34. The method of claim 33, wherein the method results in at least 1%, 2%, 3%, 4%, or 5% increase in the proteasome activity.

35. The method of claim 1, wherein the method results in a cell that is capable of increasing proteasome activity as compared to a corresponding untreated cell.

36. The method of claim 35, wherein the cell is capable of increasing proteasome activity by at least 1%, 2%, 3%, 4%, or 5%.

37. The method of claim 33, wherein the increased proteasome activity is determined by an increase of proteasome-mediated cleavage of a substrate.

38. The method of claim 37, wherein the increased proteasome activity is measured by a fluorescence-based cleavage assay.

39. The method of claim 1, wherein the method results in a decrease in senescence-associated lysosomes in the cell as compared to a corresponding untreated cell.

40. The method of claim 39, wherein the method results in at least 1%, 2%, 3%, 4%, or 5% decrease in the senescence-associated lysosomes.

41. The method of claim 39, wherein the decrease in the senescence-associated lysosomes is determined by a decrease of the number of lysosome puncta per cell area.

42. The method of claim 41, wherein the number of lysosome puncta per cell area is measured by Lysotracker™ staining.

43. The method of claim 1, wherein the method results in an increase in mitochondrial and metabolism gene expression in the cell as compared to a corresponding untreated human adult cell.

44. The method of claim 1, wherein the method results in an increase in mitochondrial membrane potential in the cell as compared to a corresponding untreated cell.

45. The method of claim 44, wherein the increase in mitochondrial membrane potential is measured by TMRE (tetramethylrhodamine, ethyl ester) membrane potential staining.

46. The method of claim 1, wherein the method further comprises increasing the activity of at least one transcription factor selected from:
(i) Homeobox protein DLX-6 (DLX6);
(ii) Forkhead Box M1 (FOXM1);
(iii) FOS Like 1, AP-1 Transcription Factor Subunit (FOSL1);
(iv) Nuclear Factor Of Activated T Cells 4 (NFATC4);
(v) Myc proto-oncogene protein (MYC);
(vi) Signal Transducer And Activator Of Transcription 4 (STAT4);
(vii) GATA Binding Protein 3 (GATA3);
(viii) Heat Shock Transcription Factor 2 (HSF2);
(ix) Paired Box 4 (PAX4);
(x) NK2 Homeobox 2 (NKX2-2); and
(xi) SIM BHLH Transcription Factor 2 (SIM2).

47. The method of claim 1, wherein the method further comprises decreasing the activity of at least one transcription factor selected from:
(i) Early growth response protein 1 (EGR1);
(ii) Activating Transcription Factor 2 (ATF2);
(iii) Vascular Endothelial Zinc Finger 1 (VEZF1);
(iv) MYC Associated Zinc Finger Protein (MAZ);
(v) SRY-Box Transcription Factor 2 (SOX2);
(vi) Paired Box 8 (PAX8);
(vii) Zinc Finger Homeobox 3 (ZFHX3);
(viii) Autophagy Related 4C Cysteine Peptidase (ATG4C);
(ix) Autophagy Related 5 (ATG5);
(x) High Mobility Group Box 1 (HMGB1);
(xi) GATA Binding Protein 2 (GATA2); and
(xii) KLF Transcription Factor 4 (KLF4).

48. The method of claim 1, wherein the method comprises increasing the activity of E2F3 in the cell.

49. The method of claim 48, wherein increasing the activity of E2F3 comprises increasing the mRNA level of E2F3 in the cell.

50. The method of claim 49, wherein the mRNA level of E2F3 is increased by at least 1%, 2%, 3%, 4%, 5%, or 10% as compared to a corresponding untreated cell.

51. The method of claim 48, wherein increasing the activity of E2F3 comprising increasing the protein level of E2F3 in the cell.

52. The method of claim 51, wherein the protein E2F3 is increased by at least 1%, 2%, 3%, 4%, 5%, or 10% as compared to a corresponding untreated cell.

53. The method of claim 1, wherein the method comprises increasing the activity of EZH2 in the cell.

54. The method of claim 53, wherein increasing the activity of EZH2 comprises increasing the mRNA level of EZH2 in the cell.

55. The method of claim 54, wherein the mRNA level of EZH2 is increased by at least 1%, 2%, 3%, 4%, 5%, or 10% as compared to a corresponding untreated cell.

56. The method of claim 53, wherein increasing the activity of EZH2 comprises increasing the protein level of EZH2 in the cell.

57. The method of claim 56, wherein the protein level of EZH2 is increased by at least 1%, 2%, 3%, 4%, 5%, or 10% as compared to a corresponding untreated cell.

58. The method of claim 46, wherein the method comprises increasing the activity of DLX6 in the cell.

59. The method of claim 58, wherein increasing the activity of DLX6 comprises increasing the mRNA level of DLX6 in the cell.

60. The method of claim 59, wherein the mRNA level of DLX6 is increased by at least 1%, 2%, 3%, 4%, 5%, or 10% as compared to a corresponding untreated cell.

61. The method of claim 58, wherein increasing the activity of DLX6 comprises increasing the protein level of DLX6 in the cell.

62. The method of claim 61, wherein the mRNA level of DLX6 is increased by at least 1%, 2%, 3%, 4%, 5%, or 10% as compared to a corresponding untreated cell.

63. The method of claim 46, wherein the method comprises increasing the activity of FOXM1 in the cell.

64. The method of claim 63, wherein increasing the activity of FOXM1 comprises increasing the mRNA level of FOXM1 in the cell.

65. The method of claim 64, wherein the mRNA level of FOXM1 is increased by at least 1%, 2%, 3%, 4%, 5%, or 10% as compared to a corresponding untreated cell.

66. The method of claim 63, wherein increasing the activity of FOXM1 comprising increasing the protein level of FOXM1 in the cell.

67. The method of claim 66, wherein the protein FOXM1 is increased by at least 1%, 2%, 3%, 4%, 5%, or 10% as compared to a corresponding untreated cell.

68. The method of claim 1, wherein increasing the activity of the at least one transcription factor comprises introducing into the cell one or more polynucleic acids encoding the at least one transcription factor, thereby increasing transcription from the at least one transcription factor.

69. The method of claim 48, wherein increasing the activity of E2F3 comprises introducing into the cell one or more polynucleic acids encoding E2F3.

70. The method of claim 53, wherein increasing the activity of EZH2 comprises introducing into the cell one or more polynucleic acids encoding EZH2.

71. The method of claim 58, wherein increasing the activity of DLX6 comprises introducing into the cell one or more polynucleic acids encoding DLX6.

72. The method of claim 63, wherein increasing the activity of FOXM1 comprises introducing into the cell one or more polynucleic acids encoding FOXM1.

73. The method of claim 68, wherein the method comprising introducing into the cell (i) one or more polynucleic acids encoding E2F3, and (ii) one or more polynucleic acids encoding EZH2.

74. The method of claim 73, wherein the method further comprises introducing into the cell one or more polynucleic acids encoding DLX6 or one or more polynucleic acids encoding FOXM1.

75. The method of claim 68, wherein the one or more polynucleic acids are comprised in a viral vector.

76. The method of claim 68, wherein the one or more polynucleic acids are comprised in a non-viral vector.

77. The method of claim 68, wherein the one or more polynucleic acids are encapsulated in a lipid nanoparticle (LNP).

78. The method of claim 1, wherein the method comprises decreasing the activity of STAT3 in the cell.

79. The method of claim 78, wherein decreasing the activity of STAT3 comprises decreasing the mRNA level of STAT3 in the cell.

80. The method of claim 79, wherein the mRNA level of STAT3 is decreased by at least 1%, 2%, 3%, 4%, 5%, or 10% as compared to a corresponding untreated cell.

81. The method of claim 78, wherein decreasing the activity of STAT3 comprises decreasing the protein level of STAT3 in the cell.

82. The method of claim 81, wherein the protein level of STAT3 is decreased by at least 1%, 2%, 3%, 4%, 5%, or 10% as compared to a corresponding untreated cell.

83. The method of claim 1 wherein the method comprises decreasing the activity of ZFX in the cell.

84. The method of claim 83, wherein decreasing the activity of ZFX comprises decreasing the mRNA level of ZFX in the cell.

85. The method of claim 84, wherein the mRNA level of ZFX is decreased by at least 1%, 2%, 3%, 4%, 5%, or 10% as compared to a corresponding untreated cell.

86. The method of claim 83, wherein decreasing the activity of ZFX comprises decreasing the protein level of ZFX in the cell.

87. The method of claim 86, wherein the protein level of ZFX is decreased by at least 1%, 2%, 3%, 4%, 5%, or 10% as compared to a corresponding untreated cell.

88. The method of claim 46, wherein the method comprises decreasing the activity of EGR1 in the cell.

89. The method of claim 88, wherein decreasing the activity of EGR1 comprises decreasing the mRNA level of EGR1 in the cell.

90. The method of claim 89, wherein the mRNA level of EGR1 is decreased by at least 1%, 2%, 3%, 4%, 5%, or 10% as compared to a corresponding untreated cell.

91. The method of claim 88, wherein decreasing the activity of EGR1 comprises decreasing the protein level of EGR1 in the cell.

92. The method of claim 89, wherein the protein level of EGR1 is decreased by at least 1%, 2%, 3%, 4%, 5%, or 10% as compared to a corresponding untreated cell.

93. The method of claim 46, wherein the method comprises decreasing the activity of MAZ in the cell.

94. The method of claim 93, wherein decreasing the activity of MAZ comprises decreasing the mRNA level of MAZ in the cell.

95. The method of claim 94, wherein the mRNA level of MAZ is decreased by at least 1%, 2%, 3%, 4%, 5%, or 10% as compared to a corresponding untreated cell.

96. The method of claim 93, wherein decreasing the activity of MAZ comprises decreasing the protein level of MAZ in the cell.

97. The method of claim 96, wherein the protein level of MAZ is decreased by at least 1%, 2%, 3%, 4%, 5%, or 10% as compared to a corresponding untreated cell.

98. The method of claim 46, wherein the method comprises decreasing the activity of SOX2 in the cell.

99. The method of claim 98, wherein decreasing the activity of SOX2 comprises decreasing the mRNA level of SOX2 in the cell.

100. The method of claim 99, wherein the mRNA level of SOX2 is decreased by at least 1%, 2%, 3%, 4%, 5%, or 10% as compared to a corresponding untreated cell.

101. The method of claim 98, wherein decreasing the activity of SOX2 comprises decreasing the protein level of SOX2 in the cell.

102. The method of claim 101, wherein the protein level of SOX2 is decreased by at least 1%, 2%, 3%, 4%, 5%, or 10% as compared to a corresponding untreated cell.

103. The method of claim 46, wherein the method comprises decreasing the activity of ATF4 in the cell.

104. The method of claim 103, wherein decreasing the activity of ATF4 comprises decreasing the mRNA level of ATF4 in the cell.

105. The method of claim 104, wherein the mRNA level of ATF4 is decreased by at least 1%, 2%, 3%, 4%, 5%, or 10% as compared to a corresponding untreated cell.

106. The method of claim 103, wherein decreasing the activity of ATF4 comprises decreasing the protein level of ATF4 in the cell.

107. The method of claim 106, wherein the protein level of ATF4 is decreased by at least 1%, 2%, 3%, 4%, 5%, or 10% as compared to a corresponding untreated cell.

108. The method of claim 1, wherein decreasing the activity of the at least one transcription factor comprises introducing into the cell one or more agents for decreasing transcription from the at least one transcription factor.

109. The method of claim 108, wherein the one or more agents comprise a CRISPR system, a repression plasmid, an antisense oligonucleotide, or siRNA.

110. The method of claim 108, wherein the one or more agents are one or more polynucleic acids.

111. The method of claim 110, wherein the one or more polynucleic acids encode the CRISPR system, the antisense oligonucleotide, or the siRNA.

112. The method of claim 110, wherein the one or more polynucleic acids are comprised in a viral vector.

113. The method of claim 110, wherein the one or more polynucleic acids are comprised in a non-viral vector.

114. The method of claim 110 or 111, wherein the one or more polynucleic acids are encapsulated in a lipid nanoparticle (LNP).

115. The method of claim 1, wherein decreasing the activity of STAT3 comprises introducing into the cell an inhibitor of STAT3.

116. The method of claim 1, wherein decreasing the activity of ZFX comprises introducing into the cell an inhibitor of ZFX.

117. The method of claim 1, wherein the cell is ex vivo.

118. The method of claim 117, wherein the cell is from a human subject having an age of at least 30 years, at least 35 years, at least 40 years, at least 45 years, at least 50 years, at least 55 years, at least 60 years, at least 65 years, at least 70 years, at least 75 years, at least 80 years, at least 85 years, or at least 90 years.

119. The method of claim 118, wherein the human subject does not have psoriasis.

120. The method of claim 1, wherein the cell is in situ.

121. The method of claim 1, wherein the cell is in vivo.

122. The method of claim 120, wherein the cell is in a human subject having an age of at least 30 years, at least 35 years, at least 40 years, at least 45 years, at least 50 years, at least 55 years, at least 60 years, at least 65 years, at least 70 years, at least 75 years, at least 80 years, at least 85 years, or at least 90 years.

123. The method of claim 122, wherein the human subject does not have psoriasis and the cells are in situ.

124. The method of claim 122, wherein the human subject does not have psoriasis and the cells are in vivo.

125. The method of claim 1, wherein the cell, after treatment, does not comprise substantial changes of TERT-related gene expression or telomere length.

126. The method of claim 1, wherein the method does not comprise increasing the activity of any one of OCT4, SOX2, KLF4, and MYC.

127. The method of claim 126, wherein the method does not comprise increasing the activity of OCT4, SOX2, KLF4, and MYC.

128. A composition for rejuvenating cells, the composition comprising:
(i) at least one activator of a transcription factor, wherein the transcription factor is selected from the group consisting of:
(i) E2F Transcription Factor 3 (E2F3);
(ii) Enhancer Of Zeste 2 Polycomb Repressive Complex 2 Subunit (EZH2):
(iii) Homeobox protein DLX-6 (DLX6);
(iv) Forkhead Box M1 (FOXM1);
(v) FOS Like 1, AP-1 Transcription Factor Subunit (FOSL1);
(vi) Nuclear Factor Of Activated T Cells 4 (NFATC4);
(vii) Myc proto-oncogene protein (MYC);
(viii) Signal Transducer And Activator Of Transcription 4 (STAT4);
(ix) GATA Binding Protein 3 (GATA3);
(x) Heat Shock Transcription Factor 2 (HSF2);
(xi) Paired Box 4 (PAX4);
(xii) NK2 Homeobox 2 (NKX2-2);
(xiii) SIM BHLH Transcription Factor 2 (SIM2); and
(xiv) Ventral Anterior Homeobox 1 (VAX1);
and
(ii) at least one inhibitor of a transcription factor, wherein the transcription factor is selected from the group consisting of:
(i) Signal Transducer And Activator Of Transcription 3 (STAT3);
(ii) Zinc Finger Protein X-Linked (ZFX);
(iii) Early growth response protein 1 (EGR1);
(iv) Activating Transcription Factor 2 (ATF2);
(v) Vascular Endothelial Zinc Finger 1 (VEZF1);
(vi) MYC Associated Zinc Finger Protein (MAZ);
(vii) SRY-Box Transcription Factor 2 (SOX2);
(viii) Paired Box 8 (PAX8);
(ix) Zinc Finger Homeobox 3 (ZFHX3);
(x) Autophagy Related 4C Cysteine Peptidase (ATG4C);
(xi) Autophagy Related 5 (ATG5);
(xii) High Mobility Group Box 1 (HMGB1);
(xiii) GATA Binding Protein 2 (GATA2); and
(xiv) KLF Transcription Factor 4 (KLF4).

129. The composition of claim 128, wherein the composition comprises:
(a) at least one activator of a transcription factor of EZH2, E2F3, FOXM1, or DLX6; and
(b) at least one inhibitor of a transcription factor of STAT3, ZFX, EGR1, MAZ, SOX2, or ATF4.

130. The composition of claim 128, wherein the composition does not comprise an activator of a transcription factor of OCT4, SOX2, KLF4, or MYC.

131. The composition of claim 128, wherein the composition comprises (a) an activator of a transcription factor of EZH2 or E2F3; and (b) an inhibitor of a transcription factor of STAT3 or ZFX.

132. The composition of claim 128, wherein the composition comprises an activator of E2F3 and an activator of EZH2.

133. The composition of claim 128, wherein the composition comprises an inhibitor of STAT3 and an inhibitor of ZFX.

134. The composition of claim 128, wherein the composition comprises an activator of E2F3 and an inhibitor of STAT3.

135. The composition of claim 128, wherein the composition comprises an activator of E2F3 and an inhibitor of ZFX.

136. The composition of claim 128, wherein the composition comprises an activator of EZH2 and an inhibitor of STAT3.

137. The composition of claim 128, wherein the composition comprises an activator of EZH2 and an inhibitor of ZFX.

138. The composition of claim 128, wherein the composition comprises an activator of E2F3, an activator of EZH2 and an inhibitor of STAT3.

139. The composition of claim 128, wherein the composition comprises an activator of E2F3, an activator of EZH2 and an inhibitor of ZFX.

140. The composition of claim 128, wherein the composition comprises an activator of EZH2, an inhibitor of STAT3, and an inhibitor of ZFX.

141. The composition of claim 128, wherein the composition comprises an activator of E2F3, an activator of EZH2, an inhibitor of STAT3 and an inhibitor ZFX.

142. The composition of claim 128, wherein the activator comprises a polynucleic acid encoding the transcription factor or an expression plasmid comprising the polynucleic acid encoding the transcription factor.

143. The composition of claim 128, wherein the inhibitor comprises a CRISPR system, a repression plasmid, an antisense oligonucleotide, or siRNA targeting the transcription factor; or a polynucleic acid encoding the CRISPR system, the antisense oligonucleotide, or the siRNA.

144. A primary cell, comprising:
(i) at least one activator of a transcription factor, wherein the transcription factor is selected from the group consisting of:
(i) E2F Transcription Factor 3 (E2F3);
(ii) Enhancer Of Zeste 2 Polycomb Repressive Complex 2 Subunit (EZH2):
(iii) Homeobox protein DLX-6 (DLX6);
(iv) Forkhead Box M1 (FOXM1);
(v) FOS Like 1, AP-1 Transcription Factor Subunit (FOSL1);
(vi) Nuclear Factor Of Activated T Cells 4 (NFATC4);
(vii) Myc proto-oncogene protein (MYC);
(viii) Signal Transducer And Activator Of Transcription 4 (STAT4);
(ix) GATA Binding Protein 3 (GATA3);
(x) Heat Shock Transcription Factor 2 (HSF2);
(xi) Paired Box 4 (PAX4);
(xii) NK2 Homeobox 2 (NKX2-2);
(xiii) SIM BHLH Transcription Factor 2 (SIM2); and
(xiv) Ventral Anterior Homeobox 1 (VAX1);
or
(ii) at least one inhibitor of a transcription factor, wherein the transcription factor is selected from the group consisting of:
(i) Signal Transducer And Activator Of Transcription 3 (STAT3);
(ii) Zinc Finger Protein X-Linked (ZFX);
(iii) Early growth response protein 1 (EGR1);
(iv) Activating Transcription Factor 2 (ATF2);
(v) Vascular Endothelial Zinc Finger 1 (VEZF1);
(vi) MYC Associated Zinc Finger Protein (MAZ);
(vii) SRY-Box Transcription Factor 2 (SOX2);
(viii) Paired Box 8 (PAX8);
(ix) Zinc Finger Homeobox 3 (ZFHX3);
(x) Autophagy Related 4C Cysteine Peptidase (ATG4C);
(xi) Autophagy Related 5 (ATG5);
(xii) High Mobility Group Box 1 (HMGB1);
(xiii) GATA Binding Protein 2 (GATA2); and
(xiv) KLF Transcription Factor 4 (KLF4).

145. The primary cell of claim 144, wherein the primary cell is a primary human cell.

146. The primary cell of claim 144, wherein the primary cell is a primary adult human cell.

147. The primary cell of claim 144, wherein the primary cell comprises:
(a) at least one activator of a transcription factor of EZH2, E2F3, FOXM1 or DLX6;
or
(b) at least one inhibitor of a transcription factor of STAT3, ZFX, EGR1, MAZ, SOX2, or ATF4.

148. The primary cell of claim 144, wherein the primary cell does not comprise an activator of a transcription factor of OCT4, SOX2, KLF4, or MYC.

149. The primary cell of claim 144, wherein the primary cell comprises (a) an activator of a transcription factor of EZH2 or E2F3; or (b) an inhibitor of a transcription factor of STAT3 or ZFX.

150. The primary cell of claim 144, wherein the primary cell comprises an activator of E2F3 and an activator of EZH2.

151. The primary cell of claim 144, wherein the primary cell comprises an inhibitor of STAT3 and an inhibitor of ZFX.

152. The primary cell of claim 144, wherein the primary cell comprises an activator of E2F3 and an inhibitor of STAT3.

153. The primary cell of claim 144, wherein the primary cell comprises an activator of E2F3 and an inhibitor of ZFX.

154. The primary cell of claim 144, wherein the primary cell comprises an activator of EZH2 and an inhibitor of STAT3.

155. The primary cell of claim 144, wherein the primary cell comprises an activator of EZH2 and an inhibitor of ZFX.

156. The primary cell of claim 144, wherein the primary cell comprises an activator of E2F3, an activator of EZH2 and an inhibitor of STAT3.

157. The primary cell of claim 144, wherein the primary cell comprises an activator of E2F3, an activator of EZH2 and an inhibitor of ZFX.

158. The primary cell of claim 144, wherein the primary cell comprises an activator of EZH2, an inhibitor of STAT3, and an inhibitor of ZFX.

159. The primary cell of claim 144, wherein the primary cell comprises an activator of E2F3, an activator of EZH2, an inhibitor of STAT3 and an inhibitor ZFX.

160. The primary cell of claim 144, wherein the activator comprises a polynucleic acid encoding the transcription factor or an expression plasmid comprising the polynucleic acid encoding the transcription factor.

161. The primary cell of claim 144, wherein the inhibitor comprises a CRISPR system, a repression plasmid, an antisense oligonucleotide, or siRNA targeting the transcription factor; or a polynucleic acid encoding the CRISPR system, the antisense oligonucleotide, or the siRNA.

162. A pharmaceutical composition comprising (a) a composition that comprises:
(i) at least one activator of a transcription factor, wherein the transcription factor is selected from the group consisting of:
(a) E2F Transcription Factor 3 (E2F3);
(b) Enhancer Of Zeste 2 Polycomb Repressive Complex 2 Subunit (EZH2);
(c) Homeobox protein DLX-6 (DLX6);
(d) Forkhead Box M1 (FOXM1);
(e) FOS Like 1, AP-1 Transcription Factor Subunit (FOSL1);
(f) Nuclear Factor Of Activated T Cells 4 (NFATC4);
(g) Myc proto-oncogene protein (MYC);
(h) Signal Transducer And Activator Of Transcription 4 (STAT4);
(i) GATA Binding Protein 3 (GATA3);
(j) Heat Shock Transcription Factor 2 (HSF2);
(k) Paired Box 4 (PAX4);
(1) NK2 Homeobox 2 (NKX2-2);
(m) SIM BHLH Transcription Factor 2 (SIM2); and
(n) Ventral Anterior Homeobox 1 (VAX1);
or
(ii) at least one inhibitor of a transcription factor, wherein the transcription factor is selected from the group consisting of:
(a) Signal Transducer And Activator Of Transcription 3 (STAT3);
(b) Zinc Finger Protein X-Linked (ZFX);
(c) Early growth response protein 1 (EGR1);
(d) Activating Transcription Factor 2 (ATF2);
(e) Vascular Endothelial Zinc Finger 1 (VEZF1);
(f) MYC Associated Zinc Finger Protein (MAZ);
(g) SRY-Box Transcription Factor 2 (SOX2);
(h) Paired Box 8 (PAX8);
(i) Zinc Finger Homeobox 3 (ZFHX3);
j) Autophagy Related 4C Cysteine Peptidase (ATG4C);
(k) Autophagy Related 5 (ATG5);
(l) High Mobility Group Box 1 (HMGB1);
(m) GATA Binding Protein 2 (GATA2); and
(n) KLF Transcription Factor 4 (KLF4); and
(b) a pharmaceutically acceptable carrier, excipient, or diluent.

163. The pharmaceutical composition of claim 162, wherein the composition comprises:
(a) at least one activator of a transcription factor of EZH2, E2F3, FOXM1, or DLX6: or
(b) at least one inhibitor of a transcription factor of STAT3, ZFX, EGR1, MAZ, SOX2, or ATF4.

164. The pharmaceutical composition of claim 162, wherein the composition does not comprise an activator of a transcription factor of OCT4, SOX2, KLF4, or MYC.

165. The pharmaceutical composition of claim 162, wherein the composition comprises (a) an activator of a transcription factor of EZH2 or E2F3; or (b) an inhibitor of a transcription factor of STAT3 or ZFX.

166. The pharmaceutical composition of claim 162, wherein the composition comprises an activator of E2F3 and an activator of EZH2.

167. The pharmaceutical composition of claim 162, wherein the composition comprises an inhibitor of STAT3 and an inhibitor of ZFX.

168. The pharmaceutical composition of claim 162, wherein the composition comprises an activator of E2F3 and an inhibitor of STAT3.

169. The pharmaceutical composition of claim 162, wherein the composition comprises an activator of E2F3 and an inhibitor of ZFX.

170. The pharmaceutical composition of claim 162, wherein the composition comprises an activator of EZH2 and an inhibitor of STAT3.

171. The pharmaceutical composition of claim 162, wherein the composition comprises an activator of EZH2 and an inhibitor of ZFX.

172. The pharmaceutical composition of claim 162, wherein the composition comprises an activator of E2F3, an activator of EZH2 and an inhibitor of STAT3.

173. The pharmaceutical composition of claim 162, wherein the composition comprises an activator of E2F3, an activator of EZH2 and an inhibitor of ZFX.

174. The pharmaceutical composition of claim 162, wherein the composition comprises an activator of EZH2, an inhibitor of STAT3, and an inhibitor of ZFX.

175. The pharmaceutical composition of claim 162, wherein the composition comprises an activator of E2F3, an activator of EZH2, an inhibitor of STAT3 and an inhibitor ZFX.

176. The pharmaceutical composition of claim 162, wherein the activator comprises a polynucleic acid encoding the transcription factor or an expression plasmid comprising the polynucleic acid encoding the transcription factor.

177. The pharmaceutical composition of claim 162, wherein the inhibitor comprises a CRISPR system, a repression plasmid, an antisense oligonucleotide, or siRNA targeting the transcription factor; or a polynucleic acid encoding the CRISPR system, the antisense oligonucleotide, or the siRNA.

178. A lipid nanoparticle comprising the composition of claim 128-143.

179. A cell comprising the composition of any one of claim 128-143.

180. A cell produced by the method of any one of claims 1-127.

181. A polynucleotide comprising the composition of any one of claims 128-178.

182. A pharmaceutical composition comprising (i) the composition of any one of claims 128-143, the primary cell of any one of claims 144-161, the cell of claim 179 or claim 180, or the polynucleotide of claim 181, and (ii) a pharmaceutically acceptable carrier, excipient, or diluent.

183. A method of treating a disorder in a subject in need thereof, the method comprising administration of the pharmaceutical composition of any one of claims 162-177 or the pharmaceutical composition of claim 182 to the subject in need thereof.

184. The method of claim 183, wherein the disorder is an age-related disorder.

185. A kit, comprising:
(a) at least one activator of a transcription factor, wherein the transcription factor is selected from the group consisting of:
(i) E2F Transcription Factor 3 (E2F3);
(ii) Enhancer Of Zeste 2 Polycomb Repressive Complex 2 Subunit (EZH2);
(iii) Homeobox protein DLX-6 (DLX6);
(iv) Forkhead Box M1 (FOXM1);
(v) FOS Like 1, AP-1 Transcription Factor Subunit (FOSL1);
(vi) Nuclear Factor Of Activated T Cells 4 (NFATC4);
(vii) Myc proto-oncogene protein (MYC);
(viii) Signal Transducer And Activator Of Transcription 4 (STAT4);
(ix) GATA Binding Protein 3 (GATA3);
(x) Heat Shock Transcription Factor 2 (HSF2);
(xi) Paired Box 4 (PAX4);
(xii) NK2 Homeobox 2 (NKX2-2);
(xiii) SIM BHLH Transcription Factor 2 (SIM2); and
(xiv) Ventral Anterior Homeobox 1 (VAX1);
and
(b) at least one inhibitor of a transcription factor, wherein the transcription factor is selected from the group consisting of:
(i) Signal Transducer And Activator Of Transcription 3 (STAT3);
(ii) Zinc Finger Protein X-Linked (ZFX);
(iii) Early growth response protein 1 (EGR1);
(iv) Activating Transcription Factor 2 (ATF2);
(v) Vascular Endothelial Zinc Finger 1 (VEZF1);
(vi) MYC Associated Zinc Finger Protein (MAZ);
(vii) SRY-Box Transcription Factor 2 (SOX2);
(viii) Paired Box 8 (PAX8);
(ix) Zinc Finger Homeobox 3 (ZFHX3);
(x) Autophagy Related 4C Cysteine Peptidase (ATG4C);
(xi) Autophagy Related 5 (ATG5);
(xii) High Mobility Group Box 1 (HMGB1);
(xiii) GATA Binding Protein 2 (GATA2); and
(xiv) KLF Transcription Factor 4 (KLF4).

186. The kit of claim 185, wherein the kit comprises:
(a) at least one activator of a transcription factor of EZH2, E2F3, FOXM1, or DLX6; and
(b) at least one inhibitor of a transcription factor of STAT3, ZFX, EGR1, MAZ, SOX2, or ATF4.

187. The kit of claim 185, wherein the kit does not comprise an activator of a transcription factor of OCT4, SOX2, KLF4, or MYC.

188. The kit of claim 185, wherein the kit comprises (a) one or both activator of a transcription factor of EZH2 or E2F3; and (b) one or both inhibitor of a transcription factor of STAT3 or ZFX.

189. The kit of claim 185, wherein the kit comprises an activator of E2F3 and an activator of EZH2.

190. The kit of claim 185, wherein the kit comprises an inhibitor of STAT3 and an inhibitor of ZFX.

191. The kit of claim 185, wherein the kit comprises an activator of E2F3 and an inhibitor of STAT3.

192. The kit of claim 185, wherein the kit comprises an activator of E2F3 and an inhibitor of ZFX.

193. The kit of claim 185, wherein the kit comprises an activator of EZH2 and an inhibitor of STAT3.

194. The kit of claim 185, wherein the kit comprises an activator of EZH2 and an inhibitor of ZFX.

195. The kit of claim 185, wherein the kit comprises an activator of E2F3, an activator of EZH2 and an inhibitor of STAT3.

196. The kit of claim 185, wherein the kit comprises an activator of E2F3, an activator of EZH2 and an inhibitor of ZFX.

197. The kit of claim 185, wherein the kit comprises an activator of EZH2, an inhibitor of STAT3, and an inhibitor of ZFX.

198. The kit of claim 185, wherein the kit comprises an activator of E2F3, an activator of EZH2, an inhibitor of STAT3 and an inhibitor ZFX.

199. The kit of claim 185, wherein the activator comprises a polynucleic acid encoding the transcription factor or an expression plasmid comprising the polynucleic acid encoding the transcription factor.

200. The kit of claim 185, wherein the inhibitor comprises a CRISPR system, a repression plasmid, an antisense oligonucleotide, or siRNA; or a polynucleic acid encoding the CRISPR system, the antisense oligonucleotide, or the siRNA.

201. A method for rejuvenating a cell, the method comprising increasing the activity of one or more transcription factors in the cell, wherein the one or more transcription factors are selected from the group consisting of:
(i) E2F Transcription Factor 3 (E2F3);
(ii) Enhancer Of Zeste 2 Polycomb Repressive Complex 2 Subunit (EZH2);
(iii) Homeobox protein DLX-6 (DLX6);
(iv) Forkhead Box M1 (FOXM1);
(v) FOS Like 1, AP-1 Transcription Factor Subunit (FOSL1);
(vi) Nuclear Factor Of Activated T Cells 4 (NFATC4);
(vii) Myc proto-oncogene protein (MYC);
(viii) Signal Transducer And Activator Of Transcription 4 (STAT4);
(ix) GATA Binding Protein 3 (GATA3);
(x) Heat Shock Transcription Factor 2 (HSF2);
(xi) Paired Box 4 (PAX4);
(xii) NK2 Homeobox 2 (NKX2-2);
(xiii) SIM BHLH Transcription Factor 2 (SIM2); and
(xiv) Ventral Anterior Homeobox 1 (VAX1);
and/or wherein the method comprising decreasing the activity of one or more transcription factors in the cell, wherein the transcription factors are selected from the group consisting of:
(i) Signal Transducer And Activator Of Transcription 3 (STAT3);
(ii) Zinc Finger Protein X-Linked (ZFX);
(iii) Early growth response protein 1 (EGR1);
(iv) Activating Transcription Factor 2 (ATF2);
(v) Vascular Endothelial Zinc Finger 1 (VEZF1);
(vi) MYC Associated Zinc Finger Protein (MAZ);
(vii) SRY-Box Transcription Factor 2 (SOX2);
(viii) Paired Box 8 (PAX8);
(ix) Zinc Finger Homeobox 3 (ZFHX3);
(x) Autophagy Related 4C Cysteine Peptidase (ATG4C);
(xi) Autophagy Related 5 (ATG5);
(xii) High Mobility Group Box 1 (HMGB1);
(xiii) GATA Binding Protein 2 (GATA2); and
(xiv) KLF Transcription Factor 4 (KLF4).

202. The method of claim 201, wherein the one or more transcription factors are selected from the group consisting of: STAT3, ZFX, EGR1, MAZ, SOX2, and ATF4.

203. The method of claim 201, wherein the method comprising increasing the activity of one or more transcription factors in the cell, wherein the one or more transcription factors are selected from the group consisting of E2F3, EZH2, FOXM1, and DLX6; and
wherein the method comprising decreasing activity of one or more transcription factors in the cell, wherein the transcription factors are selected from the group consisting of STAT3, ZFX, EGR1, MAZ, SOX2, and ATF4.

204. The method of claim 203, wherein the method comprising increasing the activity of one or more transcription factors in the cell, wherein the one or more transcription factors are E2F3 or EZH2; and
wherein the method comprising decreasing the activity of one or more transcription factors in the cell, wherein the transcription factors are STAT3 or ZFX.

205. The method of claim 201, wherein the method comprises increasing the activities of E2F3 and EZH2.

206. The method of claim 201, wherein the method comprises decreasing the activities of STAT3 and ZFX.

207. The method of claim 201, wherein the method comprises increasing the activity of E2F3 and decreasing the activity of STAT3.

208. The method of claim 201, wherein the method comprises increasing the activity of E2F3 and decreasing the activity of ZFX.

209. The method of claim 201, wherein the method comprises increasing the activity of EZH2 and decreasing the activity of STAT3.

210. The method of claim 201, wherein the method comprises increasing the activities of EZH2 and decreasing the activity of ZFX.

211. The method of claim 201, wherein the method comprises increasing the activities of E2F3 and EZH2 and decreasing the activity of STAT3.

212. The method of claim 201, wherein the method comprises increasing the activities of E2F3 and EZH2 and decreasing the activity of ZFX.

213. The method of claim 201, wherein the method comprises increasing the activity of EZH2 and decreasing the activities of STAT3 and ZFX.

214. The method of claim 201, wherein the method comprises increasing the activities of E2F3 and EZH2 and decreasing the activities of STAT3 and ZFX.

215. The method of claim 201, wherein the activity of any two of the transcription factors is increased.

216. The method of claim 201, wherein the activity of at least two of the transcription factors is increased.

217. The method of claim 201, wherein the activity of any two of the transcription factors is decreased.

218. The method of claim 201, wherein the activity of at least two of the transcription factors is decreased.

219. The method of claim 201, wherein the activity of any two of the transcription factors is increased or decreased.

220. The method of claim 201, wherein the activity of at least two of the transcription factors is increased.
